(19) United States
(12) Patent Application Publication
(10) Pub. No.: US 2025/0059300 A1
(43) Pub. Date: Feb. 20, 2025

(54) METHODS FOR TREATING PATIENTS WITH HETEROZYGOUS FAMILIAL HYPERCHOLESTEROLEMIA (heFH) WITH AN ANTI-PCSK9 ANTIBODY

(71) Applicants: Sanofi Biotechnology, Paris (FR); Regeneron Pharmaceuticals, Inc., Tarrytown, NY (US)

(72) Inventors: Marie Baccara-Dinet, Paris (FR); Corinne Hanotin, Paris (FR); Laurence Bessac, Paris (FR); Umesh Chaudhari, Bridgewater, NJ (US); Robert C. Pordy, Tarrytown, NY (US); William J. Sasiela, Tarrytown, NY (US); Daniel A. Schwemmer Gipe, Tarrytown, NY (US)

(21) Appl. No.: 18/786,917

(22) Filed: Jul. 29, 2024

Related U.S. Application Data
(63) Continuation of application No. 17/693,837, filed on Mar. 14, 2022, now abandoned.
Application No. 17/693,837 is a continuation of application No. 16/707,492, filed on Dec. 9, 2019, now Pat. No. 11,306,155.
Application No. 16/707,492 is a continuation of application No. 14/801,384, filed on Jul. 16, 2015, now Pat. No. 10,544,232.
(60) Provisional application No. 62/080,717, filed on Nov. 17, 2014.
(60) Provisional application No. 62/043,144, filed on Aug. 28, 2014.
(60) Provisional application No. 62/025,362, filed on Jul. 16, 2014.

(30) Foreign Application Priority Data
Mar. 23, 2015 (EP) 15305419.2

(51) Int. Cl. C07K 16/40; A61K 39/00; A61K 39/395; A61P 3/06; C07K 14/705

(52) U.S. Cl. CPC C07K 16/40; A61K 39/3955; A61P 3/06; A61K 2039/505; A61K 2039/545; C07K 14/705; C07K 2317/21; C07K 2317/565; C07K 2317/76

(57) ABSTRACT

The present invention provides methods for treating hypercholesterolemia. The methods of the present invention comprise administering to patients with heterozygous familial hypercholesterolemia a pharmaceutical composition comprising a PCSK9 inhibitor. In certain embodiments, the PCSK9 inhibitor is an anti-PCSK9 antibody such as the exemplary antibody referred to herein as mAb316P. The methods of the present invention are useful for treating patients with heterozygous familial hypercholesterolemia who are not adequately controlled by maximum tolerated dose statin therapy with or without other lipid lowering therapy.

### RELATED APPLICATIONS

This application is a continuation of U.S. patent application Ser. No. 17/693,837, filed Mar. 14, 2022, which is a continuation of U.S. patent application Ser. No. 16/707,492, filed Dec. 9, 2019, now U.S. Pat. No. 11,306,155, which is a continuation of U.S. patent application Ser. No. 14/801,384, filed Jul. 16, 2015, now U.S. Pat. No. 10,544,232, which claims the benefit of U.S. Provisional Application No. 62/080,717, filed on Nov. 17, 2014, U.S. Provisional Application No. 62/043,144, filed on Aug. 28, 2014, U.S. Provisional Application No. 62/025,362, filed on Jul. 16, 2014, and European Patent Application No. 15305419.2, filed on Mar. 23, 2015, the contents of which are incorporated herein by reference in their entirety.

### SEQUENCE LISTING

The instant application contains a Sequence Listing which has been submitted electronically in XML format and is hereby incorporated by reference in its entirety. Said XML file, created on Jul. 29, 2024, is named 756541_SA9-147CON3_ST26.xml and is 229,283 bytes in size.

### FIELD OF THE INVENTION

The present invention relates to the field of therapeutic treatments of diseases and disorders that are associated with elevated levels of lipids and lipoproteins. More specifically, the invention relates to the use of PCSK9 inhibitors to treat patients with heterozygous familial hypercholesterolemia who are not adequately controlled by maximum tolerated dose statin therapy with or without other lipid lowering therapy.

### BACKGROUND

Heterozygous familial hypercholesterolemia (heFH) is a hereditary lipid metabolism disorder that predisposes affected individuals to cardiovascular (CV) disease. Patients with heFH typically have very high low-density lipoprotein cholesterol (LDL-C) levels—often >190 mg/dL at the time of diagnosis—that are associated with high risk for premature CV disease. Findings from observational studies have shown that the risk of coronary heart disease (CHD) is reduced in heFH patients receiving statin therapy; however, even with treatment, the risk of CHD is still greater in heFH patients than in the general population. Despite the availability of lipid-lowering therapy (LLT), approximately 80% of patients with heFH do not reach the recommended levels of LDL-C. Given the increased CV risk in the heFH population, there is a need to provide patients with more intensive cholesterol-lowering therapy.

Current LDL-C lowering medications include statins, cholesterol absorption inhibitors (e.g., ezetimibe [EZE]), fibrates, niacin, and bile acid sequestrants. Statins are the most commonly prescribed, as they have shown a greater ability to lower LDL-C and reduce CHD events. However, many patients at risk of cardiovascular disease (CVD) have poorly controlled low-density lipoprotein cholesterol (LDL-C) despite statin therapy.

### BRIEF SUMMARY OF THE INVENTION

The present invention provides methods for treating hypercholesterolemia. In particular, the methods of the present invention are useful for treating patients with heterozygous familial hypercholesterolemia who are not adequately controlled by maximum tolerated dose statin therapy with or without other lipid lowering therapy.

According to one aspect, the methods of the present invention comprise administering one or more doses of a PCSK9 inhibitor to a patient with heterozygous familial hypercholesterolemia who is not adequately controlled by maximum tolerated dose statin therapy with or without other lipid lowering therapy (i.e., hypercholesterolemia that is not adequately controlled by maximum tolerated dose statin therapy in the absence of a PCSK9 inhibitor, with or without other lipid modifying therapy). According to certain embodiments of the present invention, the PCSK9 inhibitor is administered to the patient with heterozygous familial hypercholesterolemia as an add-on therapy to the patient's existing statin therapy with or without other lipid lowering therapy.

According to another aspect, the methods of the present invention comprise selecting a patient with heterozygous familial hypercholesterolemia who is not adequately controlled by maximum tolerated dose statin therapy with or without other lipid lowering therapy (e.g., a maximum tolerated dose statin therapy), and administering to the patient one or more doses of a PCSK9 inhibitor in combination with (i.e., “on top of”) the statin therapy.

Another aspect of the invention includes a method for treating a patient with heterozygous familial hypercholesterolemia (heFH) who is not adequately controlled by maximum tolerated dose statin therapy with or without other lipid lowering therapy by administering one or more doses of a proprotein convertase subtilisin/kexin type 9 (PCSK9) inhibitor to the patient, wherein the patient exhibits inadequate control of the hypercholesterolemia despite treatment with the maximum tolerated dose statin therapy with or without other lipid lowering therapy in the absence of the PCSK9 inhibitor.

Another aspect of the invention includes a method for reducing low-density lipoprotein cholesterol (LDL-C) in a patient with heterozygous familial hypercholesterolemia (heFH) who is not adequately controlled by maximum tolerated dose statin therapy with or without other lipid lowering therapy by administering one or more doses of a proprotein convertase subtilisin/kexin type 9 (PCSK9) inhibitor to the patient, wherein the patient exhibits inadequate control of the hypercholesterolemia despite treatment with the maximum tolerated dose statin therapy with or without other lipid lowering therapy in the absence of the PCSK9 inhibitor.

Another aspect of the invention includes a method for treating hypercholesterolemia in a patient with heterozygous familial hypercholesterolemia (heFH) who is not adequately controlled by maximum tolerated dose statin therapy with or without other lipid lowering therapy by administering one or more doses of a proprotein convertase subtilisin/kexin type 9 (PCSK9) inhibitor to the patient, wherein the patient exhibits inadequate control of the hypercholesterolemia despite treatment with the maximum tolerated dose statin therapy with or without other lipid lowering therapy in the absence of the PCSK9 inhibitor.

Another aspect of the invention includes a method for improving the serum level of one or more lipid components in a patient with heterozygous familial hypercholesterolemia (heFH) who is not adequately controlled by maximum tolerated dose statin therapy with or without other lipid lowering therapy by administering one or more doses of a proprotein convertase subtilisin/kexin type 9 (PCSK9) inhibitor to the patient, wherein the patient exhibits inadequate control of the lipid component despite treatment with the maximum tolerated dose statin therapy with or without other lipid lowering therapy in the absence of the PCSK9 inhibitor. In certain aspects, the invention provides a decrease in the serum level of a lipid component selected from the group consisting of LDL-C, Apo B, non-HDL-C, total cholesterol, Lp(a), and triglycerides. In certain aspects, the invention provides an increase in the serum level of a lipid component selected from the group consisting of HDL-C and Apo A1.

In certain aspects of the invention, the diagnosis of heFH is made by either genotyping or by clinical criteria. In some aspects, the clinical criteria is either the Simon Broome Register Diagnostic Criteria for Heterozygous Familial Hypercholesterolemia, or the WHO/Dutch Lipid Network criteria with a score >8.

In certain aspects of the invention, the PCSK9 inhibitor is an antibody or an antigen-binding fragment thereof that specifically binds PCSK9.

In certain aspects of the invention, the antibody or antigen-binding fragment thereof comprises the heavy and light chain complementarity determining regions (CDRs) of a heavy chain variable region/light chain variable region (HCVR/LCVR) amino acid sequence pair selected from the group consisting of SEQ ID NOs: 1/6 and 11/15. In some aspects, the antibody or antigen-binding fragment thereof comprises heavy and light chain CDR amino acid sequences having SEQ ID NOs:12, 13, 14, 16, 17, and 18. In some aspects, the antibody or antigen-binding fragment thereof comprises an HCVR having the amino acid sequence of SEQ ID NO:11 and an LCVR having the amino acid sequence of SEQ ID NO:15. In some aspects, the antibody or antigen-binding fragment thereof comprises heavy and light chain CDR amino acid sequences having SEQ ID NOs:2, 3, 4, 7, 8, and 10. In some aspects, the antibody or antigen-binding fragment thereof comprises an HCVR having the amino acid sequence of SEQ ID NO:1 and an LCVR having the amino acid sequence of SEQ ID NO:6.

In certain aspects of the invention, the antibody or antigen-binding fragment thereof binds to the same epitope on PCSK9 as an antibody comprising heavy and light chain CDR amino acid sequences having SEQ ID NOs:12, 13, 14, 16, 17, and 18; or SEQ ID NOs: 2, 3, 4, 7, 8, and 10.

In certain aspects of the invention, the antibody or antigen-binding fragment thereof competes for binding to PCSK9 with an antibody comprising heavy and light chain CDR amino acid sequences having SEQ ID NOs:12, 13, 14, 16, 17, and 18; or SEQ ID NOs: 2, 3, 4, 7, 8, and 10.

In certain aspects of the invention, the antibody or antigen-binding fragment thereof that specifically binds PCSK9 is administered to the patient at a dose of about 75 mg at a frequency of once every two weeks. In some aspects, the about 75 mg dose is maintained if the patient's LDL-C measured after five or more doses is <70 mg/dL. In some aspects, the about 75 mg dose is discontinued if the patient's LDL-C measured after five or more doses remains ≥70 mg/dL, and the antibody or antigen-binding fragment thereof that specifically binds PCSK9 is subsequently administered to the patient at a dose of about 150 mg at a frequency of once every two weeks. In some aspects, the antibody or antigen-binding fragment thereof that specifically binds PCSK9 is administered to the patient at a dose of about 150 mg at a frequency of once every two weeks.

In certain aspects of the invention, the PCSK9 inhibitor is administered to the patient in combination with the maximum tolerated dose statin therapy. In some aspects, the maximum tolerated dose statin therapy comprises a daily dose of about 40 mg to about 80 mg of atorvastatin. In some aspects, the maximum tolerated dose statin therapy comprises a daily dose of about 20 mg to about 40 mg of rosuvastatin. In some aspects, the maximum tolerated dose statin therapy comprises a daily dose of about 80 mg of simvastatin.

In certain aspects of the invention, the PCSK9 inhibitor is administered to the patient in combination with the other lipid lowering therapy.

In certain aspects of the invention, the method improves at least one hypercholesterolemia-associated parameter selected from the group consisting of: (a) reduction of the patient's low density lipoprotein cholesterol (LDL-C) by at least 40%; (b) reduction of the patient's apolipoprotein B (ApoB) by at least 30%; (c) reduction of the patient's non-high density lipoproprotein cholesterol (non-HDL-C) by at least 40%; (d) reduction of the patient's total cholesterol by at least 20%; (e) increase of the patient's high density lipoprotein cholesterol (HDL-C) by at least 3%; (f) reduction of the patient's triglycerides by at least 5%; (g) reduction of the patient's lipoprotein a (Lp(a)) by at least 20%; and (h) increase of the patient's apolipoprotein A1 by at least 1%.

Other embodiments of the present invention will become apparent from a review of the ensuing detailed description.

### BRIEF DESCRIPTION OF THE FIGURES

FIG. 1 is a graphic representation of the study design for ODYSSEY FH I (Example 2).

FIG. 2 is a graph showing the calculated LDL-C LS mean percent change from baseline over time for treatment with alirocumab or placebo in the ITT population in the ODYSSEY FH I study (Example 2). The least-squares (LS) means and standard errors (SD) are taken from MMRM (mixed-effect model with repeated measures) analysis.

FIG. 3 is a graphic representation of the study design for ODYSSEY FH II (Example 3).

FIG. 4 is a graph showing the LDL-C LS mean (+/−SE) percent change from baseline over time for the ITT population in the ODYSSEY FH II study (Example 3). The Least-squares (LS) means and standard errors (SE) taken from MMRM (mixed-effect model with repeated measures) analysis.

FIG. 5 is a graph showing the LDL-C LS mean (+/−SE) percent change from baseline during efficacy treatment period over time for the mITT Population in the ODYSSEY FH II study (Example 3).

FIG. 6 is a graphic representation of the study design for ODYSSEY HIGH FH (Example 4). Labels in the study design are defined as follows: FU: follow up; HeFH, heterozygous familial hypercholesterolemia; LLT, lipid-lowering therapy; OLE, open-label extension.

FIG. 7 is a graph showing the calculated LDL-C LS mean percent change from baseline over time for treatment with alirocumab or placebo in the ITT population in the ODYSSEY HIGH FH study (Example 4). The least-squares (LS) means and standard errors (SE) are taken from MMRM (mixed-effect model with repeated measures) analysis.

FIG. 8 is a graph showing the LS mean (SE) calculated LDL-C values versus time for the ODYSSEY FH I and FH II studies. The values indicted on the graph are the LS mean % change from baseline to week 24 and week 52.

FIG. 9 is a graph showing the LS mean (SE) calculated LDL-C values versus time for the ODYSSEY FH I and FH II studies. The values indicated below the graph are the numbers of patients analyzed at the various timepoints.

FIG. 10 is a graph showing LDL-C levels over time in alirocumab patients according to whether dose was increased to 150 mg Q2W or maintained at 75 mg Q2W (ITT analysis).

FIGS. 11A, 11B, and 11C depict charts showing subgroup analysis of LDL-C reductions from baseline to week 24 (alirocumab vs. placebo) according to demographics and baseline characteristics (FIG. 11A), statin/LLT use (FIG. 11B), and baseline lipids (FIG. 11C) (ITT analysis; pooled data from FH I and FH II). Moderate chronic kidney disease (CKD) was defined as an estimated glomerular filtration rate of ≥30 and 560 mL/min/1.73 m2. In FH I, 20/323 and 9/163 patients in alirocumab and placebo arms had moderate CKD at baseline. Corresponding values in FH II were 2/167 and 1/82. “High intensity” statin dose refers to atorvastatin 40-80 mg or rosuvastatin 20-40 mg.

FIG. 12 is a graphic representation of patient disposition in the ODYSSEY HIGH FH study.

FIG. 13 is a graph showing the percent change from baseline to week 24 in LDL-C levels by individual patients in the ODYSSEY HIGH FH study. All patients were on a background statin (at the maximum tolerated level). A subset of patients also received a further lipid lowering therapy.

FIGS. 14A-14B depict graphs showing the LS mean (SE) calculated LDL-C values versus time for the ODYSSEY HIGH FH study. In FIG. 14A, the values indicted on the graph are the LS mean % values (in mg/dL) at week 24 and week 52. In FIG. 14B, the values indicated on the graph are the LS mean % values (in mg/dL) at week 24 and week 78. All patients were on a background statin (at the maximum tolerated level). A subset of patients also received a further lipid lowering therapy.

### DETAILED DESCRIPTION

Before the present invention is described, it is to be understood that this invention is not limited to the particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

Heterozygous Familial Hypercholesterolemia not Adequately Controlled by Maximum Tolerated Dose Statin Therapy with or without Other Lipid Lowering Therapy

The present invention relates generally to methods and compositions for treating patients with heterozygous familial hypercholesterolemia who are not adequately controlled by maximum tolerated dose statin therapy with or without other lipid lowering therapy, i.e., hypercholesterolemia not adequately controlled by a therapeutic regimen comprising a daily maximum tolerated dose of a statin. As used herein, the expression “not adequately controlled,” in reference to hypercholesterolemia, means that the patient's serum low-density lipoprotein cholesterol (LDL-C) concentration, total cholesterol concentration, and/or triglyceride concentration is not reduced to a recognized, medically-acceptable level (taking into account the patient's relative risk of coronary heart disease) after at least 4 weeks on a therapeutic regimen comprising a stable daily dose of a statin. For example, “a patient with hypercholesterolemia that is not adequately controlled by a statin” includes patients with a serum LDL-C concentration of greater than about 70 mg/dL, 100 mg/dL, 130 mg/dL, 140 mg/dL, or more (depending on the patient's underlying risk of heart disease) after the patient has been on a stable daily statin regimen for at least 4 weeks.

According to certain embodiments, the patients with heterozygous familial hypercholesterolemia who are not adequately controlled by maximum tolerated dose statin therapy with or without other lipid lowering therapy who are treatable by the methods of the present invention have hypercholesterolemia (e.g., a serum LDL-C concentration of greater than or equal to 70 mg/dL in patients with a history of documented cardiovascular disease or a serum LDL-C≥100 mg/dL in patients without a history of documented cardiovascular disease) despite taking a stable daily dose of a statin (with or without other lipid modifying therapy) for at least 4 weeks, 5 weeks, 6 weeks, or more. In certain embodiments, the heterozygous familial hypercholesterolemia patient's hypercholesterolemia is inadequately controlled by a maximum tolerated dose statin therapy (also referred to herein as “a daily maximum tolerated dose therapeutic statin regimen”).

As used herein, “maximum tolerated dose statin therapy” means a therapeutic regimen comprising the administration of daily dose of a statin that is the maximally tolerated dose for a particular patient. Maximally tolerated dose means the highest dose of statin that can be administered to a patient without causing unacceptable adverse side effects in the patient. Maximum tolerated dose statin therapy includes, but is not limited to, e.g., 40-80 mg of atorvastatin daily, 20-40 mg of rosuvastatin daily, or 80 mg of simvastatin (if already on this dose for >1 year). However, patients not able to tolerate the above statin doses could take a lower dose of daily atorvastatin, rosuvastatin, or simvastatin provided there was an acceptable reason for not using the higher dose. Some examples of acceptable reasons for a patient taking a lower statin dose include: adverse effects on higher doses, advanced age, low body mass index (BMI), regional practices, local prescribing information, concomitant medications, and comorbid conditions such as impaired glucose tolerance/impaired fasting glucose.

The present invention also includes methods for treating patients with heterozygous familial hypercholesterolemia that are not adequately controlled by maximum tolerated dose statin therapy with or without other lipid lowering therapy comprising daily administration of other statins such as cerivastatin, pitavastatin, fluvastatin, lovastatin, and pravastatin.

### Patient Selection

The present invention includes methods and compositions useful for treating patients with heterozygous familial hypercholesterolemia who are not adequately controlled by maximum tolerated dose statin therapy with or without other lipid lowering therapy.

Diagnosis of heFH must be made either by genotyping or by clinical criteria. For those patients not genotyped, the clinical diagnosis may be based on either the Simon Broome criteria with a criteria for definite FH or the WHO/Dutch Lipid Network criteria with a score >8 points.

According to the Simon Broome Register Diagnostic Criteria for Heterozygous Familial Hypercholesterolemia, definite familial hypercholesterolemia is defined as: 1) total-C>6.7 mmol/l (260 mg/dL) or LDL cholesterol above 4.0 mmol/l (155 mg/dL) in a child <16 years or Total-C>7.5 mmol/l (290 mg/dL) or LDL cholesterol above 4.9 mmol/l (190 mg/dL) in an adult. (Levels either pre-treatment or highest on treatment); plus either A) tendon xanthomas in patient, or in 1st degree relative (parent, sibling, child), or in 2nd degree relative (grandparent, uncle, aunt); or B) DNA-based evidence of an LDL receptor mutation or familial defective apo B-100.

According to the Simon Broome Register Diagnostic Criteria for Heterozygous Familial Hypercholesterolemia, possible familial hypercholesterolemia is defined as: 1) total-C>6.7 mmol/l (260 mg/dL) or LDL cholesterol above 4.0 mmol/l (155 mg/dL) in a child <16 years or Total-C>7.5 mmol/l (290 mg/dL) or LDL cholesterol above 4.9 mmol/l (190 mg/dL) in an adult. (Levels either pre-treatment or highest on treatment); and at least one of the following: A) family history of MI below 50 years of age in 2nd degree relative or below 60 years of age in 1st degree relative; and B) family history of raised cholesterols >7.5 mmol/l (290 mg/dL) in adult 1st or 2nd degree relative or >6.7 mmol/l (260 mg/dL) in child or sibling under 16 years of age.

The WHO Criteria (Dutch Lipid Network clinical criteria) for diagnosis of Heterozygous Familial Hypercholesterolemia (heFH) is set forth in the Examples, such as in Table 2.

According to certain embodiments, the heterozygous familial hypercholesterolemia patient may be selected on the basis of having one or more additional risk factors selected from the group consisting of age (e.g., older than 40, 45, 50, 55, 60, 65, 70, 75, or 80 years), race, national origin, gender (male or female), exercise habits (e.g., regular exerciser, non-exerciser), other preexisting medical conditions (e.g., type-II diabetes, high blood pressure, myocardial infarction, ischemic stroke, etc.), and current medication status (e.g., currently taking beta blockers, niacin, ezetimibe, fibrates, omega-3 fatty acids, bile acid resins, etc.).

According to the present invention, heterozygous familial hypercholesterolemia patients may be selected on the basis of a combination of one or more of the foregoing selection criteria or therapeutic characteristics.

### Administration of a PCSK9 Inhibitor as Add-on Therapy to Maximum Tolerated Dose Statin Therapy

The present invention includes methods wherein a heterozygous familial hypercholesterolemia patient who is not adequately controlled by maximum tolerated dose statin therapy with or without other lipid lowering therapy in the absence of a PCSK9 inhibitor is administered a PCSK9 inhibitor according to a particular dosing amount and frequency, and wherein the PCSK9 inhibitor is administered as an add-on to the patient's therapeutic statin regimen. For example, according to certain embodiments, if a patient with heterozygous familial hypercholesterolemia who is not adequately controlled by maximum tolerated dose statin therapy with or without other lipid lowering therapy comprising, e.g., 40-80 mg of atorvastatin, the patient with heterozygous familial hypercholesterolemia may be administered a PCSK9 inhibitor at a particular amount and dosing interval while the patient continues his or her stable daily therapeutic statin regimen.

The methods of the present invention include add-on therapeutic regimens wherein the PCSK9 inhibitor is administered as add-on therapy to the same stable daily maximum tolerated dose therapeutic statin regimen (i.e., same dosing amount of statin) that the heterozygous familial hypercholesterolemia risk patient was on prior to receiving the PCSK9 inhibitor. In other embodiments, the PCSK9 inhibitor is administered as add-on therapy to a daily maximum tolerated dose therapeutic statin regimen comprising a statin in an amount that is more than or less than the dose of statin the patient was on prior to receiving the PCSK9 inhibitor. For example, after starting a therapeutic regimen comprising a PCSK9 inhibitor administered at a particular dosing frequency and amount, the daily dose of statin administered or prescribed to the patient may (a) stay the same, (b) increase, or (c) decrease (e.g., up-titrate or down-titrate) in comparison to the daily statin dose the high cardiovascular risk patient was taking before starting the PCSK9 inhibitor therapeutic regimen, depending on the therapeutic needs of the patient.

### Therapeutic Efficacy

The methods of the present invention will result in the improvement in the serum level of one or more lipid components selected from the group consisting of LDL-C, ApoB, non-HDL-C, total cholesterol, HDL-C, triglycerides, Apo A-1, and Lp(a). For example, according to certain embodiments of the present invention, administration of a pharmaceutical composition comprising a PCSK9 inhibitor to a heterozygous familial hypercholesterolemia patient who is not adequately controlled by a stable daily maximum tolerated dose therapeutic statin regimen (e.g., administration of the PCSK9 inhibitor on top of the patient's maximum tolerated dose statin therapy) will result in a mean percent reduction from baseline in serum low density lipoprotein cholesterol (LDL-C) of at least about 40%, 41%, 42%, 43%, 44%, 45%, 46%, 47%, 48%, 49%, 50%, 51%, 52%, 53%, 54%, 55%, or greater; a mean percent reduction from baseline in ApoB of at least about 30%, 31%, 32%, 33%, 34%, 35%, 36%, 37%, 38%, 39%, 40%, 41%, 42%, 43%, 44%, 45%, or greater; a mean percent reduction from baseline in non-HDL-C of at least about 40%, 41%, 42%, 43%, 44%, 45%, 46%, 47%, 48%, 49%, 50%, 51%, 52%, 53%, 54%, 55%, or greater; a mean percent reduction from baseline in total cholesterol of at least about 20%, 21%, 22%, 23%, 24%, 25%, 26%, 27%, 28%, 29%, 30%, 31%, 32%, 33%, 34%, 35%, or greater; a mean percent increase from baseline in HDL-C of at least about 1%, 2%, 3%, 4%, 5%, 6%, 7%, 8%, 9%, 10%, 11%, 12%, 13%, 14%, 15% or greater; a mean percent reduction from baseline in triglycerides of at least about 2%, 3%, 4%, 5%, 6%, 7%, 8%, 9%, 10%, 11%, 12%, 13%, 14%, 15%, or greater; a mean percent increase from baseline in Apo A-1 of at least about 1%, 2%, 3%, 4%, 5%, 6%, 7%, 8%, 9%, 10%, or greater; and/or a mean percent reduction from baseline in Lp(a) of at least about 20%, 21%, 22%, 23%, 24%, 25%, 26%, 27%, 28%, 29%, 30%, 31%, 32%, 33%, 34%, 35%, or greater.

### PCSK9 Inhibitors

The methods of the present invention comprise administering to a patient with heterozygous familial hypercholesterolemia who is not adequately controlled by maximum tolerated dose statin therapy with or without other lipid lowering therapy a therapeutic composition comprising a PCSK9 inhibitor. As used herein, a “PCSK9 inhibitor” is any agent that binds to or interacts with human PCSK9 and inhibits the normal biological function of PCSK9 in vitro or in vivo. Non-limiting examples of categories of PCSK9 inhibitors include small molecule PCSK9 antagonists, peptide-based PCSK9 antagonists (e.g., “peptibody” molecules), and antibodies or antigen-binding fragments of antibodies that specifically bind human PCSK9.

The term “human proprotein convertase subtilisin/kexin type 9” or “human PCSK9” or “hPCSK9”, as used herein, refers to PCSK9 having the nucleic acid sequence shown in SEQ ID NO:197 and the amino acid sequence of SEQ ID NO:198, or a biologically active fragment thereof.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-PCSK9 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PCSK9, as used in the context of the present invention, includes antibodies that bind PCSK9 or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PCSK9, however, have cross-reactivity to other antigens, such as PCSK9 molecules from other (non-human) species.

The anti-PCSK9 antibodies useful for the methods of the present invention may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-PCSK9 antibodies comprising variants of any of the HCVR, LCVR, and/or CDR amino acid sequences disclosed herein having one or more conservative substitutions. For example, the present invention includes the use of anti-PCSK9 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “KD”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

According to certain embodiments, the anti-PCSK9 antibody used in the methods of the present invention is an antibody with pH-dependent binding characteristics. As used herein, the expression “pH-dependent binding” means that the antibody or antigen-binding fragment thereof exhibits “reduced binding to PCSK9 at acidic pH as compared to neutral pH” (for purposes of the present disclosure, both expressions may be used interchangeably). For example, antibodies “with pH-dependent binding characteristics” includes antibodies and antigen-binding fragments thereof that bind PCSK9 with higher affinity at neutral pH than at acidic pH. In certain embodiments, the antibodies and antigen-binding fragments of the present invention bind PCSK9 with at least 3, 5, 10, 15, 20, 25, 30, 35, 40, 45, 50, 55, 60, 65, 70, 75, 80, 85, 90, 95, 100, or more times higher affinity at neutral pH than at acidic pH.

According to this aspect of the invention, the anti-PCSK9 antibodies with pH-dependent binding characteristics may possess one or more amino acid variations relative to the parental anti-PCSK9 antibody. For example, an anti-PCSK9 antibody with pH-dependent binding characteristics may contain one or more histidine substitutions or insertions, e.g., in one or more CDRs of a parental anti-PCSK9 antibody. Thus, according to certain embodiments of the present invention, methods are provided comprising administering an anti-PCSK9 antibody which comprises CDR amino acid sequences (e.g., heavy and light chain CDRs) which are identical to the CDR amino acid sequences of a parental anti-PCSK9 antibody, except for the substitution of one or more amino acids of one or more CDRs of the parental antibody with a histidine residue. The anti-PCSK9 antibodies with pH-dependent binding may possess, e.g., 1, 2, 3, 4, 5, 6, 7, 8, 9, or more histidine substitutions, either within a single CDR of a parental antibody or distributed throughout multiple (e.g., 2, 3, 4, 5, or 6) CDRs of a parental anti-PCSK9 antibody. For example, the present invention includes the use of anti-PCSK9 antibodies with pH-dependent binding comprising one or more histidine substitutions in HCDR1, one or more histidine substitutions in HCDR2, one or more histidine substitutions in HCDR3, one or more histidine substitutions in LCDR1, one or more histidine substitutions in LCDR2, and/or one or more histidine substitutions in LCDR3, of a parental anti-PCSK9 antibody.

As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.90, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

### Preparation of Human Antibodies

Methods for generating human antibodies in transgenic mice are known in the art. Any such known methods can be used in the context of the present invention to make human antibodies that specifically bind to human PCSK9.

Using VELOCIMMUNE® technology (see, for example, U.S. Pat. No. 6,596,541, Regeneron Pharmaceuticals) or any other known method for generating monoclonal antibodies, high affinity chimeric antibodies to PCSK9 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc, using standard procedures known to those skilled in the art. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies that can be used in the methods of the present invention possess high affinities, as described above, when measured by binding to antigen either immobilized on solid phase or in solution phase. The mouse constant regions are replaced with desired human constant regions to generate the fully human antibodies of the invention. While the constant region selected may vary according to specific use, high affinity antigen-binding and target specificity characteristics reside in the variable region.

Specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs: 1 and 11, or a substantially similar sequence thereof having at least 90%, at least 95%, at least 98% or at least 99% sequence identity. Alternatively, specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs 37, 45, 53, 61, 69, 77, 85, 93, 101, 109, 117, 125, 133, 141, 149, 157, 165, 173, 181, and 189, or a substantially similar sequence thereof having at least 90%, at least 95%, at least 98% or at least 99% sequence identity. The antibody or antigen-binding fragment may comprise the three light chain CDRs (LCVR1, LCVR2, LCVR3) contained within a light chain variable region (LCVR) having an amino acid sequence selected from the group consisting of SEQ ID NOs: 6 and 15, or a substantially similar sequence thereof having at least 90%, at least 95%, at least 98% or at least 99% sequence identity. Alternatively, the antibody or antigen-binding fragment may comprise the three light chain CDRs (LCVR1, LCVR2, LCVR3) contained within a light chain variable region (LCVR) having an amino acid sequence selected from the group consisting of SEQ ID NOs 41, 49, 57, 65, 73, 81, 89, 97, 105, 113, 121, 129, 137, 145, 153, 161, 169, 177, 185, and 193, or a substantially similar sequence thereof having at least 90%, at least 95%, at least 98% or at least 99% sequence identity.

In certain embodiments of the present invention, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs:1/6 and 11/15. Alternatively, in certain embodiments of the present invention, the antibody or antigen-binding protein comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs:37/41, 45/49, 53/57, 61/65, 69/73, 77/81, 85/89, 93/97, 101/105, 109/113, 117/121, 125/129, 133/137, 141/145, 149/153, 157/161, 165/169, 173/177, 181/185, and 189/193.

In certain embodiments of the present invention, the anti-PCSK9 antibody, or antigen-binding fragment thereof, that can be used in the methods of the present invention has HCDR1/HCDR2/HCDR3/LCDR1/LCDR2/LCDR3 amino acid sequences selected from SEQ ID NOs: 2/3/4/7/8/10 (mAb316P) and 12/13/14/16/17/18 (mAb300N) (See US Patent App. Publ No. 2010/0166768).

In certain embodiments of the present invention, the antibody or antigen-binding fragment thereof comprises HCVR/LCVR amino acid sequence pairs selected from the group consisting of SEQ ID NOs: 1/6 and 11/15. Alternatively, in certain embodiments of the present invention, the antibody or antigen-binding protein comprises HCVR/LCVR amino acid sequence pairs selected from the group consisting of SEQ ID NOs:37/41, 45/49, 53/57, 61/65, 69/73, 77/81, 85/89, 93/97, 101/105, 109/113, 117/121, 125/129, 133/137, 141/145, 149/153, 157/161, 165/169, 173/177, 181/185, and 189/193.

### Pharmaceutical Compositions and Methods of Administration

The present invention includes methods which comprise administering a PCSK9 inhibitor to a patient with heterozygous familial hypercholesterolemia who is not adequately controlled by maximum tolerated dose statin therapy with or without other lipid lowering therapy, wherein the PCSK9 inhibitor is contained within a pharmaceutical composition. The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of PCSK9 inhibitor (e.g., anti-PCSK9 antibody) administered to a patient with heterozygous familial hypercholesterolemia who is not adequately controlled by maximum tolerated dose statin therapy with or without other lipid lowering therapy according to the methods of the present invention is, generally, a therapeutically effective amount. As used herein, the phrase “therapeutically effective amount” means a dose of PCSK9 inhibitor that results in a detectable improvement (at least about 5%, 10%, 15%, 20%, 25%, 30%, 35%, 40%, 45%, 50%, 55%, 60%, 65%, 70%, 75%, or more from baseline) in one or more parameters selected from the group consisting of LDL-C, ApoB, non-HDL-C, total cholesterol, HLDL-C, triglycerides, Apo A-1, and Lp(a).

In the case of an anti-PCSK9 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PCSK9 antibody.

The amount of anti-PCSK9 antibody contained within the individual doses may be expressed in terms of milligrams of antibody per kilogram of patient body weight (i.e., mg/kg). For example, the anti-PCSK9 antibody may be administered to a patient at a dose of about 0.0001 to about 10 mg/kg of patient body weight.

### Combination Therapies

As described elsewhere herein, the methods of the present invention may comprise administering a PCSK9 inhibitor to patients with heterozygous familial hypercholesterolemia in combination with the patient's previously prescribed stable daily maximum tolerated dose therapeutic statin regimen. According to certain embodiments of the present invention, additional therapeutic agents, besides a statin, may be administered to the patient in combination with the PCSK9 inhibitor. Examples of such additional therapeutic agents include e.g., (1) an agent which inhibits cholesterol uptake and or bile acid re-absorption (e.g., ezetimibe); (2) an agent which increases lipoprotein catabolism (such as niacin); and/or (3) activators of the LXR transcription factor that plays a role in cholesterol elimination such as 22-hydroxycholesterol.

### Administration Regimens

According to certain embodiments of the present invention, multiple doses of a PCSK9 inhibitor (i.e., a pharmaceutical composition comprising a PCSK9 inhibitor) may be administered to a subject over a defined time course (e.g., on top of a daily therapeutic statin regimen). The methods according to this aspect of the invention comprise sequentially administering to a patient with heterozygous familial hypercholesterolemia who is not adequately controlled by maximum tolerated dose statin therapy with or without other lipid lowering therapy multiple doses of a PCSK9 inhibitor. As used herein, “sequentially administering” means that each dose of PCSK9 inhibitor is administered to the subject at a different point in time, e.g., on different days separated by a predetermined interval (e.g., hours, days, weeks or months). The present invention includes methods which comprise sequentially administering to the patient with heterozygous familial hypercholesterolemia a single initial dose of a PCSK9 inhibitor, followed by one or more secondary doses of the PCSK9 inhibitor, and optionally followed by one or more tertiary doses of the PCSK9 inhibitor.

The terms “initial dose,” “secondary doses,” and “tertiary doses,” refer to the temporal sequence of administration of the individual doses of a pharmaceutical composition comprising a PCSK9 inhibitor. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the PCSK9 inhibitor, but generally may differ from one another in terms of frequency of administration. In certain embodiments, however, the amount of PCSK9 inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

According to exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antigen-binding molecule which is administered to a patient prior to the administration of the very next dose in the sequence with no intervening doses.

The methods according to this aspect of the invention may comprise administering to a patient with heterozygous familial hypercholesterolemia any number of secondary and/or tertiary doses of a PCSK9 inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient with heterozygous familial hypercholesterolemia 1 to 2, 4, 6, 8 or more weeks after the immediately preceding dose. Similarly, in embodiments involving multiple tertiary doses, each tertiary dose may be administered at the same frequency as the other tertiary doses. For example, each tertiary dose may be administered to the patient 1 to 2, 4, 6, 8 or more weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The present invention includes administration regimens comprising an up-titration option (also referred to herein as “dose modification”). As used herein, an “up-titration option” means that, after receiving a particular number of doses of a PCSK9 inhibitor, if a patient has not achieved a specified reduction in one or more defined therapeutic parameters, the dose of the PCSK9 inhibitor is thereafter increased. For example, in the case of a therapeutic regimen comprising administration of 75 mg doses of an anti-PCSK9 antibody to a patient with heterozygous familial hypercholesterolemia who is not adequately controlled by maximum tolerated dose statin therapy with or without other lipid lowering therapy at a frequency of once every two weeks, if after 8 weeks (i.e., 5 doses administered at Week 0, Week 2 and Week 4, Week 6 and Week 8), the patient has not achieved a serum LDL-C concentration of less than 70 mg/dL, then the dose of anti-PCSK9 antibody is increased to e.g., 150 mg administered once every two weeks thereafter (e.g., starting at Week 12).

In certain embodiments, the anti-PCSK9 antibody is administered to a subject at a dose of about 75 mg every two weeks, for example for at least six doses.

In some embodiments, the antibody is administered to a subject at a dose of about 75 mg every two weeks for 12 weeks, and the dose remains at 75 mg every two weeks if, at week 8, the subject's LDL-C value was less than 70 mg/dl.

In other embodiments, the antibody is administered to a subject at a dose of about 75 mg every two weeks for 12 weeks, and the dose is titrated up to about 150 mg every two weeks if, at week 8, the subject's LDL-C value was greater than or equal to 70 mg/dl. In certain embodiments, the anti-PCSK9 antibody is administered to a subject at a dose of about 150 mg every two weeks, for example for at least six doses.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Human Antibodies to Human PCSK9

Human anti-PCSK9 antibodies were generated as described in U.S. Pat. No. 8,062,640. The exemplary PCSK9 inhibitor used in the following Examples is the human anti-PCSK9 antibody designated “mAb316P,” also known as “Alirocumab.” mAb316P has the following amino acid sequence characteristics: heavy chain variable region (HCVR) comprising SEQ ID NO:1; light chain variable domain (LCVR) comprising SEQ ID NO:6; heavy chain complementarity determining region 1 (HCDR1) comprising SEQ ID NO:2; HCDR2 comprising SEQ ID NO:3; HCDR3 comprising SEQ ID NO:4; light chain complementarity determining region 1 (LCDR1) comprising SEQ ID NO:7; LCDR2 comprising SEQ ID NO:8; and LCDR3 comprising SEQ ID NO:10.

### Example 2: A Randomized, Double-Blind, Placebo-Controlled, Parallel Group Study to Evaluate the Efficacy and Safety of Alirocumab in Patients with Heterozygous Familial Hypercholesterolemia not Adequately Controlled with their Lipid-Modifying Therapy

### Introduction

This study included patients with heterozygous familial hypercholesterolemia (heFH) with or without a history of documented myocardial infarction (MI) or ischemic stroke.

The objective of the study was to assess the efficacy and safety of Alirocumab in patients with heFH and who require additional pharmacological management since their current lipid-modifying therapy (LMT) failed to achieve the LDL-C treatment goal.

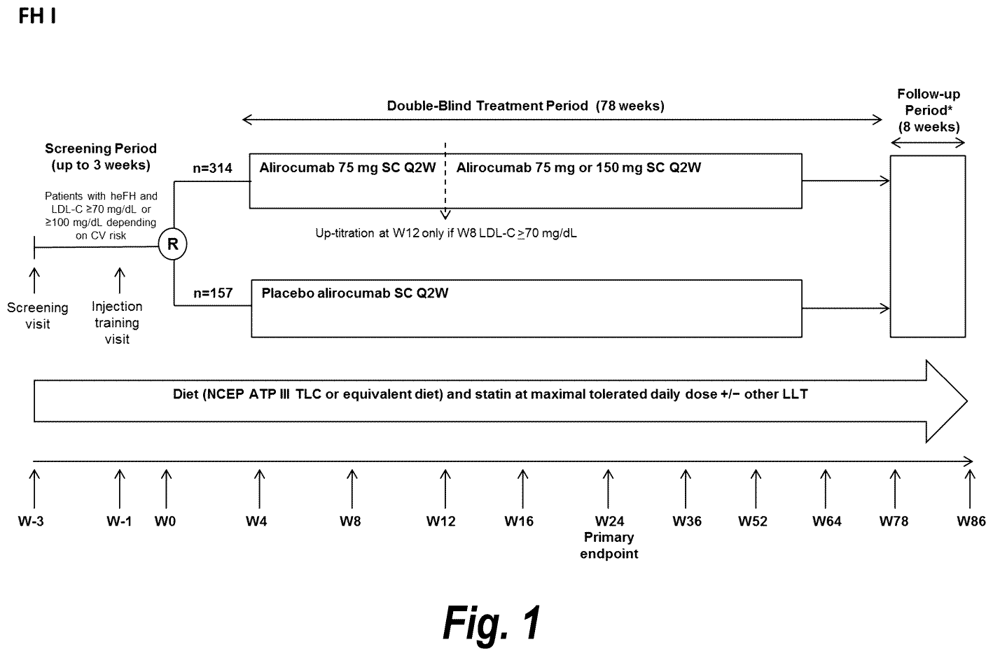

This study (FIG. 1) was undertaken to demonstrate in heFH patients who are not at their LDL-C goal that Alirocumab 75 mg Q2W or 75 mg Q2W/150 mg Q2W as add-on therapy to statin±other LMT causes a statistically significant and clinically meaningful reduction in LDL-C. This population that is not at LDL-C goal on optimized LMT represents the highest risk group with a well identified unmet medical need that can be addressed by adding Alirocumab to their LDL-C lowering therapies.

### Study Objectives

The primary objective of the study was to demonstrate the reduction of LDL-C by Alirocumab as add-on therapy to stable maximally tolerated daily statin therapy with or without other LMT in comparison with placebo after 24 weeks of treatment in patients with heFH.

The secondary objectives of the study were: 1) to evaluate the effect of Alirocumab 75 mg in comparison with placebo on LDL-C after 12 weeks of treatment; 2) to evaluate the effect of Alirocumab on other lipid parameters (i.e., Apo B, non-HDL-C, total-C, Lp (a), HDL-C, TG levels, and Apo A-1 levels); 3) to evaluate the long-term effect of Alirocumab on LDL-C; 4) to evaluate the safety and tolerability of Alirocumab; 5) to evaluate the development of anti-Alirocumab antibodies; and 6) to evaluate the PK of Alirocumab.

### Study Design

This was a randomized, double-blind, placebo-controlled, parallel-group, unbalanced (2:1, Alirocumab:placebo), multi-center, multi-national study to assess the efficacy and the safety of Alirocumab in patients with heFH not adequately controlled with their LMT (i.e., stable maximally tolerated daily statin therapy±other LMT). Not adequately controlled was defined as an LDL-C≥70 mg/dL (1.81 mmol/L) at the screening visit (Week-3) in patients with a history of documented cardiovascular disease or LDL-C≥100 mg/dL (2.59 mmol/L) at the screening visit (Week-3) in patients without a history of documented cardiovascular disease. Randomization was stratified according to prior history of MI or ischemic stroke [Yes/No], statin treatment (atorvastatin 40 to 80 mg daily or rosuvastatin 20 to 40 mg daily vs. simvastatin whatever the daily dose, atorvastatin below 40 mg daily or rosuvastatin below 20 mg daily) and geographic region. After randomization, patients received double-blind study treatment (either Alirocumab or placebo) Q2W over a period of 18 months (78 weeks) on top of stable maximally tolerated daily statin therapy±other LMT. A dose up-titration depending on Week 8 LDL-C levels may occur at Week 12 for patients randomized to Alirocumab. After completion of the 18-month double-blind treatment period, all patients who successfully completed the study had the opportunity to participate in an open-label extension study. Consequently all patients received Alirocumab at entry in the open-label extension study regardless of the study treatment they received during the 18-month double-blind treatment period.

The study consisted of 3 periods: screening, double-blind treatment, and follow-up.

The screening period was up to 3 weeks in duration including an intermediate visit during which the patient (or another designated person such as spouse, relative, etc.) was trained to self-inject/inject with placebo for Alirocumab. Eligibility assessments were performed to permit the randomization of patients into the study.

The double blind treatment period (DBTP) was a randomized, double-blind study treatment period of 18 months. The first injection during the double-blind period was done at the site on the day of randomization (Week 0 [D1]-V3). The subsequent injections were done by the patient (self-injection) or another designated person (such as spouse, relative, etc.) at a patient-preferred location (home . . . ). Patients randomized to Alirocumab received a dose of 75 mg of the Investigational Medicinal Product (IMP) from randomization (V3) up to Week 12 (V6) (i.e., Weeks 0, 2, 4, 6, 8, and 10). At the Week 12 visit (V6) these patients, in a blinded manner, either: 1) continued Alirocumab 75 mg Q2W from Week 12 onwards until the last injection at Week 76, if the Week 8 LDL-C was <70 mg/dL (1.81 mmol/L); OR 2) dose up-titrated to Alirocumab 150 mg Q2W from Week 12 onwards until the last injection at Week 76, if the Week 8 LDL-C was ≥70 mg/dL (1.81 mmol/L).

The follow-up period (if applicable) was a period of 8 weeks after the end of the DBTP for patients not consenting to participate in the open-label extension study or if prematurely discontinuing study treatment.

The laboratory measurement of lipid parameters were performed by a central laboratory (central lab) during the study.

Patients who achieved 2 consecutive calculated LDL-C levels <25 mg/dL (0.65 mmol/L) during the study were monitored and managed.

Statin and other LMT (if applicable) should be stable (including dose) during the first 24 weeks of the DBTP barring exceptional circumstances whereby overriding concerns warrant such changes. At Week 24 onwards, background LMT may be modified only under certain conditions as described below.

Patients should be on a stable diet (NCEP-ATPIII therapeutic lifestyle changes [TLC] diet or equivalent) throughout the entire study duration from screening. Table 1 provides a summary of the TLC diet for high cholesterol.

TABLE 1

| | |
|---|---|
| Total Fat | 25%-35% total calories* |
| Saturated fat* | <7% total calories |
| Polyunsaturated fat | up to 10% total calories |
| Monounsaturated fat | up to 20% total calories |
| Carbohydrates† | 50%-60% total calories* |
| Protein | ~15% total calories |
| Cholesterol | <200 mg/day (5.172 mmol/day) |
| Plant Sterols | 2 g |
| Soluble Fiber such as psyllium | 10 g-25 g |

*ATP III allows an increase of total fat to 35 percent of total calories and a reduction in carbohydrate to 50 percent for persons with the metabolic syndrome. Any increase in fat intake should be in the form of either polyunsaturated or monounsaturated fat. Trans-fatty acids are another LDL-raising fat that should be kept at a low intake.
†Carbohydrate should derive predominantly from foods rich in complex carbohydrates including grains-especially whole grains-fruits, and vegetables.

The study duration included a screening period of up to 3 weeks, a 78-week DBTP for efficacy and safety assessment, and an 8-week post-treatment follow-up period after the last visit of the DBTP for patients not consenting to participate in the open-label extension study or if prematurely discontinuing study treatment. Thus, the maximum study duration per patient was about 89 weeks (i.e., 20 months) (up to 3 weeks screening+78 weeks double-blind treatment+8 weeks follow-up). The end of the study per patient was the last protocol planned visit or the resolution/stabilization of all SAEs, and AESI, whichever came last.

### Selection of Patients

The inclusion criteria were: 1) patients with heFH* who were not adequately controlled with a maximally tolerated daily dose of statin** with or without other LMT, at stable dose prior to the screening visit (Week-3). *Diagnosis of heFH must be made either by genotyping or by clinical criteria. For those patients not genotyped, the clinical diagnosis may be based on either the Simon Broome criteria with a criteria for definite FH or the WHO/Dutch Lipid Network criteria with a score >8 points.

According to the Simon Broome Register Diagnostic Criteria for Heterozygous Familial Hypercholesterolemia, definite familial hypercholesterolemia is defined as: 1) total-C>6.7 mmol/l (260 mg/dL) or LDL cholesterol above 4.0 mmol/l (155 mg/dL) in a child <16 years or Total-C>7.5 mmol/l (290 mg/dL) or LDL cholesterol above 4.9 mmol/l (190 mg/dL) in an adult. (Levels either pre-treatment or highest on treatment); plus either A) tendon xanthomas in patient, or in 1st degree relative (parent, sibling, child), or in 2nd degree relative (grandparent, uncle, aunt); or B) DNA-based evidence of an LDL receptor mutation or familial defective Apo B.

According to the Simon Broome Register Diagnostic Criteria for Heterozygous Familial Hypercholesterolemia, possible familial hypercholesterolemia is defined as: 1) total-C>6.7 mmol/l (260 mg/dL) or LDL cholesterol above 4.0 mmol/l (155 mg/dL) in a child <16 years or Total-C>7.5 mmol/l (290 mg/dL) or LDL cholesterol above 4.9 mmol/l (190 mg/dL) in an adult. (Levels either pre-treatment or highest on treatment); and at least one of the following: A) family history of MI below 50 years of age in 2nd degree relative or below 60 years of age in 1st degree relative; and B) family history of raised cholesterols >7.5 mmol/l (290 mg/dL) in adult 1st or 2nd degree relative or >6.7 mmol/l (260 mg/dL) in child or sibling under 16 years of age.

The WHO Criteria (Dutch Lipid Network clinical criteria) for diagnosis of Heterozygous Familial Hypercholesterolemia (heFH) is set forth in Table 2.

TABLE 2
Diagnostic Scoring for Heterozygous
Familial Hypercholesterolemia
Family history

| | |
|---|---|
| a First degree relative with known premature (men <55 | 1 |
| yrs, women <60 yrs) coronary and vascular disease. | |
| b First degree relative with known LDL-cholesterol >95th | |
| percentile for age and sex. | |
| and/or | |
| a First degree relative with tendon xanthomata | 2 |
| and/or arcus cornealis. | |
| b Children below 18 yrs. with LDL-cholesterol >95th | |
| percentile for age and sex. | |

Clinical history

| | |
|---|---|
| a Patient has premature (men <55 yrs, women <60 | 2 |
| yrs) coronary artery disease | |
| b Patient has premature (men <55 yrs, women <60 | 1 |
| yrs) cerebral or peripheral vascular disease. | |

Physical examination

| | |
|---|---|
| a Tendon xanthomata | 6 |
| b Arcus cornealis below the age of 45 yrs. | 4 |

| Laboratory analysis | mmol/L | mg/dL | |
|---|---|---|---|
| a LDL-cholesterol | >8.5 | >330 | 8 |
| b LDL-cholesterol | 6.5-8.4 | 250-329 | 5 |
| c LDL-cholesterol | 5.0-6.4 | 190-249 | 3 |
| d LDL-cholesterol | 4.0-4.9 | 155-189 | 1 |

(HDL-cholesterol and triglycerides are normal)

| DNA-analysis | |
|---|---|
| a Functional mutation low-density lipoprotein receptor gene present | 8 |

Diagnosis of heFH is:

| | |
|---|---|
| Certain When | >8 points |
| Probable When | 6-8 points |
| Possible When | 3-5 points |

**Definition of maximally tolerated dose (any of the following were acceptable): 1) rosuvastatin 20 mg or 40 mg daily; 2) atorvastatin 40 mg or 80 mg daily; 3) simvastatin 80 mg daily (if already on this dose for >1 year); or 4) patients not able to be on any of the above statin doses, should be treated with the dose of daily atorvastatin, rosuvastatin or simvastatin that is considered appropriate for the patient as per the investigator's judgment or concerns. Some examples of acceptable reasons for a patient taking a lower statin dose included, but were not limited to: adverse effects on higher doses, advanced age, low body mass index, regional practices, local prescribing information, concomitant medications, co-morbid conditions such as impaired glucose tolerance/impaired fasting glucose.

Patients who met all of the above inclusion criteria were screened for the following exclusion criteria, which are sorted and numbered in the following 3 subsections: exclusion criteria related to study methodology, exclusion criteria related to the active comparator and/or mandatory background therapies, and exclusion criteria related to Alirocumab.

Exclusion criteria related to study methodology were: 1) patient without diagnosis of heFH made either by genotyping or by clinical criteria; 2) LDL-C<70 mg/dL (<1.81 mmol/L) at the screening visit (Week-3) and patient with history of documented cardiovascular disease. Cardiovascular disease was defined as coronary heart disease, ischemic stroke or peripheral arterial disease; 3) LDL-C<100 mg/dL (<2.59 mmol/L) at the screening visit (Week-3) and patient without history of documented cardiovascular disease; 4) not on a stable dose of LMT (including statin) for at least 4 weeks and/or fenofibrate for at least 6 weeks, as applicable, prior to the screening visit (Week −3) and from screening to randomization; 5) currently taking a statin other than simvastatin, atorvastatin, or rosuvastatin; 6) simvastatin, atorvastatin, or rosuvastatin is not taken daily or not taken at a registered dose; 7) daily doses above atorvastatin 80 mg, rosuvastatin 40 mg, or simvastatin 40 mg (except for patients on simvastatin 80 mg for more than one year, who are eligible); 8) use of fibrates, other than fenofibrate within 6 weeks of the screening visit (Week-3) or between screening and randomization visits; 9) use of nutraceutical products or over-the-counter therapies that may affect lipids which have not been at a stable dose/amount for at least 4 weeks prior to the screening visit (Week −3) or between screening and randomization visits; 10) use of red yeast rice products within 4 weeks of the screening visit (Week-3) or between screening and randomization visits; 11) patient who has received plasmapheresis treatment within 2 months prior to the screening visit (Week −3), or has plans to receive it during the study; 12) recent (within 3 months prior to the screening visit [Week −3] or between screening and randomization visits) MI, unstable angina leading to hospitalization, percutaneous coronary intervention (PCI), coronary artery bypass graft surgery (CABG), uncontrolled cardiac arrhythmia, stroke, transient ischemic attack (TIA), carotid revascularization, endovascular procedure or surgical intervention for peripheral vascular disease; 13) planned to undergo scheduled PCI, CABG, carotid, or peripheral revascularization during the study; 14) systolic BP>160 mmHg or diastolic BP>100 mmHg at screening visit or randomization visit; 15) history of New York Heart Association (NYHA) Class III or IV heart failure within the past 12 months; 16) known history of a hemorrhagic stroke; 17) age <18 years or legal age of majority at the screening visit (Week-3), whichever is greater; 18) patients not previously instructed on a cholesterol-lowering diet prior to the screening visit (Week-3); 19) newly diagnosed (within 3 calendar months prior to randomization visit [Week 0]) or poorly controlled (glycated haemoglobin A1c [HbA_{1c}]>9% at the screening visit [Week-3]diabetes); 20) presence of any clinically significant uncontrolled endocrine disease known to influence serum lipids or lipoproteins. Note that patients on thyroid replacement therapy can be included if the dosage has been stable for at least 12 weeks prior to screening and between screening and randomization visits, and TSH level is within the normal range of the Central Laboratory at the screening visit; 21) history of bariatric surgery within 12 months prior to the screening visit (Week-3); 22) unstable weight defined by a variation >5 kg within 2 months prior to the screening visit (Week-3); 23) known history of homozygous FH; 24) known history of loss of function of PCSK9 (i.e., genetic mutation or sequence variation); 25) use of systemic corticosteroids, unless used as replacement therapy for pituitary/adrenal disease with a stable regimen for at least 6 weeks prior to randomization visit (Week 0). Note that topical, intra-articular, nasal, inhaled and ophthalmic steroid therapies were not considered as ‘systemic’ and were allowed; 26) use of continuous estrogen or testosterone hormone replacement therapy unless the regimen has been stable in the past 6 weeks prior to the Screening visit (Week-3) and no plans to change the regimen during the study; 27) history of cancer within the past 5 years, except for adequately treated basal cell skin cancer, squamous cell skin cancer or in situ cervical cancer; 28) known history of a positive HIV test; 29) patient who has taken any investigational drugs other than the Alirocumab training placebo kits within 1 month or 5 half lives, whichever is longer; 30) patient who has been previously treated with at least one dose of Alirocumab or any other anti-PCSK9 monoclonal antibody in other clinical trials; 31) patient who withdraws consent during the screening period (patient who is not willing to continue or fails to return); 32) conditions/situations such as: a) any clinically significant abnormality identified at the time of screening that, in the judgment of the Investigator or any sub-Investigator, would preclude safe completion of the study or constrain endpoints assessment; e.g., major systemic diseases, patients with short life expectancy; or b) considered by the Investigator or any sub-Investigator as inappropriate for this study for any reason, e.g.: deemed unable to meet specific protocol requirements, such as scheduled visits; deemed unable to administer or tolerate long-term injections as per the patient or the Investigator; Investigator or any sub-Investigator, pharmacist, study coordinator, other study staff or relative thereof directly involved in the conduct of the protocol, etc; presence of any other conditions (eg, geographic or social), either actual or anticipated, that the Investigator feels would restrict or limit the patient's participation for the duration of the study; or 33) laboratory findings during screening period (not including randomization Week 0 labs): positive test for Hepatitis B surface antigen or Hepatitis C antibody; positive serum beta-hCG or urine pregnancy test (including Week 0) in women of childbearing potential (WOCBP); triglycerides >400 mg/dL (>4.52 mmol/L) (1 repeat lab is allowed); estimated glomerular filtration rate (eGFR)<30 mL/min/1.73 m2 according to 4-variable modification of diet in renal disease (MDRD) Study equation (calculated by central lab); alanine aminotransferase (ALT) or aspartate aminotransferase (AST) >3×upper limit of normal range (ULN) (1 repeat lab is allowed); CPK>3×ULN (1 repeat lab is allowed); TSH<lower limit of normal (LLN) or >ULN (1 repeat lab is allowed).

Exclusion criteria related to the active comparator and/or mandatory background therapies were: 1) all contraindications to the background therapies or warnings/precautions of use (when appropriate) as displayed in the respective National Product Labeling.

Exclusion criteria related to Alirocumab were: 1) known hypersensitivity to monoclonal antibody or any component of the drug product; 2) pregnant or breast-feeding women; or 3) women of childbearing potential not protected by highly-effective method(s) of birth control (as defined in the informed consent form and/or in a local protocol addendum) and/or who are unwilling or unable to be tested for pregnancy. Note that women of childbearing potential must have a confirmed negative pregnancy test at screening and randomization visits. They must use an effective contraceptive method throughout the entire duration of the study treatment, and for 10 weeks after the last intake of IMP, and agree to repeat urine pregnancy test at designated visits. Postmenopausal women must be amenorrheic for at least 12 months.

Coronary heart disease, ischemic stroke, and peripheral arterial disease, as defined in exclusion criteria number 2 related to study methodology was as follows. Documented history of CHD (includes one or more of the following): acute myocardial infarction (MI); silent myocardial infarction; unstable angina; coronary revascularization procedure (eg, percutaneous coronary intervention [PCI] or coronary artery bypass graft surgery [CABG]); clinically significant CHD diagnosed by invasive or non-invasive testing (such as coronary angiography, stress test using treadmill, stress echocardiography or nuclear imaging).

Documented previous ischemic stroke with a focal ischemic neurological deficit that persisted more than 24 hours, considered as being of atherothrombotic origin. CT or MRI must have been performed to rule out hemorrhage and non-ischemic neurological disease.

Documented peripheral arterial disease (one of the following criteria must be satisfied): 1) current intermittent claudication (muscle discomfort in the lower limb produced by exercise that is both reproducible and relieved by rest within 10 minutes) of presumed atherosclerotic origin together with ankle-brachial index equal to or less than 0.90 in either leg at rest or 2) history of intermittent claudication (muscle discomfort in the lower limb produced by exercise that is both reproducible and relieved by rest within 10 minutes) together with endovascular procedure or surgical intervention in one or both legs because of atherosclerotic disease or 3) history of critical limb ischemia together with thrombolysis, endovascular procedure or surgical intervention in one or both legs because of atherosclerotic disease.

### Study Treatments

Sterile Alirocumab drug product was supplied at a concentration of 75 mg/mL and 150 mg/mL both as 1 mL volume in an auto-injector. The drug substance was formulated in histidine, pH 6.0, polysorbate 20, and sucrose.

Sterile placebo for Alirocumab was prepared in the same formulation as Alirocumab without the addition of protein as 1 mL volume in an auto-injector.

During the double-blind treatment period, Alirocumab or placebo was administered subcutaneously Q2W, starting at Week 0 continuing up to the last injection (Week 76) 2 weeks before the end of the double blind treatment period (DBTP). If the injection was scheduled to take place on the same date as the site visit, then the IMP was administered after the blood sampling was completed.

Investigational Medicinal Product (IMP) should ideally have been administered Q2W subcutaneously at approximately the same time of the day; however it was acceptable to have a window period of ±3 days. The time of the day was based on the patient's preference.

The following classes of drugs were identified as non-NIMP because the medication was either a background therapy or a potential rescue medication: statins (rosuvastatin, atorvastatin, simvastatin); cholesterol absorption inhibitors (ezetimibe); bile acid-binding sequestrants (such as cholestyramine, colestipol, colesevelam); nicotinic acid; fenofibrate; and omega-3 fatty acids (≥1000 mg daily).

Patients were randomized to receive either placebo or Alirocumab during the double-blind study treatment period using a ratio 1:2, with permuted-block randomization. Randomization was stratified according to prior history of MI or ischemic stroke [Yes/No], statin treatment (atorvastatin 40 to 80 mg daily or rosuvastatin 20 to 40 mg daily vs. simvastatin whatever the daily dose, atorvastatin below 40 mg daily or rosuvastatin below 20 mg daily) and geographic region.

A concomitant medication was any treatment received by the patient concomitantly to the study (until follow-up visit). Concomitant medications should be kept to a minimum during the study. However, if these are considered necessary for the patient's welfare and are unlikely to interfere with the IMP, they may be given at the discretion of the Investigator, with a stable dose (when possible). Besides the specific information related to concomitant medications provided in this section, any other concomitant medication(s) will be allowed. If the patient has an LDL-C≥160 mg/dL (4.14 mmol/L) at the screening visit (Week-3) and is treated with a statin only, i.e., without additional LMT, the investigator will have to report the reason for the patient not being on a second LMT. For background LMT, including statins, sites must follow the national product label for the safety monitoring and management of patients.

Nutraceutical products or over-the-counter therapies that may affect lipids were allowed only if they had been used at a stable dose for at least 4 weeks prior to screening visit, during the screening period and maintained during the first 24 weeks of the double-blind treatment period. After the Week 24 visit, modification to these nutraceutical products or over-the-counter therapies was allowed but in general should be avoided. Examples of such nutraceutical products or over-the-counter therapies include omega-3 fatty acids at doses <1000 mg, plant stanols such as found in Benecol, flax seed oil, and psyllium.

Patients must have been on stable maximally tolerated daily registered doses of statins with or without other LMT for at least 4 weeks (6 weeks for fenofibrate) before screening visit. During the study, the patients should stay on these stable maximally tolerated registered daily doses of statins with or without other LMT. From the screening visit (Week-3) until Week 24 of the double-blind treatment period, the background LMT should not be changed. No dose adjustment, discontinuation or initiation of other statins or other LMT should take place during this time, barring exceptional circumstances whereby overriding concerns (including but not limited to triglyceride alert posted by the central lab) warrant such changes, as per the Investigator's judgment.

For a rescue notification of LDL-C at the Week 24 visit and later, i.e., LDL-C increase >25% as compared to randomization visit LDL-C on two consecutive occasions, the Investigator should have ensured that no reasonable explanation existed for insufficient LDL-C control (such as an alternative medical cause like corticosteroid use, etc) and in particular that: compliance with diet was appropriate; compliance with background LMT was appropriate; and study treatment was given as planned. If any of the above could reasonably explain the insufficient LDL-C control, the Investigator should have undertaken appropriate action, i.e., stress on the absolute need to be compliant with treatment, if needed organize a specific interview with a qualified nutrition professional and stress on the absolute need to be compliant with diet, and perform a blinded LDL-C assessment within 1 to 2 months. If none of the above mentioned reasons were found, or if appropriate action failed to decrease LDL-C under the alert value, rescue medication may have been introduced.

If no reason for LDL-C above the threshold value could be found, or if appropriate action failed to decrease LDL-C below the threshold value, rescue medication may have been introduced. The effectiveness of any such changes was to be made based on lack of rescue threshold from blinded lipid testing at the next routinely scheduled lab draw. Patients per protocol already received a maximum tolerated dose of statin, so statin uptitration or switch was not an option. For further LDL-C lowering, the investigator could consider adding: a cholesterol absorption inhibitor (ezetimibe), or a bile acid-binding sequestrant (the resins cholestyramine and colestipol, or colesevelam, a nonabsorbable polymer). The following lipid-modifying agents could also be considered: fibrate (Note: Caution should be exercised when combining fibrates with other cholesterol-lowering medications such as statins because of the risk of myopathy. When a fibrate is combined with a statin, fenofibrate is the fibrate of choice because it does not affect statin glucuronidation. The only fibrate allowed per protocol was fenofibrate); nicotinic acid (niacin) (Note: Niacin raises blood glucose but has been shown to be effective in modifying lipid disorders in people with diabetes if glucose control is maintained).

In summary, background LMT should not be modified from screening to the follow-up visit. However, up to Week 24, if a confirmed TG alert was reached or if there was an overwhelming clinical concern (at the discretion of the Investigator) then modification of the background LMT was allowed. At Week 24 onwards, if a confirmed TG alert was reached, or if a rescue threshold for LDL-C was attained (and no other reasonable explanation exists), or if there was an overwhelming clinical concern (at the discretion of the Investigator) then modification of the background LMT was allowed.

Women of childbearing potential must take an effective contraceptive method throughout the study treatment and for 10 weeks after the last IMP injection (e.g., Follow-up visit).

Forbidden concomitant medications from the initial screening visit until the follow-up visit included the following: statins other than simvastatin, atorvastatin and rosuvastatin; fibrates, other than fenofibrate; and red yeast rice products.

### Study Endpoints

The primary efficacy endpoint was the percent change in calculated LDL-C from baseline to Week 24, which was defined as: 100× (calculated LDL-C value at Week 24−calculated LDL-C value at baseline)/calculated LDL-C value at baseline. The baseline calculated LDL-C value was the last LDL-C level obtained before the first double-blind IMP injection. The calculated LDL-C at Week 24 was the LDL-C level obtained within the Week 24 analysis window and during the main efficacy period. The main efficacy period was defined as the time from the first double-blind IMP injection up to 21 days after the last double-blind IMP injection or up to the upper limit of the Week 24 analysis window, whichever came first. All calculated LDL-C values (scheduled or unscheduled, fasting or not fasting) may be used to provide a value for the primary efficacy endpoint if appropriate according to above definition.

The key secondary efficacy endpoints were: 1) the percent change in calculated LDL-C from baseline to Week 12: similar definition and rules as for primary efficacy endpoint, except that the calculated LDL-C at Week 12 was the LDL-C level obtained within the Week 12 analysis window and during the 12-week efficacy period. The 12-week efficacy period was defined as the time from the first double-blind IMP injection up to the Visit 6 re-supply IVRS contact or up to 21 days after the last double-blind IMP injection, whichever came first. Blood sampling collected the day of the Visit 6 re-supply IVRS contact was considered as before titration; 2) the percent change in Apo B from baseline to Week 24, using the same definition and rules as for the primary endpoint; 3) the percent change in non-HDL-C from baseline to Week 24, using the same definition and rules as for the primary endpoint; 4) the percent change in total-C from baseline to Week 24, using the same definition and rules as for the primary endpoint; 5) the percent change in Apo B from baseline to Week 12, using the same definition and rules as for the percent change in calculated LDL-C from baseline to Week 12; 6) the percent change in non-HDL-C from baseline to Week 12, using the same definition and rules as for the percent change in calculated LDL-C from baseline to Week 12; 7) the percent change in total-C from baseline to Week 12, using the same definition and rules as for the percent change in calculated LDL-C from baseline to Week 12; 8) the percent change in calculated LDL-C from baseline to Week 52, using definitions and rules that were similar to the ones used for the primary endpoint replacing Week 24 by Week 52. Note that the 52-week efficacy period was defined as the time from the first double-blind IMP injection up to 21 days after the last double-blind IMP injection, or up to the upper limit of the Week 52 analysis window, whichever came first; 9) the proportion of patients reaching LDL-C goal at Week 24, i.e., LDL-C<70 mg/dL (1.81 mmol/L) in case of prior CVD or <100 mg/dL (2.59 mmol/L) for patients without prior CVD, defined as: (number of patients whose calculated LDL-C value at Week 24 reach LDL-C goal/number of patients in the modified intent-to-treat (mITT) population)*100, using definition and rules used for the primary endpoint; 10) the proportion of patients reaching LDL-C<70 mg/dL (1.81 mmol/L) at Week 24; 11) the percent change in Lp(a) from baseline to Week 24, using the same definition and rules as for the primary endpoint; 12) the percent change in HDL-C from baseline to Week 24, using the same definition and rules as for the primary endpoint; 13) the percent change in HDL-C from baseline to Week 12, using the same definition and rules as for the percent change in calculated LDL-C from baseline to Week 12; 14) the percent change in Lp(a) from baseline to Week 12, using the same definition and rules as for the percent change in calculated LDL-C from baseline to Week 12; 15) the percent change in fasting TG from baseline to Week 24, using the same definition and rules as for the primary endpoint; 16) the percent change in fasting TG from baseline to Week 12, using the same definition and rules as for the percent change in calculated LDL-C from baseline to Week 12; 17) the percent change in Apo A-1 from baseline to Week 24, using the same definition and rules as for the primary endpoint; and 18) the percent change in Apo A-1 from baseline to Week 12, using the same definition and rules as for the percent change in calculated LDL-C from baseline to Week 12.

Other secondary efficacy endpoints were: 1) the percent change in calculated LDL-C from baseline to Week 78, using definitions and rules that were similar to the ones used for the primary endpoint replacing Week 24 by Week 78. The 78-week efficacy period was defined as the time from the first double-blind IMP injection up to 21 days after the last double-blind IMP injection, or up to the upper limit of the Week 78 analysis window, whichever came first; 2) the proportion of patients reaching LDL-C goal at Weeks 12, 52, and 78, i.e., LDL-C<70 mg/dL (1.81 mmol/L) in case of prior CVD or <100 mg/dL (2.59 mmol/L) for patients without prior CVD; 3) the proportion of patients reaching LDL-C<100 mg/dL (2.59 mmol/L) at Week 24; 4) the proportion of patients reaching LDL-C<100 mg/dL (2.59 mmol/L) at Week 12; 5) the proportion of patients reaching LDL-C<70 mg/dL (1.81 mmol/L) at Week 12; 6) the absolute change in calculated LDL-C (mg/dL and mmol/L) from baseline to Weeks 12, 24, 52, and 78; 7) the percent change in Apo B, non-HDL-C, total-C, Lp (a), HDL-C, fasting TG, and Apo A-1 from baseline to Weeks 52 and 78; 8) the change in ratio Apo B/Apo A-1 from baseline to Weeks 12, 24, 52, and 78; 9) the proportion of patients with Apo B<80 mg/dL (0.8 g/L) at Weeks 12, 24, 52, and 78; 10) the proportion of patients with non-HDL-C<100 mg/dL at Weeks 12, 24, 52, and 78; and 11) the proportion of patients with calculated LDL-C<70 mg/dL (1.81 mmol/L) and/or ≥50% reduction in calculated LDL-C (if calculated LDL-C≥70 mg/dL [1.81 mmol/L]) at Weeks 12, 24, 52, and 78.

Other endpoints were: anti-Alirocumab antibody assessments, high-sensitivity C-reactive protein, glycated haemoglobin A1c, EQ-5D Questionnaire, pharmacogenetics, and pharmacokinetics. Anti-Alirocumab antibodies included the antibody status (positive/negative) and antibody titers. Serum samples for anti-Alirocumab antibody determination were drawn periodically throughout the study. The first scheduled sample at randomization visit was obtained before IMP injection (predose). Patients who had a titer at or above 240 for anti-Alirocumab antibody at follow-up visit had additional antibody sample(s), at 6 to 12 months after the last dose and thereafter, about every 3 to 6 months until titer returns below 240. The percent change in high-sensitivity C-reactive protein (hs-CRP) was measured at baseline and Weeks 24, 52, and 78. EQ-5D is a standardized measure of health status developed by the EuroQol Group in order to provide a simple, generic measure of health for clinical and economic appraisal. The EQ-5D as a measure of health-related quality of life defines health in terms of 5 dimensions: mobility, self-care, usual activities, pain/discomfort, anxiety/depression. Each dimension can take one of three responses (3 ordinal levels of severity): “no problem” (1); “some problems” (2); “severe problems” (3); Overall health state is defined as a 5-digit number. Health states defined by the 5-dimensional classification can be converted into corresponding index scores that quantify health status, where 0 represents ‘death’ and 1 represents “perfect health”.

### Study Procedures

For all visits after Day 1/Week 0 (randomization visit), a timeframe of a certain number of days was allowed. The window period for visits at Weeks 12 and 24 were ±3 days, at Weeks 52 and 78 was ±5 days, and for all other site visits it was ±7 days during the double-blind treatment period, and follow-up period. A window period of +3 days was allowed for the randomization visit (Day1/Week 0) and ±7 days for the injection training visit during the screening period (Week-1). For all visits after Day 1/randomization visit, if one visit date is changed, then the next visit should take place according to the original schedule.

### Safety

Occurrence of treatment emergent adverse events (TEAEs) reported by the patient or noted by the investigator, serious adverse events (SAEs), TEAEs leading to treatment discontinuation, AEs of special interest (local Injection site reactions, allergic events, selected neurological events and cardiovascular events confirmed by adjudication result), occurrence of PCSA (potentially clinically significant abnormalities) in laboratory parameters, exploratory analysis for patients with 2 consecutive calculated LDL-C<25 mg/dL (<0.65 mmo/L) and for changes in blood glucose control, including diabetes.

### Statistical Methods

### Sample Size Determination:

A total sample size of 45 patients (30 in alirocumab and 15 in placebo) had 95% power to detect a difference in mean percent change in LDL-C of 30% with a 0.05 two-sided significance level and assuming a common standard deviation of 25%, and all these 45 patients having an evaluable primary endpoint. Nevertheless, to meet regulatory requirements across the program, the sample size was increased to assess the safety of alirocumab. In order to have at least 225 patients on alirocumab followed for 12 months in this study, and assuming a drop-out rate of 10% over the first 3-month period and a drop-out rate of 20% over the remaining 9-month period, the final total sample size was increased to 471 with a randomization ratio 2:1 (alirocumab 314: placebo 157).

### Timing of Analyses:

The first step analysis included efficacy endpoints up to Week 52 (final efficacy analysis) and interim safety analysis, which was performed on all safety data up to the common study cut-off date (last patient Week 52 visit). Analysis of lipid data beyond Week 52 was descriptive. These results are presented herein.

The second step (final) analysis will be conducted at the end of the study and will consist in the final analysis of efficacy endpoints up to Week 78 and final safety analysis.

### Analysis Populations:

The primary efficacy analysis population was the intent-to-treat (ITT) population, defined as all randomized patients who had an evaluable primary endpoint, that is, those with an available baseline calculated LDL-C value, and at least one available calculated LDL-C value within one of the analysis windows up to Week 24 (including all calculated LDL-C values on-treatment and off-treatment).

The secondary efficacy analysis population was the modified intent-to-treat (mITT) population, defined as all randomized patients who took at least one dose or part of a dose of the double-blind investigational medicinal product (IMP) and who had an available calculated LDL-C value at baseline and at least one within one of the analysis windows up to Week 24 during the efficacy treatment period. The efficacy treatment period was defined as the time from the first double-blind IMP injection up to 21 days after the last double-blind injection.

The safety population included all randomized patients who received at least one dose or part of a dose of the double-blind IMP.

### Efficacy Analyses:

Primary analyses of efficacy endpoints were conducted using an ITT approach (based on the ITT population defined above), including all lipid data, regardless of whether the patient was continuing therapy or not. This corresponds to ITT estimands, defined for primary and key secondary endpoints. In addition, analyses were also conducted using an on-treatment approach (based on the mITT population defined above), including lipid data collected during the efficacy treatment period. This corresponds to on-treatment estimands of key secondary endpoints.

The ITT approach analyzed all patients, irrespective of their adherence to the treatment; it assessed the benefit of the treatment strategy and reflected as much as possible the effect in a population of patients. The on-treatment approach analyzed the effect of treatment, restricted to the period during which patients actually received the treatment. It assessed the benefit that a treatment would achieve in patients adherent to treatment up to the considered time point.

Efficacy analyses were performed according to treatment as-randomized.

All measurements, scheduled or unscheduled, fasting or not fasting, were assigned to analysis windows in order to provide an assessment for Week 4 to Week 78 time points.

With regards to the primary efficacy analysis (ITT approach), the percent change in calculated LDL-C from baseline to Week 24 was analyzed using a mixed-effect model with repeated measures (MMRM) approach. All post-baseline data available from Week 4 to Week 52 analysis windows were used and missing data were accounted for by the MMRM. The model included the fixed categorical effects of treatment group (placebo versus alirocumab), randomization strata (as per IVRS), time point (Week 4 to Week 52), treatment-by-time point interaction and strata-by-time point interaction, as well as the continuous fixed covariates of baseline LDL-C value and baseline value-by-time-point interaction. This model provided baseline adjusted least-squares means (LSmeans) estimates at Week 24 for both treatment groups with their corresponding standard errors and 95% confidence intervals. To compare the alirocumab to the placebo group, an appropriate contrast statement was used to test the differences of these estimates at the 5% alpha level.

A hierarchical procedure was defined to test key secondary endpoints while controlling for multiplicity (using above order of key secondary endpoints). The first key secondary endpoint was the percent change in calculated LDL-C from baseline to Week 24 using an on-treatment approach.

Continuous secondary variables anticipated to have a normal distribution (i.e., lipids other than TGs and Lp(a)) were analyzed using the same MMRM model as for the primary endpoint. Continuous endpoints anticipated to have a non-normal distribution (i.e., TGs and Lp(a)) were analyzed using multiple imputation approach for handling of missing values followed by robust regression model with endpoint of interest as response variable using M-estimation (using SAS ROBUSTREG procedure) with treatment group, randomization strata (as per IVRS) and corresponding baseline value(s) as effects to compare treatment effects. Combined estimate for mean in both treatment groups, as well as the differences of these estimates, with their corresponding SEs, 95% CIs and p-value were provided (through SAS MIANALYZE procedure).

Binary secondary efficacy endpoints were analyzed using multiple imputation approach for handling of missing values followed by stratified logistic regression with treatment group as main effect and corresponding baseline value(s) as covariate, stratified by randomization factors (as per IVRS). Combined estimates of odds ratio versus placebo, 95% CI, and p-value were provided (through SAS MIANALYZE procedure).

### Safety Analyses:

Safety analyses were descriptive, performed on the safety population according to treatment actually received. The safety analysis focused on the TEAE period defined as the time from the first dose of double-blind IMP up to 70 days after the last double-blind injection. TEAE which developed, worsened or became serious or PCSA occurring after the patient inclusion in the open-label extension study (LTS13643) were not considered in the TEAE period. TEAE period was truncated at the common study cut-off date.

### Results

### Study Patients

### Patient Accountability

Of the 486 randomized patients (323 patients and 163 patients in the alirocumab and the placebo groups, respectively), one patient in the alirocumab group was not treated and was therefore not included in the safety population. This patient was also excluded from the ITT population (no LDL-C value within one of the analysis windows up to Week 24 as the patient withdrew consent on Day 1).

Two randomized patients in the alirocumab group were excluded from the mITT population (one patient excluded from the ITT population and one patient with no LDL-C value within one of the analysis windows up to Week 24 during the efficacy treatment period).

TABLE 3
Analysis populations

| | | Alirocumab 75 | |
|---|---|---|---|
| | | Q2W/Up150 | |
| | Placebo | Q2W | All |
| Randomized population | 163 (100%) | 323 (100%) | 486 (100%) |
| Efficacy populations | | | |
| Intent-to-Treat (ITT) | 163 (100%) | 322 (99.7%) | 485 (99.8%) |
| Modified Intent-to-Treat | 163 (100%) | 321 (99.4%) | 484 (99.6%) |
| (mITT) | | | |
| Safety population | 163 | 322 | 485 |

Note:
The safety population patients are tabulated according to treatment actually received (as treated).
For the other populations, patients are tabulated according to their randomized treatment.

In the alirocumab group, among the 311 patients who received at least one injection after Week 12, 135 (43.4%) patients received automatic up-titration at Week 12 from alirocumab 75 mg Q2W to 150 mg Q2W in a blinded manner.

### Study Disposition

Study disposition, exposure and safety analyses were assessed using all data up to the study common cut-off date (defined as the date of the last patient's Week 52 visit). Therefore, this first step analysis includes data beyond Week 52 and up to Week 78 or Follow-up visit for some patients.

There were in total 7 (1.4%) randomized patients who completed the 78-week double-blind study treatment period and 424 (87.2%) randomized patients with treatment ongoing at the time of the first-step analysis cut-off date. The double-blind IMP was prematurely discontinued before Week 78 for 18 (11.0%) randomized patients in the placebo group and 36 (11.1%) randomized patients in the alirocumab group. The main reasons for study treatment discontinuation were adverse event and other reasons.

In addition, among these patients 34 (10.5%) randomized patients had prematurely discontinued the double-blind IMP before the Week 52 visit in the alirocumab group and 15 (9.2%) patients in the placebo group.

In this first step analysis, final results are available for the primary efficacy endpoint at Week 24 and key secondary efficacy endpoints were assessed at Week 12, Week 24 and Week 52. The primary endpoint was missing for 46 patients at the week 24 visit for the following reasons: 18 samples were not done due to earlier study discontinuation, 14 samples were done outside the analysis time window, 4 missing samples while visit Week 24 was done, and 10 samples were done but the measurement could not be done (lipemia, insufficient quantity, TGs >400 mg/dL[>4.52 mmol/L], sample lost, . . . ).

### Demographics, Baseline, and Summary Population Characteristics

Demographic characteristics, disease characteristics and lipid parameters at baseline were similar in the alirocumab group as compared to the placebo group (see Table 4). 486 heFH patients diagnosed by genotyping (39%) or WHO or Simon Broome criteria (61%) were randomized (2:1) to alirocumab (75 mg Q2W potentially uptitrated to 150 mg Q2W) or placebo (323 versus 163, respectively). Half of the randomized population (51%) had a history of at least one coronary heart disease (CHD) or multiple CHD risk factors that defined these patients being at very high cardiovascular risk. Demographics characteristics, disease characteristics and lipid parameters at baseline were similar in the alirocumab group as compared to the placebo group. All patients were treated with a statin, 82% receiving a dose defined as high intensity statin (atorvastatin 40 to 80 mg daily or rosuvastatin 20 to 40 mg daily) and 57% receiving ezetimibe in addition to the statin. Mean (SD) calculated LDL-C at baseline was 144.6 (49.7) mg/dL [3.75 (1.29) mmol/L].

Exposure to injections was similar across treatment groups with a mean exposure of 59 weeks. In the alirocumab group, among the 311 patients who received at least one injection after Week 12, 135 (43.4%) patients received automatic up-titration at Week 12 from alirocumab 75 mg Q2W to 150 mg Q2W in a blinded manner.

TABLE 4
Baseline Characteristics of FHI Patient Population

| | Alirocumab | | Placebo | |
|---|---|---|---|---|
| Characteristic | (N = 323) | | (N = 163) | |
| Diagnosis of heFH^{†}, % (n) | | | | |
| Genotyping | 39.9% | (129) | 38.0% | (62) |
| Clinical criteria | 59.8% | (193) | 62.0% | (101) |
| Age, mean (SD), yrs | 52.1 | (12.9) | 51.7 | (12.3) |
| Male | 55.7% | (180) | 57.7% | (94) |
| Race, white | 92.9% | (300) | 88.3% | (144) |
| BMI, mean (SD), kg/m² | 29.0 | (4.6) | 30.0 | (5.4) |
| CHD history | 45.5% | (147) | 47.9% | (78) |
| CHD risk equivalents^{†} | 16.7% | (54) | 15.3% | (25) |
| Current smoker | 12.1% | (39) | 18.4% | (30) |
| Hypertension | 43.0% | (139) | 43.6% | (71) |
| Type 2 diabetes | 9.6% | (31) | 15.3% | (25) |

% (N) of patients unless stated All pts on background of max tolerated statin ± other lipid-lowering therapy.
^{†}Diagnosis of heFH must be made either by genotyping or by clinical criteria. For those patients not genotyped, the clinical diagnosis may be based on either the Simon Broome criteria for definite FH or the WHO/Dutch Lipid Network criteria with a score of >8 points. In FH I, one patient was categorised as “probable” FH by clinical criteria - genotyping results for this patient are pending.

TABLE 5
Disease characteristics and other relevant baseline data - Randomized population

| | | |
|---|---|---|
| | Alirocumab 75 | |
| Placebo | Q2W/Up150 Q2W | All |
| (N = 163) | (N = 323) | (N = 486) |

Type of hypercholesterolemia

| | | | |
|---|---|---|---|
| Heterozygous Familial Hypercholesterolemia (heFH) | 163 (100%) | 323 (100%) | 486 (100%) |
| Non-Familial Hypercholesterolemia (non-FH) | 0 | 0 | 0 |

Time from hypercholesterolemia diagnosis (years)

| | | | |
|---|---|---|---|
| Number | 163 | 323 | 486 |
| Mean (SD) | 13.28 (11.38) | 12.19 (11.38) | 12.55 (11.38) |
| Median | 9.43 | 8.82 | 9.03 |
| Min:Max | 0.0:42.6 | 0.0:60.7 | 0.0:60.7 |

Confirmation of diagnosis

| | | | |
|---|---|---|---|
| By genotyping | 62 (38.0%) | 129 (39.9%) | 191 (39.3%) |
| By WHO/Simon Broome^{a} | 101 (62.0%) | 193 (59.8%) | 294 (60.5%) |

^{a}for heFH diagnosis not confirmed by genotyping.
Note:
at time of screening, one patient was included based on clinical criteria with a score of 8 for the WHO criteria. As the clinical score characterized the patient as probable heFH rather than certain, a genotyping was performed to confirm heFH status but these results are still pending.

TABLE 6
Cardiovascular History and Risk Factors Breakdown

| | Alirocumab | | Placebo | |
|---|---|---|---|---|
| Characteristic | (N = 323) | | (N = 163) | |
| CHD history | 45.5% | (147) | 47.9% | (78) |
| Acute MI | 22.0% | (71) | 26.4% | (43) |
| Silent MI | 2.5% | (8) | 1.2% | (2) |
| Unstable angina | 11.1% | (36) | 15.3% | (25) |
| Coronary revasc. | 31.6% | (102) | 34.4% | (56) |
| Other clinically significant CHD | 26.9% | (87) | 29.4% | (48) |
| CHD risk equivalents | 16.7% | (54) | 15.3% | (25) |
| Ischemic stroke | 4.0% | (13) | 1.8% | (3) |
| Peripheral arterial disease | 2.8% | (9) | 2.5% | (4) |
| Moderate CKD | 6.2% | (20) | 5.5% | (9) |
| Diabetes + 2 or more risk factors | 5.9% | (19) | 6.1% | (10) |

% (N) of patients unless stated. All pts on background of max tolerated statin ± other lipid-lowering therapy

TABLE 7
Background LMT at randomization - Randomized population

| | | | Alirocumab 75 | | | |
|---|---|---|---|---|---|---|
| | Placebo | | Q2W/Up150 Q2W | | All | |
| | (N = 163) | | (N = 323) | | (N = 486) | |
| Any statin | 163 | (100%) | 323 | (100%) | 486 | (100%) |
| Taking high intensity statin | 135 | (82.8%) | 261 | (80.8%) | 396 | (81.5%) |
| Atorvastatin daily dose (mg) | 64 | (39.3%) | 113 | (35.0%) | 177 | (36.4%) |
| 10 | 1 | (0.6%) | 3 | (0.9%) | 4 | (0.8%) |
| 20 | 2 | (1.2%) | 7 | (2.2%) | 9 | (1.9%) |
| 40 | 23 | (14.1%) | 23 | (7.1%) | 46 | (9.5%) |
| 80 | 38 | (23.3%) | 77 | (23.8%) | 115 | (23.7%) |
| Other doses | 0 | | 3 | (0.9%) | 3 | (0.6%) |
| Rosuvastatin daily dose (mg) | 81 | (49.7%) | 172 | (53.3%) | 253 | (52.1%) |
| 5 | 4 | (2.5%) | 7 | (2.2%) | 11 | (2.3%) |
| 10 | 2 | (1.2%) | 5 | (1.5%) | 7 | (1.4%) |
| 20 | 19 | (11.7%) | 44 | (13.6%) | 63 | (13.0%) |
| 40 | 55 | (33.7%) | 116 | (35.9%) | 171 | (35.2%) |
| Other doses | 1 | (0.6%) | 0 | | 1 | (0.2%) |
| Simvastatin daily dose (mg) | 18 | (11.0%) | 38 | (11.8%) | 56 | (11.5%) |
| 10 | 2 | (1.2%) | 2 | (0.6%) | 4 | (0.8%) |
| 20 | 1 | (0.6%) | 5 | (1.5%) | 6 | (1.2%) |
| 40 | 10 | (6.1%) | 25 | (7.7%) | 35 | (7.2%) |
| 80 | 3 | (1.8%) | 6 | (1.9%) | 9 | (1.9%) |
| Other doses | 2 | (1.2%) | 0 | | 2 | (0.4%) |
| Any LMT other than statins^{a} | 107 | (65.6%) | 198 | (61.3%) | 305 | (62.8%) |
| Any LMT other than nutraceuticals | 105 | (64.4%) | 192 | (59.4%) | 297 | (61.1%) |
| Ezetimibe | 97 | (59.5%) | 180 | (55.7%) | 277 | (57.0%) |
| Nutraceuticals | 8 | (4.9%) | 20 | (6.2%) | 28 | (5.8%) |

^{a}in combination with statins or not.
High intensity statin corresponds to atorvastatin 40 to 80 mg daily or rosuvastatin 20 to 40 mg daily.

TABLE 8
Lipid efficacy parameters at baseline - Quantitative
summary in conventional units - Randomized population

| | | Alirocumab 75 | |
|---|---|---|---|
| | Placebo | Q2W/Up150 Q2W | All |
| | (N = 163) | (N = 323) | (N = 486) |
| Calculated LDL-C (mg/dL) | | | |
| Number | 163 | 323 | 486 |
| Mean (SD) | 144.4 (46.8) | 144.8 (51.1) | 144.6 (49.7) |
| Median | 138.0 | 135.0 | 135.5 |
| Q1:Q3 | 112.0:166.0 | 112.0:163.0 | 112.0:165.0 |
| Min:Max | 66:354 | 39:384 | 39:384 |
| Measured LDL-C (mg/dL) | | | |
| Number | 140 | 272 | 412 |
| Mean (SD) | 140.0 (43.5) | 140.2 (49.7) | 140.1 (47.6) |
| Median | 135.0 | 130.5 | 132.0 |
| Q1:Q3 | 111.0:164.0 | 108.0:159.5 | 108.5:161.0 |
| Min:Max | 68:356 | 37:378 | 37:378 |
| Non-HDL-C (mg/dL) | | | |
| Number | 163 | 323 | 486 |
| Mean (SD) | 169.6 (50.6) | 170.3 (54.6) | 170.1 (53.3) |
| Median | 161.0 | 158.0 | 160.0 |
| Q1:Q3 | 132.0:195.0 | 134.0:198.0 | 133.0:196.0 |
| Min:Max | 78:390 | 58:426 | 58:426 |
| Total-C (mg/dL) | | | |
| Number | 163 | 323 | 486 |
| Mean (SD) | 217.6 (50.3) | 221.1 (54.3) | 219.9 (53.0) |
| Median | 210.0 | 212.0 | 211.0 |
| Q1:Q3 | 185.0:240.0 | 184.0:244.0 | 185.0:243.0 |
| Min:Max | 137:445 | 123:482 | 123:482 |
| HDL-C (mg/dL) | | | |
| Number | 163 | 323 | 486 |
| Mean (SD) | 48.0 (14.4) | 50.8 (15.7) | 49.8 (15.3) |
| Median | 45.0 | 47.0 | 46.5 |
| Q1:Q3 | 36.0:56.0 | 39.0:59.0 | 38.0:58.0 |
| Min:Max | 24:116 | 22:115 | 22:116 |
| Fasting TGs (mg/dL) | | | |
| Number | 163 | 323 | 486 |
| Mean (SD) | 126.5 (62.9) | 128.4 (66.7) | 127.8 (65.4) |
| Median | 111.0 | 113.0 | 112.0 |
| Q1:Q3 | 85.0:151.0 | 82.0:153.0 | 83.0:152.0 |
| Min:Max | 45:431 | 35:566 | 35:566 |
| Lipoprotein-(a)(mg/dL) | | | |
| Number | 161 | 317 | 478 |
| Mean (SD) | 47.2 (51.6) | 51.7 (50.2) | 50.2 (50.7) |
| Median | 23.0 | 34.0 | 28.0 |
| Q1:Q3 | 8.0:72.0 | 12.0:82.0 | 11.0:80.0 |
| Min:Max | 2:223 | 2:229 | 2:229 |
| Apo-B (mg/dL) | | | |
| Number | 161 | 317 | 478 |
| Mean (SD) | 113.4 (28.5) | 114.4 (30.8) | 114.1 (30.0) |
| Median | 109.0 | 108.0 | 109.0 |
| Q1:Q3 | 94.0:128.0 | 94.0:130.0 | 94.0:129.0 |
| Min:Max | 64:249 | 45:250 | 45:250 |
| Apo-A1 (mg/dL) | | | |
| Number | 161 | 317 | 478 |
| Mean (SD) | 137.6 (27.2) | 142.8 (27.4) | 141.1 (27.4) |
| Median | 134.0 | 138.0 | 137.0 |
| Q1:Q3 | 121.0:151.0 | 124.0:158.0 | 122.0:155.0 |
| Min:Max | 84:292 | 79:278 | 79:292 |
| Apo-B/Apo-A1 (ratio) | | | |
| Number | 161 | 317 | 478 |
| Mean (SD) | 0.859 (0.292) | 0.830 (0.269) | 0.839 (0.277) |
| Median | 0.810 | 0.780 | 0.800 |
| Q1:Q3 | 0.640:0.990 | 0.650:0.960 | 0.650:0.970 |
| Min:Max | 0.36:2.42 | 0.26:1.84 | 0.26:2.42 |
| Total-C/HDL-C (ratio) | | | |
| Number | 163 | 323 | 486 |
| Mean (SD) | 4.907 (1.838) | 4.707 (1.756) | 4.774 (1.785) |
| Median | 4.658 | 4.321 | 4.444 |
| Q1:Q3 | 3.661:5.658 | 3.537:5.649 | 3.542:5.649 |
| Min:Max | 1.86:13.64 | 1.73:15.14 | 1.73:15.14 |

Note:
Measured LDL-C was assessed via the beta-quantification method.

The collection of measured LDL-C was not planned in the initial protocol and was added in an amendment. Therefore, measured LDL-C values are available for fewer patients compared to calculated LDL-C values.

### Dosage and Duration

Exposure to injections was similar across treatment groups with a mean exposure of 59 weeks.

In the alirocumab group, among the 311 patients who received at least one injection after Week 12, 135 (43.4%) patients received automatic up-titration from 75 mg Q2W to 150 mg Q2W at Week 12 in a blinded manner.

### Efficacy

### Primary Efficacy Endpoint

The ITT analysis includes all calculated LDL-C values collected on-treatment and off-treatment up to Week 52. The primary endpoint (percent change in calculated LDL-C from baseline to Week 24) analysis is provided based on a MMRM model on the ITT population, using LS means estimates at Week 24. Thirty-two (9.9%) patients in the alirocumab group and 14 (8.6%) patients in the placebo group did not have a calculated LDL-C value at Week 24. These missing values were accounted for by the MMRM model.

Results of the primary endpoint analysis are presented in Table 9, in mmol/L and mg/dL.

### Primary Efficacy Analysis

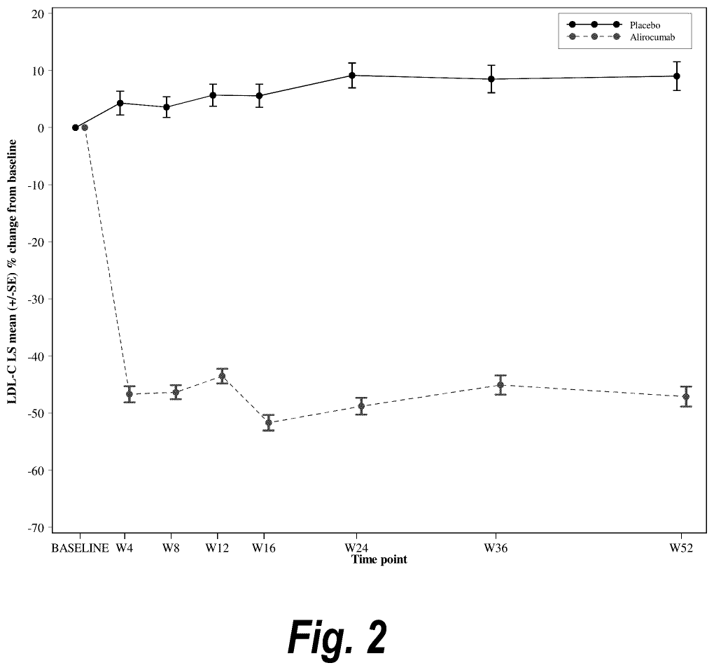

A statistically significant decrease in percent change in LDL-C from baseline to Week 24 was observed in the alirocumab group (LS mean versus baseline—48.8%) compared to the placebo group (LS mean versus baseline+9.1%) (LS mean difference vs. placebo of −57.9%, p<0.0001). In the alirocumab group, LDL-C reduction from baseline was observed from Week 4 and maintained throughout the study up to Week 78 (see FIG. 2 and Table 10).

TABLE 9
Percent change from baseline in calculated LDL-C
at Week 24: MMRM - ITT analysis - ITT population

| | | Alirocumab 75 |
|---|---|---|
| Calculated LDL | Placebo | Q2W/Up150 Q2W |
| Cholesterol | (N = 163) | (N = 322) |
| Baseline (mmol/L) | | |
| Number | 163 | 322 |
| Mean (SD) | 3.739 (1.213) | 3.748 (1.326) |
| Median | 3.574 | 3.497 |
| Min:Max | 1.71:9.17 | 1.01:9.95 |
| Baseline (mg/dL) | | |
| Number | 163 | 322 |
| Mean (SD) | 144.4 (46.8) | 144.7 (51.2) |
| Median | 138.0 | 135.0 |
| Min:Max | 66:354 | 39:384 |
| Week 24 percent change | | |
| from baseline (%) | | |
| LS Mean (SE) | 9.1 (2.2) | −48.8 (1.6) |
| LS mean difference (SE) | | −57.9 (2.7) |
| vs placebo | | |
| 95% CI | | (−63.3 to −52.6) |
| p-value vs placebo | | <0.0001* |

Note:
Least-squares (LS) means, standard errors (SE) and p-value taken from MMRM (mixed-effect model with repeated measures) analysis. The model includes the fixed categorical effects of treatment group, randomization strata as per IVRS, time point, treatment-by-time point and strata-by-time point interaction, as well as the continuous fixed covariates of baseline calculated LDL-C value and baseline calculated LDL-C value-by-time point interaction
MMRM model and baseline description run on patients with a baseline value and a post-baseline value in at least one of the analysis windows used in the model.
The p-value is followed by a ‘*’ if statistically significant according to the fixed hierarchical approach used to ensure a strong control of the overall type-I error rate at the 0.05 level

TABLE 10
Calculated LDL-C over time - ITT analysis - ITT population

| | | | | | | Alirocumab 75 | | | | |
|---|---|---|---|---|---|---|---|---|---|---|
| | Placebo | | | | | Q2W/Up150 Q2W | | | | |
| | (N = 163) | | | | | (N = 322) | | | | |
| | | | | | Percent | | | | | Percent |
| | | | Change | | change | | | Change | | change |
| Calculated | | | from | | from | | | from | | from |
| LDL-C | Value | | baseline | | baseline | Value | | baseline | | baseline |
| LS Mean (SE) (mmol/L) | | | | | | | | | | |
| Baseline ^{a} | 3.739 | (0.095) | NA | | NA | 3.748 | (0.074) | NA | | NA |
| Week 4 | 3.819 | (0.070) | 0.074 | (0.070) | 4.3 (2.1) | 1.996 | (0.050) | −1.749 | (0.050) | −46.7 (1.5) |
| Week 8 | 3.805 | (0.073) | 0.059 | (0.073) | 3.6 (1.8) | 1.986 | (0.052) | −1.759 | (0.052) | −46.4 (1.3) |
| Week 12 | 3.898 | (0.074) | 0.153 | (0.074) | 5.7 (2.0) | 2.078 | (0.053) | −1.668 | (0.053) | −43.5 (1.4) |
| Week 16 | 3.892 | (0.080) | 0.147 | (0.080) | 5.6 (2.1) | 1.763 | (0.057) | −1.982 | (0.057) | −51.7 (1.5) |
| Week 24 | 4.029 | (0.084) | 0.284 | (0.084) | 9.1 (2.2) | 1.846 | (0.060) | −1.899 | (0.060) | −48.8 (1.6) |
| Week 36 | 3.965 | (0.091) | 0.220 | (0.091) | 8.5 (2.4) | 1.997 | (0.066) | −1.748 | (0.066) | −45.1 (1.8) |
| Week 52 | 4.000 | (0.092) | 0.255 | (0.092) | 9.0 (2.6) | 1.925 | (0.066) | −1.821 | (0.066) | −47.1 (1.9) |
| Week 64 | 3.947 | (0.086) | | | | 1.962 | (0.063) | | | |
| Week 78 | 4.082 | (0.101) | | | | 2.177 | (0.073) | | | |
| LS Mean (SE) (mg/dL) | | | | | | | | | | |
| Baseline ^{a} | 144.4 | (3.7) | NA | | NA | 144.7 | (2.9) | NA | | NA |
| Week 4 | 147.5 | (2.7) | 2.9 | (2.7) | 4.3 (2.1) | 77.1 | (1.9) | −67.5 | (1.9) | −46.7 (1.5) |
| Week 8 | 146.9 | (2.8) | 2.3 | (2.8) | 3.6 (1.8) | 76.7 | (2.0) | −67.9 | (2.0) | −46.4 (1.3) |
| Week 12 | 150.5 | (2.9) | 5.9 | (2.9) | 5.7 (2.0) | 80.2 | (2.0) | −64.4 | (2.0) | −43.5 (1.4) |
| Week 16 | 150.3 | (3.1) | 5.7 | (3.1) | 5.6 (2.1) | 68.1 | (2.2) | −76.5 | (2.2) | −51.7 (1.5) |
| Week 24 | 155.6 | (3.2) | 11.0 | (3.2) | 9.1 (2.2) | 71.3 | (2.3) | −73.3 | (2.3) | −48.8 (1.6) |
| Week 36 | 153.1 | (3.5) | 8.5 | (3.5) | 8.5 (2.4) | 77.1 | (2.5) | −67.5 | (2.5) | −45.1 (1.8) |
| Week 52 | 154.4 | (3.5) | 9.8 | (3.5) | 9.0 (2.6) | 74.3 | (2.6) | −70.3 | (2.6) | −47.1 (1.9) |
| Week 64 | 152.4 | (3.3) | | | | 75.8 | (2.4) | | | |
| Week 78 | 157.6 | (3.9) | | | | 84.0 | (2.8) | | | |

^{a} Baseline is described using means and standard errors.
Note:
Least-squares (LS) means, standard errors (SE) and p-value taken from MMRM (mixed-effect model with repeated measures) analysis. The model includes the fixed categorical effects of treatment group, randomization strata as per IVRS, time point, treatment-by-time point interaction, strata-by-time point interaction, as well as the continuous fixed covariates of baseline LDL-C value and baseline LDL-C value-by-time point interaction
MMRM model and baseline description run on patients with a baseline value and a post-baseline value in at least one of the analysis windows used in the model.

### Key Secondary Efficacy Endpoints

Table 11 summarizes analysis results on key secondary endpoints in the hierarchical order. All key secondary endpoints are statistically significant according to the hierarchical testing procedure.

TABLE 11

| Endpoint | Analysis | Results | P-value |
|---|---|---|---|
| Calculated LDL-C - Percent change | On-treatment | LS mean difference vs. | <0.0001 |
| from baseline to Week 24 | | placebo of −58.1% | |
| Calculated LDL-C - Percent change | ITT | LS mean difference vs. | <0.0001 |
| from baseline to Week 12 | | placebo of −49.2% | |
| Calculated LDL-C - Percent change | On-treatment | LS mean difference vs. | <0.0001 |
| from baseline to Week 12 | | placebo of −49.5% | |
| Apo-B - Percent change from | ITT | LS mean difference vs. | <0.0001 |
| baseline to Week 24 | | placebo of −45.8% | |
| Apo-B - Percent change from | On-treatment | LS mean difference vs. | <0.0001 |
| baseline to Week 24 | | placebo of −45.9% | |
| Non-HDL-C - Percent change from | ITT | LS mean difference vs. | <0.0001 |
| baseline to Week 24 | | placebo of −52.4% | |
| Non-HDL-C - Percent change from | On-treatment | LS mean difference vs. | <0.0001 |
| baseline to Week 24 | | placebo of −52.6% | |
| Total-C - Percent change from | ITT | LS mean difference vs. | <0.0001 |
| baseline to Week 24 | | placebo of −38.7% | |
| Apo-B - Percent change from | ITT | LS mean difference vs. | <0.0001 |
| baseline to Week 12 | | placebo of −37.5% | |
| Non-HDL-C - Percent change from | ITT | LS mean difference vs. | <0.0001 |
| baseline to Week 12 | | placebo of −43.7% | |
| Total-C - Percent change from | ITT | LS mean difference vs. | <0.0001 |
| baseline to Week 12 | | placebo of −32.5% | |
| Calculated LDL-C - Percent change | ITT | LS mean difference vs. | <0.0001 |
| from baseline to Week 52 | | placebo of −56.2% | |
| Proportion of very high CV risk | ITT | combined estimate for odds- | <0.0001 |
| patients reaching calculated LDL- | | ratio vs. placebo of 155.1 | |
| C <70 mg/dL (1.81 mmol/L) or high | | | |
| CV risk patients reaching | | | |
| calculated LDL-C <100 mg/dL | | | |
| (2.59 mmol/L) at Week 24 | | | |
| Proportion of very high CV risk | On-treatment | combined estimate for odds- | <0.0001 |
| patients reaching calculated LDL- | | ratio vs. placebo of 149.1 | |
| C <70 mg/dL (1.81 mmol/L) or high | | | |
| CV risk patients reaching | | | |
| calculated LDL-C <100 mg/dL | | | |
| (2.59 mmol/L) at Week 24 | | | |
| Proportion of patients reaching | ITT | combined estimate for odds- | <0.0001 |
| calculated LDL-C <70 mg/dL (1.81 | | ratio vs. placebo of 237.1 | |
| mmol/L) at Week 24 | | | |
| Proportion of patients reaching | On-treatment | combined estimate for odds- | <0.0001 |
| calculated LDL-C <70 mg/dL (1.81 | | ratio vs. placebo of 237.9 | |
| mmol/L) at Week 24 | | | |
| Lp(a) - Percent change from | ITT | combined estimate for | <0.0001 |
| baseline to Week 24 | | adjusted mean difference | |
| | | vs. placebo of −17.7% | |
| HDL-C - Percent change from | ITT | LS mean difference vs. | <0.0001 |
| baseline to Week 24 | | placebo of 8% | |
| Fasting TGs - Percent change from | ITT | combined estimate for | <0.0001 |
| baseline to Week 24 | | adjusted mean difference | |
| | | vs. placebo of −16.1% | |
| Apolipoprotein A1 - Percent change | ITT | LS mean difference vs. | <0.05 |
| from baseline to Week 24 | | placebo of 4.7% | |

The on-treatment analysis of LDL-C percent change from baseline to Week 24 shows very consistent results with the ITT analysis (LS mean difference vs. placebo of −58.1% in the on-treatment analysis versus—57.9% in the ITT analysis). Indeed, few patients had LDL-C values collected post-treatment (i.e., more than 21 days after last injection) at Week 24: 6 patients (3.7%) in the placebo group and 2 patients (0.6%) in the alirocumab group. A statistically significant decrease in percent change in LDL-C from baseline to Week 12 (i.e. before possible up-titration) in the ITT analysis was observed in the alirocumab group (LS mean versus baseline—43.5%) compared to the placebo group (LS mean versus baseline+5.7%) (LS mean difference vs. placebo of −49.2%, p<0.0001).

The key secondary endpoints of Apo B, non-HDL-C, Total-C, Lp(a), HDL-C, and TGs at various time points as well as the proportion of patients reaching their LDL-C goals and the proportion of patients reaching calculated LDLD-C<70 mg/dL at Week 24 were statistically significant according to the hierarchical testing procedure. For the alirocumab group the baseline mean (SD) LDL-C, Non-LDL-C, ApoB and the median (IQR) Lp(a) levels were 144.7 (51.3), 170.3 (54.6), 114.3 (30.8), and 34 (12:82) mg/dl respecticely. For the placebo group the baseline mean (SD) LDL-C, Non-LDL-C, ApoB and the median (IQR) Lp(a) levels were 144.4 (46.8), 169.6 (50.6), 113.4 (28:5), and 23 (8.72) mg/dl respectively. After 24 weeks, LS mean (SE) % change from baseline to Week 24 for Non-LDL-C, ApoB Lp(a) levels in the alirocumab group was −42.8%, −41.1%, and −25.2%, respectively. The LS mean (SE) % change from baseline to Week 24 for Non-LDL-C, ApoB Lp(a) levels for the placebo group was 9.6%, 4.7%, and −7.5%, respectively. The LS mean difference vs. placebo for Non-LDL-C, ApoB and Lp(a) was −52.4%, −45.8%, and 17.7%, respectively.

The proportion of very high cardiovascular (CV) risk patients reaching calculated LDL-C<70 mg/dL (1.81 mmol/L) or high CV risk patients reaching calculated LDL-C<100 mg/dL (2.59 mmol/L) at Week 24 was significantly higher in the alirocumab than in the placebo group (combined estimate for proportion of 72.1% in the alirocumab group vs 2.4% in the placebo group, p<0.0001).

Two consecutive calculated LDL-C values <25 mg/dL (<0.65 mmol/L) were observed in 16 (5.0%) patients. No particular safety concern has been observed in these patients.

TABLE 12
Number (%) of patients with 2 consecutive calculated LDL-C <25 mg/dL
(<0.65 mmol/L) during the treatment period- Safety population

| | |
|---|---|
| | Alirocumab 75 |
| Placebo | Q2W/Up150 Q2W |
| (N = 163) | (N = 322) |

Patients with 2 consecutive calculated LDL-C value <25 mg/dL ¹

| | |
|---|---|
| 0/163 | 16/317 (5.0%) |

Time to the first calculated LDL-C value <25 mg/dL (weeks) ²

| | | |
|---|---|---|
| Number | 0 | 16 |
| Mean (SD) | | 14.79 (11.37) |
| Median | | 14.14 |
| Min:Max | | 3.1:36.1 |

Patients with 2 consecutive calculated LDL-C value <15 mg/dL ¹

| | |
|---|---|
| 0/163 | 6/317 (1.9%) |

Time to the first calculated LDL-C value <15 mg/dL (weeks) ²

| | | |
|---|---|---|
| Number | 0 | 6 |
| Mean (SD) | | 18.31 (12.35) |
| Median | | 20.14 |
| Min:Max | | 4.6:36.1 |

The number (n) represents the subset of the total number of patients who met the criteria
The denominator (/N) within a treatment group is the number of patients for the treatment group who had at least two calculated LDL-C values assessed at least 21 days apart in the efficacy period
¹ 2 consecutive values are considered if spaced out by at least 21 days
² First calculated LDL-C value <25 or <15 mg/dL among the first 2 consecutive calculated LDL-C values <25 or <15 mg/dL per patient

### Summary Safety Results:

Alirocumab was well tolerated during the treatment period.

TABLE 13
Overview of adverse event profile: Treatment
emergent adverse events - Safety population

| | | | Alirocumab 75 | |
|---|---|---|---|---|
| | Placebo | | Q2W/Up150 Q2W | |
| n(%) | (N = 163) | | (N = 322) | |
| Patients with any TEAE | 122 | (74.8%) | 249 | (77.3%) |
| Patients with any treatment | 15 | (9.2%) | 39 | (12.1%) |
| emergent SAE | | | | |
| Patients with any TEAE | 0 | | 4 | (1.2%) |
| leading to death | | | | |
| Patients with any TEAE | 8 | (4.9%) | 10 | (3.1%) |
| leading to permanent treatment | | | | |
| discontinuation | | | | |

n (%) = number and percentage of patients with at least one TEAE

Overall, the proportions of patients reporting at least one treatment emergent adverse event (TEAE) (77.3% in the alirocumab group and 74.8% in the placebo group) or at least one TEAE leading to permanent discontinuation (3.1% in the alirocumab group and 4.9% in the placebo group) were similar in both groups. “Musculoskeletal and connective tissue disorders” SOC was reported in 22.4% of patients in the alirocumab group vs. 25.2% in the placebo group. The most frequently reported TEAEs in both treatment groups were “injection site reaction” (11.8% vs. 9.8% in alirocumab vs. placebo group, respectively) and “nasopharyngitis” (9.9% vs 6.7% in alirocumab vs. placebo group, respectively). Among the events of interest, no particular signal was detected for TEAEs related to allergic events, neurological events, neurocognitive disorders and diabetes. The SOC “neoplasms begnin, malignant and unspecified” was observed in 2.8% of patients in the alirocumab group vs 0.6% in the placebo group with no particular clinical pattern on individual events (all these events were reported as not related to IMP by the investigator). TEAEs “cardiovascular events confirmed by adjudication” were reported for 1.9% of patients in the alirocumab group and 1.2% in the placebo group.

Six deaths (1.9%) were reported as not related to IMP by the investigator in the alirocumab group versus none in the placebo group: two myocardial infarctions (MI) (one classified as acute MI and one classified as sudden cardiac death), two metastatic cancers (non-small cell lung cancer and pancreatic carcinoma with secondary Trousseau syndrome causing multiple embolic strokes), a colonic pseudo-obstruction following abdominal surgery in one patient, and sudden cardiac death in one patient due to congestive cardiac failure and coronary artery disease. Both patients with MI had multiple risk factors for coronary artery disease. With regards to cancers, the time to onset of first symptoms (about 3.5 and 7.5 months after starting the investigational product) is not suggestive of a causal role of the investigational product.

No relevant abnormalities were observed for PCSA.

### Example 3: A Randomized, Double-Blind, Placebo-Controlled, Parallel-Group Study to Evaluate the Efficacy and Safety of Alirocumab in Patients with Heterozygous Familial Hypercholesterolemia not Adequately Controlled with their Lipid-Modifying Therapy

### Introduction

The objective of the study was to assess the efficacy and safety of Alirocumab in improving lipid parameters in patients with heterozygous familial hypercholesterolemia (heFH) who have failed to reach their LDL-C treatment goal on maximally-tolerated statin therapy, with or without additional lipid-modifying therapy (LMT). Patients not at goal on a maximally-tolerated dose of daily statin therapy, with or without other LMT, were enrolled in this study, and that their background treatment was maintained throughout the study.

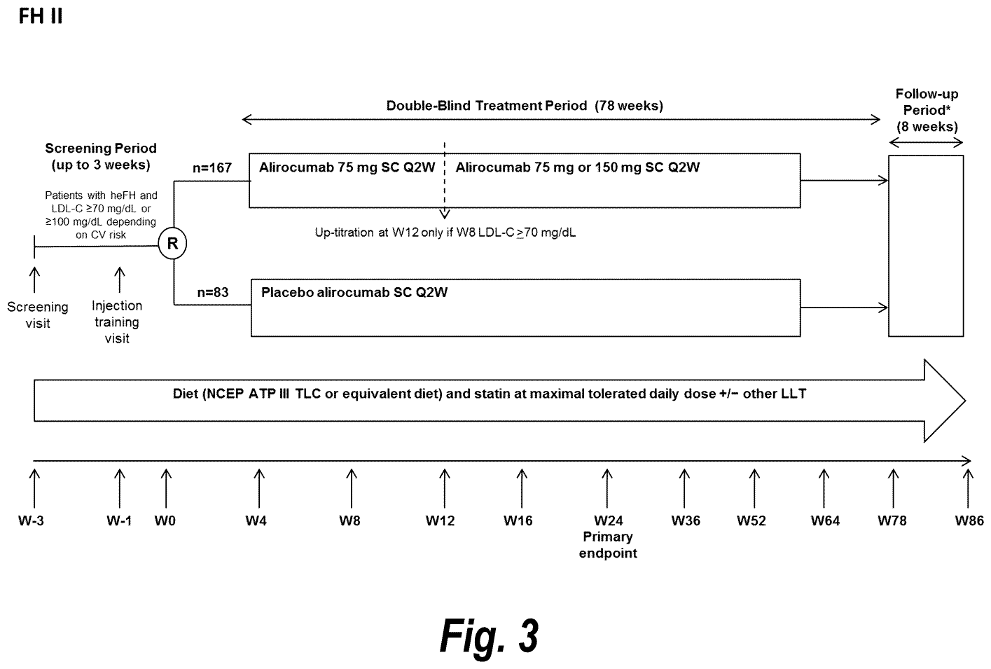

This specific study (FIG. 3) was undertaken to demonstrate in heFH patients who were not at their LDL-C goal, that Alirocumab 75 mg q2w or 75 mg q2w/150 mg q2w as add-on therapy to statin+/−other LMT, causes a statistically significant and clinically meaningful reduction in LDL-C. This population that is not at LDL-C goal on optimized LMT represents a highest risk group with a well-identified unmet medical need that can be addressed by adding Alirocumab to their LDL-C lowering therapies.

### Study Objectives

The primary objective of the study was to demonstrate the reduction of LDL-C by Alirocumab as add-on therapy to stable, maximally-tolerated daily statin therapy with or without other LMT in comparison with placebo after 24 weeks of treatment in patients with heFH.

The secondary objectives of the study were: 1) to evaluate the effect of Alirocumab 75 mg in comparison with placebo on LDL-C after 12 weeks of treatment; 2) to evaluate the effect of Alirocumab on other lipid parameters (e.g., ApoB, non-HDL-C, total-C, Lp[a], HDL-C, TG levels, and ApoA-1 levels); 3) to evaluate the long-term effect of Alirocumab on LDL-C; 4) to evaluate the safety and tolerability of Alirocumab; and 5) to evaluate the development of anti-Alirocumab antibodies.

### Study Design

This was a randomized, double-blind, placebo-controlled, parallel-group, multi-national study in patients with heFH who were not adequately controlled with their LMT (i.e., stable maximally-tolerated daily statin therapy+/−other LMT). Not adequately controlled was defined as an LDL-C≥70 mg/dL (1.81 mmol/L) at the screening visit (week 2) in patients with a history of documented CVD or LDL-C≥100 mg/dL (2.59 mmol/L) at the screening visit (week −2) in patients without a history of documented CVD. Patients were randomized in a 2:1 ratio to receive either 75 mg of Alirocumab or placebo by SC injection, every 2 weeks, on top of stable, maximally-tolerated daily statin therapy (atorvastatin, rosuvastatin, or simvastatin) with or without other LMT. Randomization was stratified according to prior history of either myocardial infarction (MI) or ischemic stroke, and statin treatment (atorvastatin 40 mg to 80 mg daily or rosuvastatin 20 mg to 40 mg daily vs. simvastatin whatever the daily dose, atorvastatin below 40 mg daily, or rosuvastatin below 20 mg daily).

The study consisted of three periods: a screening period, a treatment period, and a follow-up period.

The screening period was up to 2 weeks, including an intermediate visit during which the patient or caregiver was trained to self-inject/inject using a dose of placebo.

The double-blind treatment period was 78 weeks. The first injection of study drug was administered at the clinical site on day 1, after study assessments were completed, and as soon as possible after the patient was randomized into the study. The patient/caregiver administered subsequent injections outside of the clinic according to the dosing schedule. On days where the clinic study visit coincides with dosing, the dose of study drug was administered after all study assessments were performed and all laboratory samples collected. The last dose of study drug was administered at week 76. At week 12, patients randomized to Alirocumab were, in a blinded manner, either: 1) continued Alirocumab 75 mg every 2 weeks, if the week 8 LDL-C was <70 mg/dL (1.81 mmol/L), or 2) dose up-titrated to Alirocumab 150 mg every 2 weeks, if the week 8 LDL-C was ≥70 mg/dL (1.81 mmol/L).

The follow-up period (if applicable) was 8 weeks after the end of the DBTP for patients not consenting to participate in the open-label extension study, or if prematurely discontinuing study treatment.

Patients were asked to follow a stable diet (equivalent to the National Cholesterol Education Program Adult Treatment Panel III Therapeutic Lifestyle Changes [NCEP ATP III TLC] diet/Appendix 5) from screening to the end of study visit. The daily dose of statin or other LMT (if applicable) should remain stable from screening to the end of study visit. Starting at week 24, background LMT may be modified under certain conditions as described later. Table 1 from Example 2 is relevant to this Example and provides a summary of the TLC diet for high cholesterol.

An independent external physician was notified by the central laboratory for any patient who achieved 2 consecutive calculated LDL-C levels <25 mg/dL (0.65 mmol/L). Patients who meet this criterion were monitored.

### Selection of Patients

The study population consisted of patients with heFH who were not adequately controlled with a maximally-tolerated stable daily dose of a statin for at least 4 weeks before the screening visit (week −2), with or without other LMT.

A patient must have met the following criteria to be eligible for inclusion in the study: 1) patients with heFH* who were not adequately controlled** with a maximally-tolerated daily dose*** of statin with or without other LMT, at a stable dose prior to the screening visit (week −2). *Diagnosis of heFH must be made either by genotyping or by clinical criteria. For those patients not genotyped, the clinical diagnosis may be based on either the Simon Broome criteria for definite FH or the WHO/Dutch Lipid Network criteria with a score of >8 points.**“Not adequately controlled” was defined herein the same as it was in Example 2.***“Maximally-tolerated dose” was defined herein the same as it was in Example 2.

Definite familial hypercholesterolemia was defined herein the same as it was in Example 2. Possible familial hypercholesterolemia was defined herein the same as it was in Example 2. The WHO Criteria (Dutch Lipid Network clinical criteria) for Diagnosis of Heterozygous Familial Hypercholesterolemia (heFH) set forth in Table 2 in Example 2 was the same for this Example.

A Documented History of CHD was defined herein the same as in Example 2.

CHD Risk Equivalents (includes 1 or more of the following criteria): 1) documented peripheral arterial disease (one of the following criteria must be satisfied): A) current intermittent claudication (muscle discomfort in the lower limb produced by exercise that is both reproducible and relieved by rest within 10 minutes) of presumed atherosclerotic origin together with ankle-brachial index equal to or less than 0.90 in either leg at rest, or B) history of intermittent claudication (muscle discomfort in the lower limb produced by exercise that is both reproducible and relieved by rest within 10 minutes) together with endovascular procedure or surgical intervention in one or both legs because of atherosclerotic disease, or C) history of critical limb ischemia together with thrombolysis, endovascular procedure or surgical intervention in one or both legs because of atherosclerotic disease; 2) documented previous ischemic stroke with a focal ischemic neurological deficit that persisted more than 24 hours, considered as being of atherothrombotic origin. CT or MRI must have been performed to rule out hemorrhage and non-ischemic neurological disease.

Patients who met all of the above inclusion criteria were screened for the following exclusion criteria, which are sorted in the following three subsections: exclusion criteria related to study methodology, exclusion criteria related to the active comparator and/or mandatory background therapies, and exclusion criteria related to the current knowledge of Alirocumab.

Exclusion criteria related to the study methodology were: 1) patient without diagnosis of heFH made either by genotyping or by clinical criteria; 2) LDL-C<70 mg/dL (<1.81 mmol/L) at the screening visit (week −2) in patients with history of documented CVD. NOTE: CVD is defined as CHD, ischemic stroke, or peripheral arterial disease as described above; 3) LDL-C<100 mg/dL (<2.59 mmol/L) at the screening visit (week −2) in patients without history of documented CVD; 4) not on a stable dose of LMT (including statin) for at least 4 weeks and/or fenofibrate for at least 6 weeks, as applicable, prior to the screening visit (week −2) and from screening to randomization; 5) currently taking another statin than simvastatin, atorvastatin, or rosuvastatin; 6) simvastatin, atorvastatin, or rosuvastatin is not taken daily or not taken at a registered dose; 7) daily doses above atorvastatin 80 mg, rosuvastatin 40 mg, or simvastatin 40 mg (except for patients on simvastatin 80 mg for more than 1 year, who are eligible); 8) use of fibrates, other than fenofibrate within 6 weeks of the screening visit (week −2) or between screening and randomization visits; 9) use of nutraceutical products or over-the-counter therapies that may affect lipids which have not been at a stable dose/amount for at least 4 weeks prior to the screening visit (week −2) or between screening and randomization visits; 10) use of red yeast rice products within 4 weeks of the screening visit (week −2), or between screening and randomization visits; 11) patient who has received plasmapheresis treatment within 2 months prior to the screening visit (week −2), or has plans to receive it during the study; 12) recent (within 3 months prior to the screening visit [week −2] or between screening and randomization visits) MI, unstable angina leading to hospitalization, percutaneous coronary intervention (PCI), coronary artery bypass graft surgery (CABG), uncontrolled cardiac arrhythmia, stroke, transient ischemic attack, carotid revascularization, endovascular procedure or surgical intervention for peripheral vascular disease; 13) planned to undergo scheduled PCI, CABG, carotid, or peripheral revascularization during the study; 14) systolic blood pressure >160 mm Hg or diastolic blood pressure >100 mm Hg at screening visit or randomization visit; 15) history of New York Heart Association (NYHA) Class III or IV heart failure within the past 12 months; 16) known history of a hemorrhagic stroke; 17) age <18 years or legal age of majority at the screening visit (week −2), whichever is greater; 18) patients not previously instructed on a cholesterol-lowering diet prior to the screening visit (week −2); 19) newly diagnosed (within 3 calendar months prior to randomization visit [week 0]) or poorly controlled (hemoglobin A1c [HbA1c]>9% at the screening visit [week −2]) diabetes; 20) presence of any clinically significant uncontrolled endocrine disease known to influence serum lipids or lipoproteins. Note: Patients on thyroid replacement therapy can be included if the dosage has been stable for at least 12 weeks prior to screening and between screening and randomization visits, and thyroid-stimulating hormone (TSH) level is within the normal range of the central laboratory at the screening visit; 21) history of bariatric surgery within 12 months prior to the screening visit (week −2); 22) unstable weight defined by a variation >5 kg within 2 months prior to the screening visit (week −2); 23) known history of homozygous FH; 24) known history of loss-of-function of PCSK9 (ie, genetic mutation or sequence variation); 25) use of systemic corticosteroids, unless used as replacement therapy for pituitary/adrenal disease with a stable regimen for at least 6 weeks prior to randomization visit (week 0). Note: Topical, intra-articular, nasal, inhaled and ophthalmic steroid therapies are not considered as ‘systemic’ and are allowed; 26) use of continuous estrogen or testosterone hormone replacement therapy unless the regimen has been stable in the past 6 weeks prior to the screening visit (week −2) and no plans to change the regimen during the study; 27) history of cancer within the past 5 years, except for adequately treated basal cell skin cancer, squamous cell skin cancer, or in situ cervical cancer; 28) known history of a positive HIV test; 29) patient who has taken any investigational drugs other than the Alirocumab training placebo kits within 1 month or 5 half-lives, whichever is longer; 30) patient who has been previously treated with at least 1 dose of Alirocumab or any other anti-PCSK9 monoclonal antibody in other clinical studies; 31) conditions/situations such as: a) any clinically significant abnormality identified at the time of screening that, in the judgment of the investigator or any sub-investigator, would preclude safe completion of the study or constrain endpoints assessment; eg, major systemic diseases, patients with short life expectancy; or b) considered by the investigator or any sub-investigator as inappropriate for this study for any reason, e.g.: i) deemed unable to meet specific protocol requirements, such as scheduled visits; ii) those deemed unable to administer or tolerate long-term injections as per the patient or the investigator; iii) investigator or any sub-investigator, pharmacist, study coordinator, other study staff or relative thereof directly involved in the conduct of the protocol, etc.; iv) presence of any other conditions (eg, geographic or social), either actual or anticipated, that the investigator feels would; 32) laboratory findings during screening period (not including randomization week 0 labs, unless otherwise noted): i) positive test for hepatitis B surface antigen or hepatitis C antibody (confirmed by reflexive testing); ii) positive serum beta-hCG or urine pregnancy test (including week 0) in women of childbearing potential; iii) TG>400 mg/dL (>4.52 mmol/L) (1 repeat lab is allowed); iv) eGFR<30 mL/min/1.73 m2 according to 4-variable MDRD study equation (calculated by central lab); v) alanine aminotransferase (ALT) or aspartate aminotransferase (AST) >3×upper limit of normal (ULN) (1 repeat lab is allowed); vi) CPK>3×ULN (1 repeat lab is allowed); vii) TSH<lower limit of normal (LLN) or >ULN (1 repeat lab is allowed).

Exclusion criteria related to the active comparator and/or mandatory background therapies were: 1) all contraindications to the background therapies or warnings/precautions of use (when appropriate) as displayed in the respective National Product Labeling.

Exclusion criteria related to the current knowledge of Alirocumab were: 1) known hypersensitivity to monoclonal antibody or any component of the drug product; 2) pregnant or breast-feeding women; 3) women of childbearing potential who are not protected by highly-effective method(s) of birth control (as defined in the informed consent form and/or in a local protocol addendum) and/or who are unwilling or unable to be tested for pregnancy. Note: Women of childbearing potential must have a confirmed negative pregnancy test at screening and randomization visits. They must use an effective contraceptive method throughout the entire duration of study treatment and for 10 weeks after the last dose of study drug, and agree to repeat urine pregnancy test at designated visits. The applied methods of contraception have to meet the criteria for a highly effective method of birth control according to the “Note for guidance on non-clinical safety studies for the conduct of human clinical trials for pharmaceuticals (CPMP/ICH/286/95)”. Postmenopausal women must be amenorrheic for at least 12 months.

### Study Treatments

The study treatment was a single SC injection of 1 mL for a 75 mg or 150 mg dose of Alirocumab or placebo provided in an auto-injector, administered in the abdomen, thigh, or outer area of the upper arm. The first injection of study drug was administered at the clinical site, as soon as possible after the patient was randomized into the study. The patient was monitored at the clinical site for 30 minutes following the first injection. The patient/caregiver administered subsequent injections outside of the clinic, according to the dosing schedule. On days where the clinic study visit coincided with dosing, the dose of study drug was administered after all study assessments were performed and all laboratory samples collected. Subcutaneous dosing of study drug should be administered every 2 weeks at approximately the same time of day (based upon patient preference); it was acceptable for dosing to fall within a window of +/−3 days.

Sterile Alirocumab drug product was supplied at a concentration of 75 mg/mL or 150 mg/mL in histidine, pH 6.0, polysorbate 20, and sucrose in an auto-injector.

Placebo matching Alirocumab was supplied in the same formulation as Alirocumab, without the addition of protein, in an auto-injector.

All patients were on a maximally-tolerated stable daily statin (atorvastatin, rosuvastatin, or simvastatin)+/−other LMT throughout the duration of the study. Statin dose and the dose of other LMT (if applicable) should have remained stable throughout the whole study duration, from screening to the end of study visit.

During the double-blind treatment period, modification to the background LMT was allowed before week 24 only under certain conditions: 1) exceptional circumstances—overriding concerns (including, but not limited to, TG alert, below, posted by the central lab) warrant such changes, per the investigator's judgment; or 2) a confirmed TG alert—the patient meets the pre-specified TG alert (TG≥500 mg/dL [5.65 mmol/L]).

During the double-blind treatment period, modification to the background LMT was allowed after week 24 only under certain conditions: 1) exceptional circumstances, per the investigator's judgment; 2) a confirmed TG alert—the patient meets the pre-specified TG alert (TG≥500 mg/dL [5.65 mmol/L], or 3) LDL-C increased by at least 25% as compared to the randomization visit LDL-C (and no other reasonable explanation exists).

For a laboratory rescue alert of LDL-C increase >25% as compared to the randomization visit LDL-C on 2 consecutive occasions, the investigator should have ensured that no reasonable explanation exists for insufficient LDL-C control (such as an alternative medical cause like corticosteroid use, etc.) and in particular that: compliance with diet was appropriate; compliance with background LMT was appropriate; and study treatment was given as planned. If any of the above could reasonably explain the insufficient LDL-C control, the investigator should have stressed the absolute need to be compliant with treatment and, if needed, organized a specific interview with a qualified nutrition professional and stressed the absolute need to be compliant with diet, and performed a blinded LDL-C assessment within 1 to 2 months. Rescue treatment may be initiated in the event that no reason for LDL-C above the threshold value could be found.

If no reason for LDL-C above the threshold value could be found, or if appropriate action failed to decrease LDL-C below the threshold value, rescue medication may have been introduced. The effectiveness of any such changes would be made based on lack of rescue threshold from blinded lipid testing at the next routinely scheduled lab draw. Patients per protocol already received a maximum tolerated dose of statin, so statin up-titration or switch would not be an option. For further LDL-C lowering, the investigator may have considered adding: a cholesterol absorption inhibitor (ezetimibe), or a bile acid-binding sequestrant (the resins cholestyramine and colestipol, or colesevelam, a nonabsorbable polymer). The following lipid modifying agents may have also been considered: fibrate (Note: Caution should be exercised when combining fibrates with other cholesterol-lowering medications such as statins because of the risk of myopathy. When a fibrate is combined with a statin, fenofibrate is the fibrate of choice because it does not affect statin glucuronidation. The only fibrate allowed per protocol was fenofibrate); nicotinic acid (niacin) (Note: Niacin raises blood glucose but has been shown to be effective in modifying lipid disorders in people with diabetes if glucose control is maintained).

The dose of study drug was increased (up-titrated) from 75 mg to 150 mg SC every 2 weeks, starting at week 12, for an individual patient in the event LDL-C≥70 mg/dL at the week 8 visit.

Patients were randomized to receive either Alirocumab or placebo in a ratio of 2:1, with permuted-block randomization. Randomization was stratified according to prior history of MI or ischemic stroke (Yes/No), and statin dose (“Yes” as atorvastatin 40 mg to 80 mg daily or rosuvastatin 20 mg to 40 mg daily and “No” as simvastatin whatever the daily dose, atorvastatin below 40 mg daily or rosuvastatin below 20 mg daily) as fixed effects; and the baseline calculated LDL-C as covariate.

Concomitant medications should have been kept to a minimum during the study. If considered necessary for the patient's welfare and unlikely to interfere with study drug, concomitant medications (other than those that are prohibited during the study) could have been given at the discretion of the investigator, with a stable dose (when possible).

Nutraceutical products or over-the-counter therapies that may affect lipids were allowed only if they had been used at a stable dose for at least 4 weeks before the screening visit, during the screening period, and maintained during the first 24 weeks of the double-blind treatment period. After the week 24 visit, modification to these nutraceutical products or over-the-counter therapies was allowed, but in general should have been avoided. Examples of such nutraceutical products or over-the-counter therapies include omega-3 fatty acids at doses <1000 mg, plant stanols such as found in Benecol, flax seed oil, and psyllium.

Women of childbearing potential must have used an effective contraception method throughout study treatment, and for 10 weeks after the last dose of study drug.

Prohibited concomitant medications from the initial screening visit until the end of the study visit included the following: statins, other than atorvastatin, rosuvastatin, or simvastatin; fibrates, other than fenofibrate; and red yeast rice products.

### Study Endpoints

Baseline characteristics included standard demography (e.g., age, race, weight, height, etc.), disease characteristics including medical history, and medication history for each patient.

The primary efficacy endpoint was the percent change in calculated LDL-C from baseline to week 24, which was defined as: 100× (calculated LDL-C value at week 24−calculated LDL-C value at baseline)/calculated LDL-C value at baseline. The baseline calculated LDL-C value was the last LDL-C level obtained before the first dose of study drug. The calculated LDL-C at week 24 was the LDL-C level obtained within the week 24 analysis window and during the main efficacy period. The main efficacy period was defined as the time from the first double-blind study drug injection up to 21 days after the last double-blind study drug injection or up to the upper limit of the week 24 analysis window, whichever came first.

The key secondary efficacy endpoints were: 1) the percent change in calculated LDL-C from baseline to week 12: similar definition and rules as for primary efficacy endpoint, except that the calculated LDL-C at week 12 was the LDL-C level obtained within the week 12 analysis window and during the 12-week efficacy period. The 12-week efficacy period was defined as the time from the first double-blind study drug injection up to the visit 6 re-supply IVRS contact or up to 21 days after the last study drug injection, whichever came first. Blood sampling collected the day of the visit 6 re-supply IVRS contact will be considered as before titration; 2) the percent change in ApoB from baseline to week 24. Same definition and rules as for the primary endpoint; 3) the percent change in non-HDL-C from baseline to week 24. Same definition and rules as for the primary endpoint; 4) the percent change in total-C from baseline to week 24. Same definition and rules as for the primary endpoint; 5) the percent change in ApoB from baseline to week 12. Same definition and rules as for the percent change in calculated LDL-C from baseline to week 12; 6) the percent change in non-HDL-C from baseline to week 12. Same definition and rules as for the percent change in calculated LDL-C from baseline to week 12; 7) the percent change in total-C from baseline to week 12. Same definition and rules as for the percent change in calculated LDL-C from baseline to week 12; 8) the percent change in calculated LDL-C from baseline to week 52. Definitions and rules are similar to the ones used for the primary endpoint replacing week 24 by week 52; 9) the proportion of patients reaching LDL-C goal at week 24, i.e., LDL-C<70 mg/dL (1.81 mmol/L) in case of prior CVD or <100 mg/dL (2.59 mmol/L) for patients without prior CVD, defined as: (number of patients whose calculated LDL-C value at week 24 reach LDL-C goal/number of patients in the [modified intent-to-treat (mITT population)]*100, using definition and rules used for the primary endpoint; 10) the proportion of patients reaching LDL-C<70 mg/dL (1.81 mmol/L) at week 24; 11) the percent change in Lp(a) from baseline to week 24. Same definition and rules as for the primary endpoint; 12) the percent change in HDL-C from baseline to week 24. Same definition and rules as for the primary endpoint; 13) the percent change in HDL-C from baseline to week 12. Same definition and rules as for the percent change in calculated LDL-C from baseline to week 12; 14) the percent change in Lp(a) from baseline to week 12. Same definition and rules as for the percent change in calculated LDL-C from baseline to week 12; 15) the percent change in fasting TG from baseline to week 24. Same definition and rules as for the primary endpoint; 16) the percent change in fasting TG from baseline to week 12. Same definition and rules as for the percent change in calculated LDL-C from baseline to week 12; 17) the percent change in ApoA-1 from baseline to week 24. Same definition and rules as for the primary endpoint; 18) the percent change in ApoA-1 from baseline to week 12. Same definition and rules as for the percent change in calculated LDL-C from baseline to week 12.

Other secondary efficacy endpoints were: 1) the percent change in calculated LDL-C from baseline to week 78. Definitions and rules are similar to the ones used for the primary endpoint replacing week 24 by week 78; 2) the proportion of patients reaching LDL-C goal at weeks 12, 52, and 78, i.e., LDL-C<70 mg/dL (1.81 mmol/L) in case of prior CVD or <100 mg/dL (2.59 mmol/L) for patients without prior CVD; 3) the proportion of patients reaching LDL-C<100 mg/dL (2.59 mmol/L) at week 24; 4) the proportion of patients reaching LDL-C<100 mg/dL (2.59 mmol/L) at week 12; 5) the proportion of patients reaching LDL-C<70 mg/dL (1.81 mmol/L) at week 12; 6) the absolute change in calculated LDL-C (mg/dL and mmol/L) from baseline to weeks 12, 24, 52, and 78; 7) the percent change in ApoB, non-HDL-C, total-C, Lp(a), HDL-C, fasting TG, and ApoA-1 from baseline to weeks 52 and 78; 8) the change in ratio ApoB/ApoA-1 from baseline to weeks 12, 24, 52, and 78; 9) the proportion of patients with ApoB <80 mg/dL (0.8 g/L) at weeks 12, 24, 52, and 78; 10) the proportion of patients with non-HDL-C<100 mg/dL at weeks 12, 24, 52, and 78; 11) the proportion of patients with calculated LDL-C<70 mg/dL (1.81 mmol/L) and/or ≥50% reduction in calculated LDL-C (if calculated LDL-C≥70 mg/dL [1.81 mmol/L]) at weeks 12, 24, 52, and 78.

Other endpoints were: 1) anti-Alirocumab antibody status (positive/negative) and titers assessed throughout the study; 2) the percent change in high sensitivity C-reactive protein (hs-CRP) from baseline to weeks 24, 52, and 78; 3) the absolute change in HbA1c (%) from baseline to weeks 24, 52, and 78; and 4) response of each EQ-5D item, index score, and change of index score from baseline through week 52.

### Study Visits

The following visits were scheduled:

At Visit 1/Screening/Day −14 to −8; Visit 2/Screening/Day −7 (+/−3 days); Visit 3/Baseline/Week 0/Day 1; Visit 4/Week 4/Day 29 (+/−7 days); Visit 6/Week 12/Day 85 (+/−3 days); Visit 7/Week 16/Day 113 (+/−7 days); Visit 8/Week 24/Day 169 (+/−3 days)/Primary Endpoint Assessment; Visit 9/Week 36/Day 253 (+/−7 days); Visit 10/Week 52/Day 365 (+/−5 days); Visit 11/Week 64/Day 449 (+/−7 days); Visit 12/Week 78/Day 547 (+/−5 days); and the End of Study/Visit 13/Week 86/Day 603 (+/−7 days).

Medical/surgical history, medication history, demographics, height, hepatitis B surface antigen, and serum pregnancy testing were performed for the purpose of determining study eligibility or characterizing the baseline population.

All laboratory samples were collected before the dose of study drug was administered.

Blood samples for lipid panels should be collected in the morning, in fasting condition (i.e., overnight at least 10 hours fast, only water, and refrain from smoking) for all clinic visits. Alcohol consumption within 48 hours, and intense physical exercise and smoking within 24 hours preceding blood sampling were discouraged. Note: if the patient was not in fasting condition, the lipid blood samples were collected and a new appointment was scheduled the day after (or as close as possible to this date), with a reminder for the patient to be fasted.

### Sample Size and Power Considerations

A total sample size of 45 patients (30 in alirocumab and 15 in placebo) will have 95% power to detect a difference in mean percent change in LDL-C of 30% with a 0.05 two-sided significance level; assuming a common standard deviation of 25% and that all 45 patients have an evaluable primary endpoint.

To meet regulatory requirements across the program, the sample size was increased to 126 patients on alirocumab, with the intent to understand safety in a larger population. In order to have at least 126 patients on alirocumab treated for 12 months in this study, and assuming a drop-out rate of 10% over the first 3-month period and a drop-out rate of 20% over the remaining 9-month period, the final total sample size was increased and rounded to 250 patients, with a randomization ratio 2:1 (alirocumab: 167, placebo: 83).

### Analysis Populations

### Intent-to-Treat Population

The randomized population included all randomized patients, and was analyzed according to the treatment allocated by randomization.

The ITT population (also known as the full analysis set [FAS]) was defined as all randomized patients who had an evaluable primary endpoint. The endpoint was evaluable when the following two conditions were met: 1) availability of a baseline calculated LDL-C value; and 2) availability of at least 1 calculated LDL-C value within 1 of the analysis windows up to week 24.

Patients in the ITT population were analyzed according to the treatment group allocated by randomization (i.e., as-randomized treatment group).

### Modified Intent-to-Treat

The mITT population was defined as the all randomized population who took at least 1 dose or part of a dose of study drug and had an evaluable primary endpoint. The endpoint was considered as evaluable (i.e. efficacy treatment period) when both of the following conditions were met: 1) availability of a baseline calculated LDL-C value; and 2) availability of at least 1 calculated LDL-C value during the efficacy treatment period and within one of the analysis windows up to week 24. The efficacy treatment period is defined as the time from the first double-blind study drug injection up to 21 days after the last double-blind study drug injection.

Patients in the mITT population were analyzed according to the treatment group allocated by randomization.

### Safety Analysis Set

The safety population considered for safety analyses was the randomized population who received at least 1 dose or part of a dose of study drug. Patients were analyzed according to the treatment actually received (i.e. as-treated treatment group, placebo or alirocumab).

### Results

### Description of Study Populations

A total of 249 patients were randomized (82 to the placebo group and 167 to the alirocumab group) in this study. One patient in the placebo group was randomized but did not receive study treatment due to the reason of withdrew consent prior to receiving the first IMP injection. Therefore, the patient was excluded from the safety population. Two patients among the randomized patients (the one in the placebo group above and one in the alirocumab group) were excluded from the ITT and mITT populations due to lack of post-baseline LDL-C assessments.

TABLE 14
Analysis Populations

| | | Alirocumab 75 | |
|---|---|---|---|
| | Placebo | Q2W/Up150 Q2W | All |
| | (N = 82) | (N = 167) | (N = 249) |
| Randomized | 82 (100%) | 167 (100%) | 249 (100%) |
| population | | | |
| Efficacy population: | | | |
| Intent-to-Treat (ITT) | 81 (98.8%) | 166 (99.4%) | 247 (99.2%) |
| Modified Intent-to- | 81 (98.8%) | 166 (99.4%) | 247 (99.2%) |
| Treat (mITT) | | | |
| Quality-of-life | 80 (97.6%) | 164 (98.2%) | 244 (98.0%) |
| population | | | |
| Anti-alirocumab | 77 (93.9%) | 166 (99.4%) | 243 (97.6%) |
| antibody population | | | |
| Safety population | 81 (98.8%) | 167 (100%) | 248 (99.6%) |

Note:
The safety, and anti-alirocumab antibody population patients are tabulated according to treatment actually received (as treated). For the other populations, patients are tabulated according to their randomized treatment

In the alirocumab group, among the 158 patients who received at least one injection after Week 12, 61 (38.6%) patients received automatic up-titration at Week 12 in a blinded manner from alirocumab 75 mg Q2W to 150 mg Q2W.

### Subject Dispositions

As of the first-step analyses data cut-off date, patient status is presented below for the 249 randomized patients: 1) 0 (0.0%) patients completed the 78-week double-blind treatment period, due to ongoing patients not yet reaching the week 78 visit; 2) 234 (94.0%) patients were still treatment-ongoing: 78 (95.1%) in the placebo group and 156 (93.4%) in the alirocumab group; 3) 9 (3.6%) randomized and treated patients prematurely discontinued study treatments before Week 24: 1 (1.2%) in the placebo group and 8 (4.8%) in the alirocumab group. 4 (1.6%) patients prematurely terminated study treatments due to adverse events: 0 in the placebo group vs. 4 (2.4%) in the alirocumab group. 3 (1.2%) patients prematurely terminated study treatments due to poor protocol compliance: 1 (1.2%) in the placebo group and 2 (1.2%) in the alirocumab group. 2 (0.8%) patients prematurely terminated study treatments due to various other reasons: 0 in the placebo group vs. 2 (1.2%) in the alirocumab group; 4) 13 (5.2%) randomized and treated patients prematurely discontinued study treatments before Week 52: 2 (2.4%) in the placebo group and 11 (6.6%) in the alirocumab group. 5 (2.0%) patients prematurely terminated study treatments due to adverse events: 0 in the placebo group vs. 5 (3.0%) in the alirocumab group. 3 (1.2%) patients prematurely terminated study treatments due to poor protocol compliance: 1 (1.2%) in the placebo group and 2 (1.2%) in the alirocumab group. 5 (0.8%) patients prematurely terminated study treatments due to various other reasons: 1 (1.2%) in the placebo group and 4 (2.4%) in the alirocumab group; 5) 14 (5.6%) patients prematurely terminated study treatments before completing the 78-week treatment period: 3 (3.7%) in the placebo group and 11 (6.6%) in the alirocumab group. 6 (2.4%) patients prematurely terminated study treatments due to adverse events: 1 (1.2%) in the placebo group and 5 (3.0%) in the alirocumab group. 3 (1.2%) patients prematurely terminated study treatments due to poor protocol compliance: 1 (1.2%) in the placebo group and 2 (1.2%) in the alirocumab group. 5 (2.0%) patients prematurely terminated study treatments due to various other reasons: 1 (1.2%) in the placebo group and 4 (2.4%) in the alirocumab group.

The following table provides the availability of LDL-C values over time. At Week 24, the primary efficacy endpoint was available for 78 (96.3%) patients in the placebo group and 157 (94.5%) in the alirocumab group. There were 77 (95.1%) on-treatment assessments and 1 (1.2%) off-treatment assessments in the placebo group, as compared with 155 (93.4%) on-treatment assessments and 2 (1.2%) off-treatment assessments in the alirocumab group. At Week 52, the key secondary efficacy endpoint was available for 78 (96.3%) patients in the placebo and 158 (95.2%) patients in the alirocumab groups.

TABLE 15
Calculated LDL-C Availability over Time - ITT Population

| | | | | Alirocumab 75 | | | |
|---|---|---|---|---|---|---|---|
| | Placebo | | | Q2W/Up150 Q2W | | | |
| | (N = 81) | | | (N = 166) | | | |
| | On- | Post- | | On- | Post- | | |
| Calculated | treatment | treatment | Missing | treatment | treatment | Missing | |
| LDL-C | value | value | value | value | value | value | |
| WEEK 4 | 79 (97.5%) | 0 | 2 (2.5%) | 162 (97.6%) | 0 | 4 | (2.4%) |
| WEEK 8 | 79 (97.5%) | 0 | 2 (2.5%) | 156 (94.0%) | 0 | 10 | (6.0%) |
| WEEK 12 | 76 (93.8%) | 0 | 5 (6.2%) | 151 (91.0%) | 1 (0.6%) | 14 | (8.4%) |
| WEEK 16 | 77 (95.1%) | 0 | 4 (4.9%) | 149 (89.8%) | 3 (1.8%) | 14 | (8.4%) |
| WEEK 24 | 77 (95.1%) | 1 (1.2%) | 3 (3.7%) | 155 (93.4%) | 2 (1.2%) | 9 | (5.4%) |
| WEEK 36 | 73 (90.1%) | 0 | 8 (9.9%) | 153 (92.2%) | 2 (1.2%) | 11 | (6.6%) |
| WEEK 52 | 78 (96.3%) | 0 | 3 (3.7%) | 155 (93.4%) | 3 (1.8%) | 8 | (4.8%) |

An on-treatment value was obtained after the first study treatment injection and within 21 days after the last study treatment injection.
A post-treatment value was obtained more than 21 deays after the last study treatment injection.

The primary endpoint was missing for 12 (4.9%) patients at Week 24. At the Week 24 visit, the reasons for missing values were as follows: 1) 4 subjects with samples not obtained due to earlier study discontinuation; 2) 2 subjects were still ongoing, but Week 24 LDL-C was not done; 3) 6 samples were obtained at Week 24, but the LDL-C could not be calculated (5 with TGs>400 mg/dL and measured LDL-C reported, 1 with >400 mg/dL but measured LDL-C not reported).

### Demographic and Baseline Characteristics

Overall, demographic characteristics, baseline disease characteristics, baseline efficacy lipid parameters, LMT history and background LMT use were homogeneous between patients randomized to the alirocumab group and patients randomized to the placebo group (see Table 16). Particularly, the mean baseline LDL-C in the alirocumab group was 134.6 mg/dL (SD=41.1 mg/dL) compared to that in the placebo group being 134.0 mg/dL (SD=41.4 mg/dL) with an overall mean of 134.4 mg/dL (SD=41.1 mg/dL). One potentially notable exception is the difference observed in baseline BMI, with a mean BMI of 28.6 kg/m2 (SD=4.6 kg/m2) in the alirocumab group compared to 27.7 kg/m2 (SD=4.7 kg/m2) in the placebo group.

TABLE 16
Baseline Characteristics of FHII Patient Population

| | Alirocumab | | Placebo | |
|---|---|---|---|---|
| Characteristic | (N = 167) | | (N = 82) | |
| Age, mean (SD), yrs | 53.2 | (12.9) | 53.2 | (12.5) |
| Diagnosis of heFH^{†}, % (n) | | | | |
| Genotyping | 70.1% | (117) | 81.7% | (67) |
| Clinical criteria | 29.9% | (50) | 18.3% | (15) |
| Male | 51.5% | (86) | 54.9% | (45) |
| Race, white | 98.2% | (164) | 97.6% | (80) |
| BMI, mean (SD), kg/m² | 28.6 | (4.6) | 27.7 | (4.7) |
| CHD history | 34.1% | (57) | 37.8% | (31) |
| CHD risk equivalents^{†} | 9.0% | (15) | 4.9% | (4) |
| Current smoker | 21.6% | (36) | 15.9% | (13) |
| Hypertension | 34.1% | (57) | 29.3% | (24) |
| Type 2 diabetes | 4.2% | (7) | 3.7% | (3) |

% (N) of patients unless stated. All pts on background of max tolerated statin ± other lipid-lowering therapy.
^{†}Diagnosis of heFH must be made either by genotyping or by clinical criteria. For those patients not genotyped, the clinical diagnosis may be based on either the Simon Broome criteria for definite FH or the WHO/Dutch Lipid Network criteria with a score of >8 points.

TABLE 17
Disease Characteristics and Other Relevant Baseline Data - Randomized Population

| | | Alirocumab 75 | | P Value |
|---|---|---|---|---|
| | Placebo | Q2W/Up150 Q2W | All | vs. |
| | (N = 82) | (N = 167) | (N = 249) | Placebo |
| Type of hypercholesterolemia | | | | |
| Heterozygous Familial Hypercholesterolemia | 82 (100%) | 167 (100%) | 249 (100%) | |
| (heFH) | | | | |
| Non-Familial Hypercholesterolemia (non-FH) | 0 | 0 | 0 | |
| Time from hypercholesterolemia diagnosis (years) | | | | |
| Number | 82 | 167 | 249 | 0.4938 |
| Mean (SD) | 12.7 (8.8) | 12.9 (7.9) | 12.8 (8.2) | |
| Median | 10.8 | 12.3 | 11.5 | |
| Min:Max | 0:42 | 0:40 | 0:42 | |
| Confirmation of diagnosis* | | | | |
| By genotyping | 67 (81.7%) | 117 (70.1%) | 184 (73.9%) | |
| By WHO/Simon Broome | 18 (22.0%) | 52 (31.1%) | 70 (28.1%) | |
| Definite/Certain | 18 (22.0%) | 52 (31.1%) | 70 (28.1%) | |

*heFH diagnosis can be confirmed by both genotyping and WHO or Simon Broome criteria.
Note:
p-values comparing baseline data between treatment groups are provided for descriptive purpose, as a screening tool, using Fisher exact test for qualitative data and the asymptotic one-way ANOVA test for Wilcoxon scores (Krukal-Wallis test) for continuous data.

TABLE 18
Background LMT at Randomization - Randomized Population

| | | | Alirocumab 75 | | | | |
|---|---|---|---|---|---|---|---|
| | Placebo | | Q2W/Up150 Q2W | | All | | P = Value |
| | (N = 82) | | (N = 167) | | (N = 249) | | vs. Placebo |
| Any statin | 82 | (100%) | 167 | (100%) | 249 | (100%) | |
| Taking high intensity statin | 72 | (87.8%) | 144 | (86.2%) | 216 | (86.7%) | 0.8434 |
| Atorvastatin daily dose (mg) | | | | | | | |
| 10 | 2 | (2.4%) | 2 | (1.2%) | 4 | (1.6%) | |
| 20 | 0 | | 8 | (4.8%) | 8 | (3.2%) | |
| 40 | 13 | (15.9%) | 27 | (16.2%) | 40 | (16.1%) | |
| 80 | 16 | (19.5%) | 28 | (16.8%) | 44 | (17.7%) | |
| Other doses | 1 | (I.2%) | 0 | | 1 | (0.4%) | |
| Rosuvastatin daily dose (mg) | | | | | | | |
| 5 | 1 | (1.2%) | 1 | (0.6%) | 2 | (0.8%) | |
| 10 | 2 | (2.4%) | 4 | (2.4%) | 6 | (2.4%) | |
| 20 | 8 | (9.8%) | 30 | (18.0%) | 38 | (15.3%) | |
| 40 | 33 | (40.2%) | 59 | (35.3%) | 92 | (36.9%) | |
| Other doses | 1 | (1.2%) | 1 | (0.6%) | 2 | (0.8%) | |
| Simvastatin daily dose (mg) | | | | | | | |
| 10 | 1 | (1.2%) | 0 | | 1 | (0.4%) | |
| 20 | 1 | (1.2%) | 3 | (1.8%) | 4 | (1.6%) | |
| 40 | 0 | | 3 | (1.8%) | 3 | (1.2%) | |
| 80 | 3 | (3.7%) | 1 | (0.6%) | 4 | (1.6%) | |

| | | | |
|---|---|---|---|
| Other doses | 0 | 0 | 0 |

Any LMT other than statins*

| | | | | | | |
|---|---|---|---|---|---|---|
| 57 | (69.5%) | 117 | (70.1%) | 174 | (69.9%) | 1.0000 |

Any LMT other than nutraceuticals

| | | | | | | |
|---|---|---|---|---|---|---|
| | 54 | (65.9%) | 115 | (68.9%) | 169 | (67.9%) |
| Ezetimibe | 53 | (64.6%) | 112 | (67.1%) | 165 | (66.3%) |
| Nutraceuticals | 7 | (8.5%) | 8 | (4.8%) | 15 | (6.0%) |

Note:
p = values comparing baseline data between treatment groups are provided for descriptive purpose, as a screening tool, using Fisher exact test.
*in combination with statins or not.

TABLE 19
Cardiovascular History and Risk Factors Breakdown

| | Alirocumab | | Placebo | |
|---|---|---|---|---|
| Characteristic | (N = 323) | | (N = 163) | |
| CHD history | 34.1% | (57) | 37.8% | (31) |
| Acute MI | 16.2% | (27) | 17.1% | (14) |
| Silent MI | 0.6% | (1) | 2.4% | (2) |
| Unstable angina | 9.0% | (15) | 9.8% | (8) |
| Coronary revasc. | 27.5% | (46) | 29.3% | (24) |
| Other clinically significant CHD | 16.2% | (27) | 20.7% | (17) |
| CHD risk equivalents | 9.0% | (15) | 4.9% | (4) |
| Ischemic stroke | 3.0% | (5) | 1.2% | (1) |
| Peripheral arterial disease | 3.0% | (5) | 1.2% | (1) |
| Moderate CKD | 1.2% | (2) | 1.2% | (1) |
| Diabetes + 2 or more risk factors | 3.0% | (5) | 2.4% | (2) |

% (N) of patients unless stated. All pts on background of max tolerated statin ± other lipid-lowering therapy

TABLE 20
Lipid Efficacy Parameters at Baseline - Quantitative
Summary in Conventional Units - Randomized Population

| | | | Alirocumab 75 | | | | P Value |
|---|---|---|---|---|---|---|---|
| | Placebo | | Q2W/Up150 Q2W | | All | | vs. |
| | (N = 82) | | (N = 167) | | (N = 249) | | Placebo |
| Calculated LDL-C (mg/dL) | | | | | | | |
| Number | 82 | | 167 | | 249 | | 0.8507 |
| Mean (SD) | 134.0 | (41.4) | 134.6 | (41.1) | 134.4 | (41.1) | |
| Median | 126.0 | | 128.0 | | 126.0 | | |
| Q1:Q3 | 109.0:151.0 | | 107.0:154.0 | | 108.0:151.0 | | |
| Min:Max | 74:295 | | 58:303 | | 58:303 | | |
| Measured LDL-C (mg/dL) | | | | | | | |
| Number | 70 | | 149 | | 219 | | 0.6375 |
| Mean (SD) | 130.2 | (36.6) | 132.6 | (40.6) | 131.8 | (39.3) | |
| Median | 125.5 | | 126.0 | | 126.0 | | |
| Q1:Q3 | 104.0:145.0 | | 104.0:149.0 | | 104.0:147.0 | | |
| Min:Max | 71:249 | | 49:310 | | 49:310 | | |
| HDL-C (mg/dL) | | | | | | | |
| Number | 82 | | 167 | | 249 | | 0.4437 |
| Mean (SD) | 54.2 | (15.7) | 52.6 | (15.7) | 53.1 | (15.7) | |
| Median | 51.0 | | 50.0 | | 51.0 | | |
| Q1:Q3 | 42.0:63.0 | | 42.0:61.0 | | 42.0:62.0 | | |
| Min:Max | 25:103 | | 24:110 | | 24:110 | | |
| Total-C (mg/dL) | | | | | | | |
| Number | 82 | | 167 | | 249 | | 0.9589 |
| Mean (SD) | 211.7 | (45.6) | 211.6 | (45.8) | 211.6 | (45.6) | |
| Median | 200.0 | | 205.0 | | 202.0 | | |
| Q1:Q3 | 179.0:237.0 | | 178.0:242.0 | | 179.0:239.0 | | |
| Min:Max | 133:376 | | 123:391 | | 123:391 | | |
| Non-HDL-C (mg/dL) | | | | | | | |
| Number | 82 | | 167 | | 249 | | 0.8208 |
| Mean (SD) | 157.5 | (43.7) | 159.0 | (44.8) | 158.5 | (44.4) | |
| Median | 150.5 | | 147.0 | | 149.0 | | |
| Q1:Q3 | 129.0:170.0 | | 127.0:181.0 | | 127.0:177.0 | | |
| Min:Max | 93:320 | | 76:326 | | 76:326 | | |
| Fasting TGs (mg/dL) | | | | | | | |
| Number | 82 | | 167 | | 249 | | 0.6593 |
| Mean (SD) | 116.6 | (56.8) | 123.2 | (69.3) | 121.0 | (65.4) | |
| Median | 100.5 | | 105.0 | | 104.0 | | |
| Q1:Q3 | 81.0:136.0 | | 81.0:144.0 | | 81.0:141.0 | | |
| Min:Max | 47:366 | | 46:581 | | 46:581 | | |
| Apo-B (mg/dL) | | | | | | | |
| Number | 81 | | 167 | | 248 | | 0.9533 |
| Mean (SD) | 107.7 | (23.9) | 107.9 | (27.4) | 107.9 | (26.3) | |
| Median | 103.0 | | 102.0 | | 102.0 | | |
| Q1:Q3 | 91.0:116.0 | | 91.0:122.0 | | 91.0:121.0 | | |
| Min:Max | 74:187 | | 57:208 | | 57:208 | | |
| Apo-A1 (mg/dL) | | | | | | | |
| Number | 81 | | 167 | | 248 | | 0.3472 |
| Mean (SD) | 148.9 | (29.6) | 146.3 | (29.4) | 147.2 | (29.4) | |
| Median | 150.0 | | 142.0 | | 144.5 | | |
| Q1:Q3 | 129.0:166.0 | | 127.0:160.0 | | 128.0:162.5 | | |
| Min:Max | 82:223 | | 90:252 | | 82:252 | | |
| Apo-B/Apo-A1 (ratio) | | | | | | | |
| Number | 81 | | 167 | | 248 | | 0.7518 |
| Mean (SD) | 0.8 | (0.2) | 0.8 | (0.2) | 0.8 | (0.2) | |
| Median | 0.7 | | 0.7 | | 0.7 | | |
| Q1:Q3 | 0.6:0.8 | | 0.6:0.9 | | 0.6:0.9 | | |
| Min:Max | 0:1 | | 0:2 | | 0:2 | | |
| Lipoprotein-(a) (mg/dL) | | | | | | | |
| Number | 81 | | 167 | | 248 | | 0.9910 |
| Mean (SD) | 50.9 | (59.7) | 49.8 | (69.2) | 50.2 | (66.1) | |
| Median | 21.0 | | 22.0 | | 22.0 | | |
| Q1:Q3 | 7.0:76.0 | | 8.0:70.0 | | 7.5:75.0 | | |
| Min:Max | 2:232 | | 2:555 | | 2:555 | | |
| Total-C/HDL-C (ratio) | | | | | | | |
| Number | 82 | | 167 | | 249 | | 0.6572 |
| Mean (SD) | 4.2 | (1.3) | 4.3 | (1.5) | 4.3 | (1.5) | |

| | | | |
|---|---|---|---|
| Median | 3.9 | 3.9 | 3.9 |
| Q1:Q3 | 3.3:4.8 | 3.3:5.0 | 3.3:4.9 |
| Min:Max | 2:9 | 2:11 | 2:11 |

Note:
p-values comparing baseline data between treatment groups are provided for descriptive purpose, as a screening tool, using the asymptotic one-way ANOVA test for Wilcoxon scores (Kruskal-Wallis test).

The collection of measured LDL-C was not planned in the initial protocol and was added in an amendment. Therefore, measured LDL-C values are available for fewer patients compared to calculated LDL-C values.

### Extent of Exposure

Exposure to injections was similar across treatment groups with a mean exposure of approximately 58-60 weeks. Alirocumab treated patients were exposed for 2-75.9 weeks and placebo treated patients for 11.6-75.7 weeks. The majority (93.5%:97.5%, alirocumab:placebo, respectively) of patients were treated for more than 52 weeks.

In the alirocumab group, among the 158 patients who received at least one injection after Week 12, 61 (38.6%) patients received automatic up-titration in a blinded manner at Week 12 from alirocumab 75 mg Q2W to 150 mg Q2W. 26 patients were not given the opportunity to up-titrate at Week 12 due to missing Week 8 LDL-C values at the time of the up-titration decision. Of the 26 patients missing the Week 8 LDL-C value, 4 alirocumab patients should have been up-titrated based on the now available Week 8 LDL-C data. The remaining patients were either in the placebo treatment group, or the Week 8 LDL-C visits for the alirocumab patients were below the LDL-C<70 mg/dL cut-off for up-titration.

### Efficacy Analyses

### Primary Efficacy Analysis in the ITT Population

The primary endpoint (percent change in calculated LDL-C from baseline to Week 24) analysis is provided based on a MMRM model on the ITT population, using LS means estimates at Week 24. This repeated measures approach includes all LDL-C values collected on-treatment and off-treatment up to Week 52. At Week 24, 3 (3.7%) patients in the placebo group and 9 (5.4%) patients in the alirocumab group did not have a calculated LDL-C value (Table 15). These missing values were accounted for by the MMRM model.

The primary efficacy analysis showed a statistically significant decrease in percent change from baseline calculated LDL-C at Week 24 in the ITT analysis for the alirocumab treatment group (LS mean=−48.7%) as compared to placebo (LS mean=2.8%). The LS mean difference between the alirocumab treatment group and the placebo treatment groups is −51.4% (p<0.0001). 81.4% of HeFH patients in the alirocumab group achieved the LDL-C goals at 24 weeks, compared to 11.3% for the placebo group.

TABLE 21
Percent Change from Baseline in Calculated LDL-C at Week
24 (ITT Analysis): MMRM Analysis - ITT Population

| | | | Alirocumab 75 | |
|---|---|---|---|---|
| Calculated LDL | Placebo | | Q2W/Up150 Q2W | |
| Cholesterol | (N = 81) | | (N = 166) | |
| Baseline (mmol/L) | | | | |
| Number | 81 | | 166 | |
| Mean (SD) | 3.470 | (1.078) | 3.486 | (1.069) |
| Median | 3.263 | | 3.289 | |
| Min:Max | 1.92:7.64 | | 1.50:7.85 | |
| Baseline (mg/dL) | | | | |
| Number | 81 | | 166 | |
| Mean (SD) | 134.0 | (41.6) | 134.6 | (41.3) |
| Median | 126.0 | | 127.0 | |
| Min:Max | 74:295 | | 58:303 | |
| Week 24 percent change | | | | |
| from baseline (%) | | | | |
| LS Mean (SE) | 2.8 | (2.8) | −48.7 | (1.9) |
| LS mean difference (SE) vs | | | −51.4 | (3.4) |
| Placebo | | | | |

| | |
|---|---|
| 95% CI | (−58.1 to −44.8) |
| p-value vs. Placebo | <.0001 |

Note:
Lean-squares (LS) means, standard errors (SE) and p-value taken from MMRM (mixed-effect model with repeated measures) analysis. The model includes the fixed categorical effects of treatment group, randomization strata as per IVRS, time point, treatment-by-time point interaction, strata-by-time point interaction, as well as the continuous fixed covariates of baseline calculated LDL-C value and baseline value by time-point interaction.
MMRM model and baseline description run on patients with a baseline value and a post-baseline value in at least one of the analysis windows used in the model.
The p-value is followed by a ‘*’ if statistically significant according to the fixed hierarchical approach used to ensure a strong control of the overall type-1 error rate at the 0.05 level.

### Calculated LDL-C Over Time

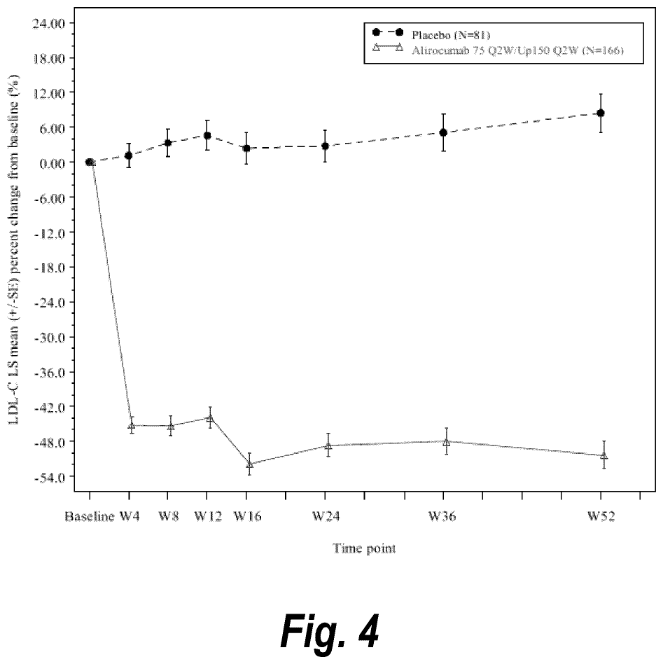

FIG. 4 is a graph that shows the LDL-C LS mean (+/−SE) percent change from baseline over time for the ITT population. Note: Least-squares (LS) means and standard errors (SE) taken from MMRM (mixed-effect model with repeated measures) analysis.

The model includes the fixed categorical effects of treatment group, time point, treatment-by-time point interaction, as well as the continuous fixed covariates of baseline LDL-C value and baseline LDL-C-by-time point interaction.

TABLE 22
Calculated LDL-C Over Time - ITT Population

| | | | | | Alirocumab 75 | | | |
|---|---|---|---|---|---|---|---|---|
| | Placebo | | | | Q2W/Up150 Q2W | | | |
| | (N = 81) | | | | (N = 166) | | | |
| | | | | Percent | | | | Percent |
| | | | Change | change | | | Change | change |
| Calculated | | | from | from | | | from | from |
| LDL-C | Value | | baseline | baseline | Value | | baseline | baseline |
| LS mean (SE) (mmol/L) | | | | | | | | |
| Baseline | 3.470 | (0.120) | NA | NA | 3.486 | (0.083) | NA | NA |
| Week 4 | 3.485 | (0.077) | 0.004 (0.077) | 1.1 (2.0) | 1.924 | (0.054) | −1.56 (0.054) | −45.2 (1.4) |
| Week 8 | 3.561 | (0.090) | 0.081 (0.090) | 3.3 (2.4) | 1.913 | (0.063) | −1.57 (0.063) | −45.3 (1.7) |
| Week 12 | 3.585 | (0.097) | 0.104 (0.097) | 4.6 (2.6) | 1.960 | (0.068) | −1.52 (0.068) | −43.8 (1.8) |
| Week 16 | 3.508 | (0.101) | 0.028 (0.101) | 2.4 (2.7) | 1.649 | (0.071) | −1.83 (0.071) | −51.9 (1.9) |
| Week 24 | 3.537 | (0.103) | 0.057 (0.103) | 2.8 (2.8) | 1.754 | (0.072) | −1.73 (0.072) | −48.7 (1.9) |
| Week 36 | 3.603 | (0.117) | 0.122 (0.117) | 5.1 (3.2) | 1.788 | (0.081) | −1.69 (0.081) | −48.0 (2.2) |
| Week 52 | 3.718 | (0.125) | 0.237 (0.125) | 8.4 (3.3) | 1.708 | (0.088) | −1.77 (0.088) | −50.3 (2.3) |
| Week 64 | 3.601 | (0.107) | | | 1.657 | (0.075) | | |
| Week 78 | 3.574 | (0.109) | | | 1.806 | (0.076) | | |
| LS mean (SE) (mg/dL) | | | | | | | | |
| Baseline | 134.0 | (4.6) | NA | NA | 134.6 | (3.2) | NA | NA |
| Week 4 | 134.6 | (3.0) | 0.2 (3.0) | 1.1 (2.0) | 74.3 | (2.1) | −60.1 (2.1) | −45.2 (1.4) |
| Week 8 | 137.5 | (3.5) | 3.1 (3.5) | 3.3 (2.4) | 73.9 | (2.4) | −60.5 (2.4) | −45.3 (1.7) |
| Week 12 | 138.4 | (3.7) | 4.0 (3.7) | 4.6 (2.6) | 75.7 | (2.6) | −58.7 (2.6) | −43.8 (1.8) |
| Week 16 | 135.5 | (3.9) | 1.1 (3.9) | 2.4 (2.7) | 63.7 | (2.7) | −70.7 (2.7) | −51.9 (1.9) |
| Week 24 | 136.6 | (4.0) | 2.2 (4.0) | 2.8 (2.8) | 67.7 | (2.8) | −66.7 (2.8) | −48.7 (1.9) |
| Week 36 | 139.1 | (4.5) | 4.7 (4.5) | 5.1 (3.2) | 69.0 | (3.1) | −65.3 (3.1) | −48.0 (2.2) |
| Week 52 | 143.6 | (4.8) | 9.2 (4.8) | 8.4 (3.3) | 65.9 | (3.4) | −68.4 (3.4) | −50.3 (2.3) |
| Week 64 | 139.0 | (4.1) | | | 64.0 | (2.9) | | |
| Week 78 | 138.0 | (4.2) | | | 69.7 | (2.9) | | |

* Baseline is described using means and standard errors.
Note:
Least-squares (LS) means, standard errors (SE) and p-value taken from MMRM (mixed-effect model with repeated measures) analysis. The model includes the fixed categorical effects of treatment group, randomization strata as per IVRS, time point, treatment-by-time point interaction, strata-by-time point interaction, as well as the continuous fixed covariates of baseline LDL-C value and baseline LDL-C value by time point interaction.
MMRM model and baseline description run on patients with a baseline value and a post-baseline value in at least one of the analysis windows used in the model.

### Sensitivity to Serious GCP Non-Compliance

There was no site with serious GCP non-compliance in this study.

### Key Secondary Efficacy Analysis

The following table summarizes analysis results on all key secondary endpoints in the hierarchical order for statistical testing at the 0.05 significance level. This study has achieved statistically significant effects in favor of the alirocumab treated patients for all but the last one in the hierarchy (i.e., Apo A-1—Percent change from baseline to Week 12) of the key secondary efficacy endpoints.

For clarification, the ITT analysis is defined for patients in the ITT population and includes all endpoint assessments in an analysis window, regardless of study treatment dosing status (i.e. includes post-treatment assessments). The on-treatment analysis is defined for patients in the mITT population and includes all endpoint assessments from the first double-blind study drug injection up to the day of last injection+21 days (i.e. includes assessments in the efficacy treatment period).

TABLE 23
Summary of Key Secondary Efficacy Endpoints

| | | Alirocumab | | |
|---|---|---|---|---|
| Endpoint/Analysis | Placebo Result | Result | Comparison | P-value |
| 1. LDL-C at WK 24 - ITT | LS mean: 2.8% | LS mean: −48.7% | Diff: −51.4% | <.0001 |
| analysis | | | | |
| 2. LDL-C at WK 24 - on- | LS mean: 2.7% | LS mean: −49.4% | Diff: −52.2% | <.0001 |
| treatment analysis | | | | |
| 3. LDL-C at WK 12 - ITT | LS mean: 4.6% | LS mean: −43.8% | Diff: −48.4% | <.0001 |
| analysis | | | | |
| 4. LDL-C at WK 12 - on- | LS mean: 4.6% | LS mean: −44.2% | Diff: −48.8% | <.0001 |
| treatment analysis | | | | |
| 5. Apo B at WK 24 - ITT | LS mean: −3.5% | LS mean: −42.8% | Diff: −39.3% | <.0001 |
| analysis | | | | |
| 6. Apo B at WK 24 - on- | LS mean: −3.5% | LS mean: −43.2% | Diff: −39.8% | <.0001 |
| treatment analysis | | | | |
| 7. Non-HDL-C at WK 24 - | LS mean: 3.1% | LS mean: −42.6% | Diff: −45.7% | <.0001 |
| ITT analysis | | | | |
| 8. Non-HDL-C at WK 24 - | LS mean: 3.1% | LS mean: −43.2% | Diff: −46.4% | <.0001 |
| on-treatment analysis | | | | |
| 9. Total Cholesterol at WK | LS mean: 2.1% | LS mean: −30.6% | Diff: −32.8% | <.0001 |
| 24 - ITT analysis | | | | |
| 10. Apo B at WK 12- ITT | LS mean: −0.9% | LS mean: −35.4% | Diff: −34.5% | <.0001 |
| analysis | | | | |
| 11. Non-HDL-C at WK 12- | LS mean: 4.1% | LS mean: −37.9% | Diff: −42.0% | <.0001 |
| ITT analysis | | | | |
| 12. Total Cholesterol at | LS mean: 3.4% | LS mean: −26.6% | Diff: −29.9% | <.0001 |
| WK 12 - ITT analysis | | | | |
| 13. LDL-C at WK 52 - ITT | LS mean: 8.4% | LS mean: −50.3% | Diff: −58.8% | <.0001 |
| analysis | | | | |
| 14. Very High CV LDL-C <70 | Proportion = 1.3% | Proportion = 81.4% | Odds Ratio = 52.2 | <.0001 |
| mg/dL OR High CV LDL-C <100 | | | | |
| mg/dL at WK 24 - ITT | | | | |
| analysis | | | | |
| 15. Very High CV LDL-C <70 | Proportion = 1.6% | Proportion = 82.1% | Odds Ratio = 53.3 | <.0001 |
| mg/dL OR High CV LDL-C <100 | | | | |
| mg/dL at WK 24 - on- | | | | |
| treatment analysis | | | | |
| 16. LDL-C <70 mg/dL at | Proportion = 1.2% | Proportion = 68.2% | Odds Ratio = 239.7 | <.0001 |
| WK 24 - ITT analysis | | | | |
| 17. LDL-C <70 mg/dL at | Proportion = 1.3% | Proportion = 68.8% | Odds Ratio = 240.6 | <.0001 |
| WK 24 - on-treatment | | | | |
| analysis | | | | |
| 18. Lp(a) at WK 24 - ITT | LS mean: −10.0% | LS mean: −30.3% | Diff: −20.3% | <.0001 |
| analysis | | | | |
| 19. HDL-C at WK 24 - ITT | LS mean: −0.8% | LS mean: 6.0% | Diff: 6.8% | 0.0009 |
| analysis | | | | |
| 20. Fasting Triglycerides at | LS mean: 0.4% | LS mean: −10.5% | Diff: −10.9% | 0.0017 |
| WK 24 - ITT analysis | | | | |
| 19. Apo A-1 at WK 24 - ITT | LS mean: −1.6% | LS mean: 2.8% | Diff: 4.4% | 0.0062 |
| analysis | | | | |
| 20. Lp(a) at WK 12 - ITT | LS mean: −5.6% | LS mean: −24.7% | Diff: −19.1% | <.0001 |
| analysis | | | | |
| 21. HDL-C at WK 12 - ITT | LS mean: 1.7% | LS mean: 6.0% | Diff: 4.3% | 0.0147 |
| analysis | | | | |
| 22. Fasting Triglycerides at | LS mean: 0.9% | LS mean: −8.0% | Diff: −8.9% | 0.0258 |
| WK 12 - ITT analysis | | | | |
| Apo A-1 at WK 12 - ITT | LS mean: −1.9% | LS mean: 0.4% | Diff: 2.3% | 0.1475 |
| analysis | | | | |

Hierarchical testing terminated

All the key secondary efficacy endpoints, except for percent change in Apo A-1 from baseline to Week 12 in ITT population, achieved statistically significant effects in favor of the alirocumab treated patients according to the hierarchical testing procedure.

The key secondary efficacy analysis for percent change from baseline of calculated LDL-C to week 24 in the mITT population (on-treatment analysis) showed consistent results with the ITT analysis with a statistically significant decrease in calculated LDL-C in the alirocumab treatment group (LS mean=−49.4%) as compared to placebo (LS mean=2.7%). The LS mean treatment difference between the alirocumab-treated patients and the placebo-treated patients is −52.2% (p<0.0001). Indeed, few patients had LDL-C values collected post-treatment (i.e., more than 21 days after last injection) at Week 24: 1 patient (1.2%) in the placebo group and 2 patients (1.2%) in the alirocumab group.

The decrease in percent change in Apo A-1 from baseline to Week 24 in the ITT analysis was non-statistically significant: LS mean versus baseline was 0.4% in the alirocumab group and −1.9% in the placebo group (LS mean difference vs. placebo of 2.3%, p=0.1475).

### Calculated Ldl-C Over Time (Includes Observed Data)

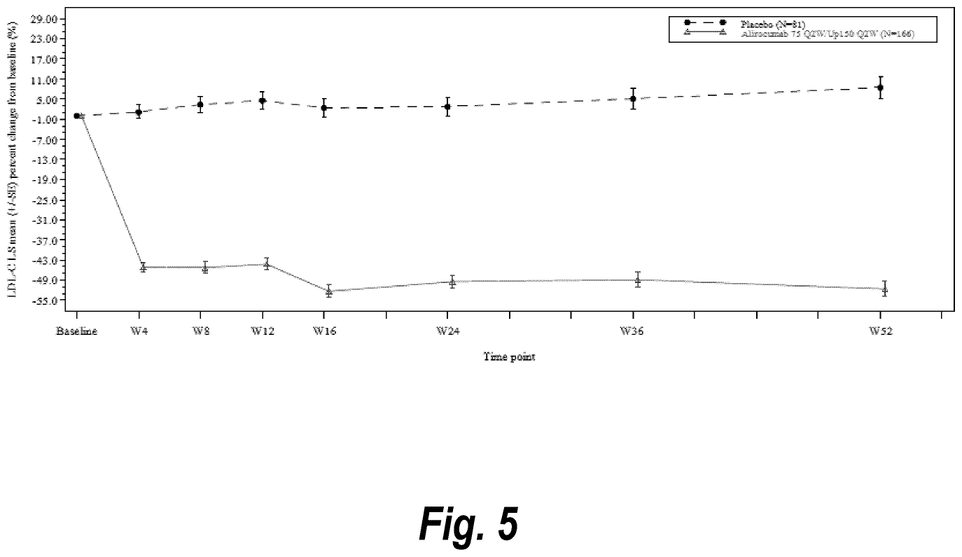

FIG. 5 is a graph that shows the LDL-C LS mean (+/−SE) percent change from baseline during the efficacy treatment period over time for the mITT Population.

### SUMMARY

Overall, demographic characteristics, baseline disease characteristics, baseline efficacy lipid parameters, LMT history and background LMT use were comparable between patients randomized to the alirocumab group and patients randomized to the placebo group. Particularly, the mean (SD) baseline LDL-C in the alirocumab group was 134.6 (41.1) mg/dL compared to that in the placebo group being 134.0 (41.4) mg/dL.

The primary efficacy endpoint and all the key secondary endpoints, except for percent change in Apo A-1 from baseline to Week 12 in ITT population (ITT analysis), achieved statistically significant benefit in favor of Alirocumab-treated patients according to the hieracrchical testing procedure.

### Summary Safety Results

A total of 248 patients were randomized and received at least a partial dose of study treatment (Safety Population). Below is a high-level summary of adverse events and events of interest.

TABLE 24
Overview of Adverse Event Profile: Treatment
Emergent Adverse Events - Safety Population

| | | Alirocumab 75 |
|---|---|---|
| | Placebo | Q2W/Up150 Q2W |
| | (N = 81) | (N = 167) |
| Patients with any TEAE | 62 (76.5%) | 117 (70.1%) |
| Patients with any treatment | 7 (8.6%) | 10 (6.0%) |
| emergent SAE | | |
| Patients with any TEAE leading | 0 | 0 |
| to death | | |
| Patients with any TEAE leading | 1 (1.2%) | 5 (3.0%) |
| to permanent treatment | | |
| discontinuation | | |

TEAE: Treatment emergent adverse event,
SAE: Serious adverse event
n(%) = number and percentage of patients with at least one TEAE

Treatment-emergent SAEs occurred in a total of 17 patients, specifically 10 (6.0%) patients in the alirocumab treatment group and 7 (8.6%) patients in the placebo treatment group. There were no more than 2 reports in any SOC for either treatment group and no individual SAE was reported more than once in either treatment group.

No patient deaths were reported at the time of this first-step analysis.

A total of 6 patients prematurely discontinued study treatment due to a TEAE. Specifically, 5 (3.0%) patients in the alirocumab treatment group discontinued treatment early for rectal adenocarcinoma, diarrhoea, nausea, angioedema, asthenia, and alanine aminotransferase increased. One (1.2%) patient in the placebo treatment group discontinued due to syncope.

TEAEs occurred in 117 (70.1%) patients in the alirocumab treatment group and 62 (76.5%) patients in the placebo treatment group. The TEAEs that occurred in ≥5% of patients in any treatment group are: injection site reaction (10.8% vs. 7.4% in alirocumab and placebo group, respectively), headache (8.4% vs. 8.6% in alirocumab and placebo group, respectively), myalgia (6.0% vs. 6.2% in alirocumab and placebo group, respectively), and diarrhoea (5.4% vs. 1.2% in alirocumab and placebo group, respectively).

For TEAEs of special interest (AESIs), results are presented by pre-defined SMQ preferred term groupings.

Treatment-emergent injection site reactions (ISRs) occurred in 18 (10.8%) patients in the alirocumab treatment group and 6 (7.4%) patients in the placebo treatment group. None of the AEs were serious.

General Allergic TEAEs, identified through the MedDRA SMQ of “Hypersensitivity” occurred in 17 (10.2%) patients in the alirocumab treatment group and 6 (7.4%) patients in the placebo treatment group. None of the AEs were serious.

Treatment-emergent neurologic disorders occurred in 7 (4.2%) patients in the alirocumab treatment group and 2 (2.5%) patients in the placebo treatment group. In the alirocumab group, the PTs were: hypoaesthesia in 4 (2.4%) patients, paraesthesia in 2 (1.2%), and balance disorder in 1 (0.6%). None of the AEs were serious.

Treatment-emergent neurocognitive disorders occurred in 0 (0.0%) patients in the alirocumab treatment group and 1 (1.2%) patients in the placebo treatment group. The AE was not serious.

A total of 9 (5.4%) patients in the alirocumab treatment group and 0 (0.0%) patients in the placebo treatment group had 2 consecutive calculated LDL-C measurements below 25 mg/dL. For those patients with 2 consecutive calculated LDL-C measurements below 25 mg/dL, TEAEs occurred in 3 (33.3%) patients in the alirocumab treatment. The PTs were: influenza, influenza like illness, and nasopharyngitis. None of these AEs were serious, nor were they AESIs.

### Conclusion

The following conclusions can be drawn from this early review of the study data: 1) the study achieved the primary efficacy endpoint with a statistically significant reduction in calculated LDL-C in the alirocumab treated patients; 2) this study also achieved all of the key secondary efficacy endpoints, except for the last endpoint (Apo A-1 at Week 12 in the ITT population (ITT analysis)); and 3) based on the available data at the time of this first step analysis, subcutaneous administration of alirocumab to patients with heterozygous familial hypercholesterolemia and an LDL-C>70 mg/dL or LDL-C>100 mg/dL, depending on history of MI or stroke at baseline, was generally safe and well tolerated.

Summary of Pooled Data from FH I and FH II Studies

From the pooled data of the FHI and FHII studies the following conclusions can be drawn: 1) self-administered alirocumab produced significantly greater LDL-C reductions vs. placebo after 24 weeks (LS mean difference of 51.4-57.9%); 2) the majority of patients (>70%) achieved their LDL-C goals at Week 24; 3) LDL-C reductions of 47.1-50.3% at Week 52 were achieved with alirocumab; 4) mean LDL-C levels of 1.7-1.9 mmol/L (65.9-74.3 mg/dL) at Week 52 were achieved with alirocumab; 5) approximately 50% of patients did not require uptitration to alirocumab 150 mg Q2W suggesting that 75 mg Q2W may be sufficient for many patients; and 6) TEAEs occurred in a similar frequency in the alirocumab and placebo arms.

Specifically, the combined data of the FHI and FHII studies shows that alirocumab produced a significant reduction in LDL-C at week 24 relative to placebo. The LS mean (SE) % change from baseline at week 24 was −48.8% for the alirocumab group (N=488), compared to 7.1% for the placebo group (N=244). The LS mean difference (SE) vs. placebo was −55.8% (2.1) (P<0.0001). Moreover, only 42% of of alirocumab patients required uptitration at Week 12 to the 150 mg Q2W dose.

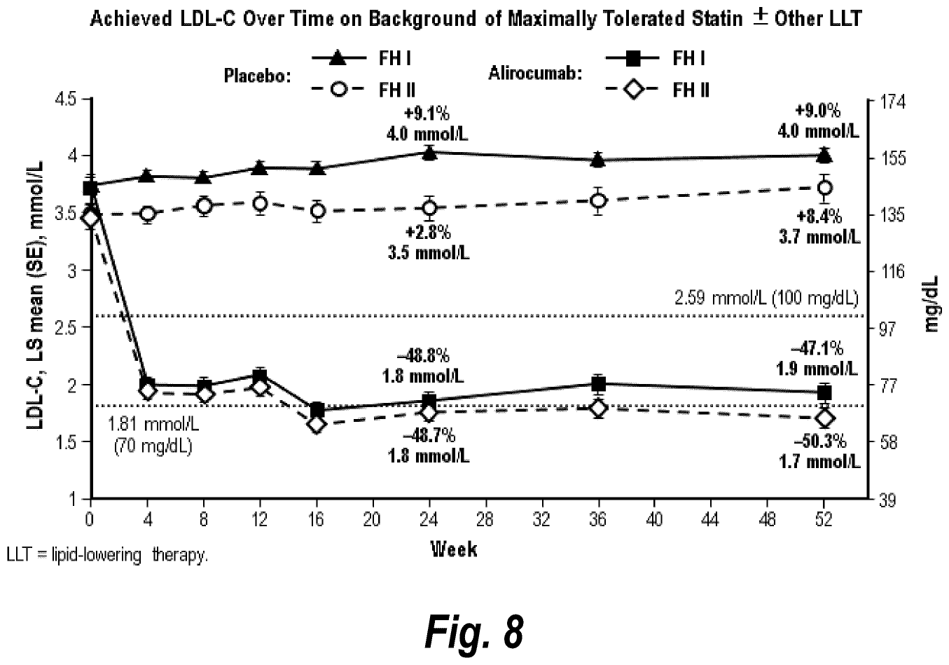

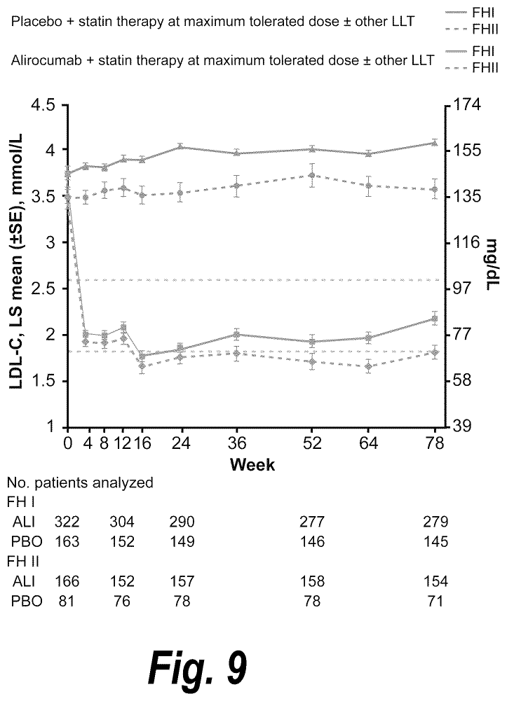

The LS mean (SE) calculated LDL-C values versus time for the ODYSSEY FH I and FH II studies are shown in FIG. 8. The values indicted on the graph are the LS mean % change from baseline to week 24 and week 52. FIG. 9 is a graph showing the LS mean (SE) calculated LDL-C values versus time for the ODYSSEY FH I and FH II studies. The values indicted below the graph are the numbers of patients analyzed at the various timepoints.

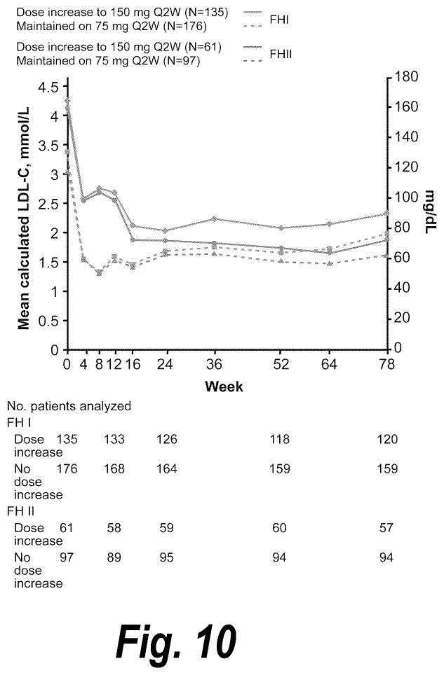

Among patients who received double-blind treatment for at least 12 weeks, 176/311 (56.6%) in FH I and 97/158 (61.4%) in FH II had LDL-C levels <1.8 mmol/L at week 8 and were maintained on alirocumab 75 mg Q2W. LDL-C levels were stable over time in these patients (FIG. 10). For patients in FH I who received dose increase to 150 mg Q2W, mean LDL-C levels were 2.7 mmol/L (104.3 mg/dL) at week 12 and 2.0 mmol/L (78.5 mg/dL) at week 24. Corresponding values in FH II were 2.6 mmol/L (98.6 mg/dL) at week 12 and 1.9 mmol/L (71.8 mg/dL) at week 24.

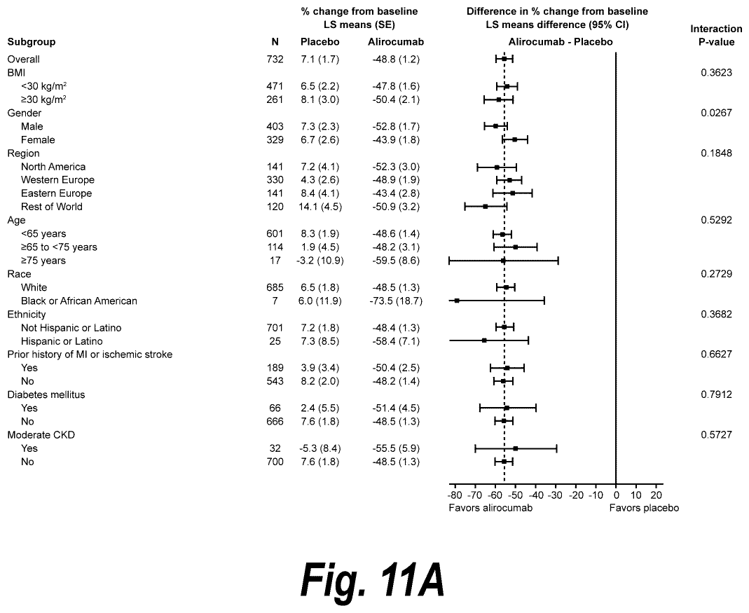

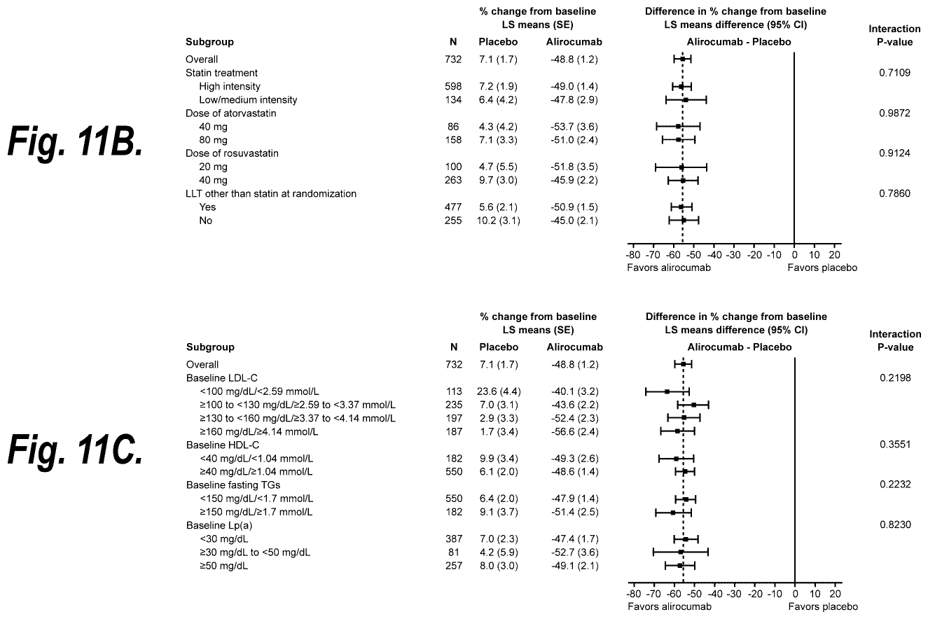

Subgroup analyses of the primary efficacy endpoint showed consistent reduction of calculated LDL-C across a range of demographic and baseline characteristics (FIGS. 11A-11C). The percentage reduction in LDL-C (alirocumab vs placebo) was 60.1% in males and 50.6% in females (pooled data from FH I and FH II), with a P-value for interaction of 0.0267. In the individual studies, LDL-C reductions (vs placebo) were 62.6% for males and 51.9% for females in FH I, and 53.5% for males and 49.2% for females in FH II.

A summary of interim safety data pooled from the FH I and FH II studies is set forth in Table 25A. All data was collected up to last patient visit at week 52. The percentage of patients who experienced TEAEs, serious AEs, and TEAEs leading to treatment discontinuation were comparable between treatment groups in the individual studies (Table 25B). A higher proportion of patients experienced injection site reactions in the alirocumab groups vs placebo in FH I (12.4% vs 11.0%) and FH II (11.4% vs 7.4%). Most of the injection site reactions were classified as mild in intensity. No injection site reaction led to study drug discontinuation. None of the reported neurological or allergic events (Table 3) were serious. Pruritus was reported in two (0.6%) and three (1.8%) alirocumab-treated patients in FH I and II, respectively, and one placebo-treated patient in each study (0.6% and 1.2%, respectively). Few neurocognitive events were reported with alirocumab (2 [0.6%] in FH I, none in FH II) or placebo (2 [1.2%] in FH I, 1 [1.2%] in FH II; Table 3). In FH I and FH II, respectively, 85.8% and 91.6% of alirocumab-treated patients (87.7% and 90.1% of placebo) received study treatment for ≥76 weeks.

TABLE 25A
Interim Safety Analysis (Pooled Data from FH I and FH II Studies)
% (N) of patients
All pts on background of max

| tolerated statin ± other | Alirocumab | | Placebo | |
|---|---|---|---|---|
| lipid-lowering therapy | (N = 489) | | (N = 244) | |
| TEAEs | 74.8% | (366) | 75.4% | (184) |
| Treatment-emergent SAEs | 10.0% | (49) | 9.0% | (22) |
| TEAEs leading to death | 0.8% | (4) | 0 | |
| TEAEs leading to | 3.1% | (15) | 3.7% | (9) |
| discontinuation | | | | |

Adverse Events of Interest

| | | | | |
|---|---|---|---|---|
| Adjudicated CV events | 1.6% | (8) | 1.2% | (3) |
| Injection-site reactions | 11.5% | (56) | 9.0% | (22) |
| Neurocognitive disorders | 0.2% | (1) | 1.2% | (3) |
| ALT >3 × ULN | 2.1% | (10/488) | 1.2% | (3/244) |
| Creatine kinase >3 × ULN | 3.5% | (17/483) | 6.2% | (15/243) |

Other Adverse Events

| | | | | |
|---|---|---|---|---|
| Nasopharyngitis | 10.2% | (50) | 11.1% | (27) |
| Influenza | 8.8% | (43) | 6.1% | (15) |
| Headache | 5.5% | (27) | 6.6% | (16) |

| | | |
|---|---|---|
| Back pain | 4.9% | 3.7% |
| Upper respiratory tract | 4.3% | 4.9% |
| infection | | |
| arthralgia | 3.9% | 4.9% |
| urinary tract infection | 3.9% | 2.5% |
| Diarrhoea | 3.7% | 2.5% |
| Myalgia | 3.5% | 4.9% |
| gastroenteritis | 3.3% | 3.3% |
| sinusitis | 3.3% | 2.9% |
| muscle spasms | 3.1% | 0.4% |
| dizziness | 2.9% | 3.7% |
| nausea | 2.5% | 3.7% |
| pain in extremities | 1.8% | 3.3% |
| fatigue | 3.1% | 2.5% |
| influenza like illness | 2.9% | 2.0% |
| bronchitis | 2.7% | 2.5% |
| abdominal pain | 2.5% | 1.6% |
| blood creatinine | 2.5% | 2.9% |
| phosphokinase cough | | |
| hypertension | 1.6% | 2.5% |
| cystitis | 1.2% | 1.6% |
| neck pain | 0.4% | 2.0% |

TABLE 25B
Final Safety Analysis (Pooled Data from FHI and FHII Studies)

| | FH I | | | | FH II | | | |
|---|---|---|---|---|---|---|---|---|
| | Alirocumab | | Placebo | | Alirocumab | | Placebo | |
| n (%) | (n = 322) | | (n = 163) | | (n = 167) | | (n = 81) | |
| TEAEs | 263 | (81.7) | 129 | (79.1) | 125 | (74.9) | 66 | (81.5) |
| Treatment-emergent SAEs | 44 | (13.7) | 22 | (13.5) | 15 | (9.0) | 8 | (9.9) |
| TEAEs leading to death^{a} | 6 | (1.9) | 0 | | 0 | | 0 | |
| TEAEs leading to treatment | 11 | (3.4) | 10 | (6.1) | 6 | (3.6) | 1 | (1.2) |
| discontinuation | | | | | | | | |

TEAEs occurring in ≥5% patients (in any group)

| Injection site reaction | 40 | (12.4) | 18 | (11.0) | 19 | (11.4) | 6 | (7.4) |
|---|---|---|---|---|---|---|---|---|
| Exact Fisher test p-value^{b} | 0.77 | | | | 0.38 | | | |
| Nasopharyngitis | 36 | (11.2) | 12 | (7.4) | 21 | (12.6) | 18 | (22.2) |
| Upper respiratory tract infection | 22 | (6.8) | 14 | (8.6) | 5 | (3.0) | 1 | (1.2) |
| Arthralgia | 20 | (6.2) | 9 | (5.5) | 8 | (4.8) | 7 | (8.6) |
| Influenza | 20 | (6.2) | 10 | (6.1) | 24 | (14.4) | 7 | (8.6) |
| Back pain | 18 | (5.6) | 7 | (4.3) | 12 | (7.2) | 6 | (7.4) |
| Sinusitis | 17 | (5.3) | 7 | (4.3) | 1 | (0.6) | 2 | (2.5) |
| Headache | 15 | (4.7) | 9 | (5.5) | 16 | (9.6) | 7 | (8.6) |
| Diarrhoea | 10 | (3.1) | 5 | (3.1) | 11 | (6.6) | 1 | (1.2) |
| Bronchitis | 10 | (3.1) | 9 | (5.5) | 4 | (2.4) | 1 | (1.2) |
| Dizziness | 7 | (2.2) | 6 | (3.7) | 8 | (4.8) | 5 | (6.2) |
| Myalgia | 6 | (1.9) | 11 | (6.7) | 10 | (6.0) | 5 | (6.2) |
| Influenza like illness | 6 | (1.9) | 1 | (0.6) | 9 | (5.4) | 5 | (6.2) |

Safety events of interest

| Positively adjudicated CV events | 8 | (2.5) | 3 | (1.8) | 2 | (1.2) | 1 | (1.2) |
|---|---|---|---|---|---|---|---|---|
| General allergic TEAEs^{c} | 28 | (8.7) | 16 | (9.8) | 19 | (11.4) | 5 | (6.2) |
| Neurological TEAEs^{c} | 12 | (3.7) | 7 | (4.3) | 7 | (4.2) | 2 | (2.5) |
| Neurocognitive disorders^{c} | 2 | (0.6) | 2 | (1.2) | 0 | | 1 | (1.2) |
| Development/worsening of | 6 | (1-9) | 4 | (2.5) | 4 | (2.4) | 2 | (2.5) |
| diabetes^{b} | | | | | | | | |
| Ophthalmologic disorders^{c} | 3 | (0.9) | 4 | (2.5) | 3 | (1.8) | 1 | (1.2) |
| Alanine aminotransferase >3 × | 5/322 | (1.6) | 2/163 | (1.2) | 6/166 | (3.6) | 1/81 | (1.2) |
| ULN | | | | | | | | |
| Creatine kinase >3 × | 13/318 | (4.1) | 10/163 | (6.1) | 8/165 | (4.8) | 6/80 | (7.5) |
| ULN | | | | | | | | |

### Example 4: A Randomized, Double-Blind, Placebo-Controlled, Parallel Group Study to Evaluate the Efficacy and Safety of Alirocumab in Patients with Heterozygous Familial Hypercholesterolemia and LDL-C Higher or Equal to 160 mg/dL with their Lipid-Modifying Therapy

### Introduction

This study included patients with heterozygous familial hypercholesterolemia (heFH) with or without a history of documented MI or ischemic stroke.

The objective of the present study was to assess the efficacy and safety of Alirocumab in patients with heFH whose LDL-C level was higher than or equal to 160 mg/dL (4.14 mmol/L) on maximally tolerated statin therapy with or without additional LMT.

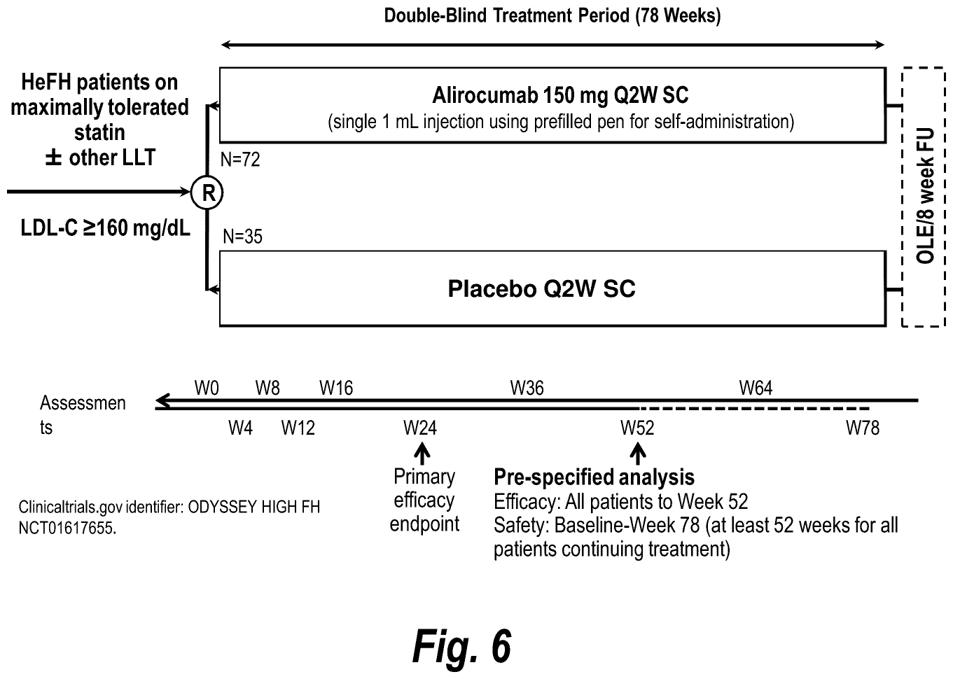

This specific study (FIG. 6) was undertaken to demonstrate in heFH patients, with LDL-C higher or equal to 160 mg/dL, that Alirocumab 150 mg Q2W as add-on therapy to statin+/−other LMT causes a statistically significant and clinically meaningful reduction in LDL-C. This population with such a high LDL-C level despite an optimized LMT represents a highest risk group with a well-identified unmet medical need that may be addressed by adding Alirocumab to their LDL-C lowering therapies.

### Study Objectives

The primary objective of the study was to demonstrate the reduction of LDL-C by Alirocumab as add-on therapy to stable maximally tolerated daily statin therapy with or without other LMT in comparison with placebo after 24 weeks of treatment in patients with heterozygous familial hypercholesterolemia (heFH) and LDL-C higher than or equal to 160 mg/dL (4.14 mmol/L).

The secondary objectives were: 1) to evaluate the effect of Alirocumab in comparison with placebo on LDL-C after 12 weeks of treatment; 2) to evaluate the effect of Alirocumab on other lipid parameters (i.e., Apo B, non-HDL-C, total-C, Lp (a), HDL-C, TG, and Apo A-1 levels); 3) to evaluate the long-term effect of Alirocumab on LDL-C; 4) to evaluate the safety and tolerability of Alirocumab; 5) to evaluate the development of anti-Alirocumab antibodies.

### Study Design

This was a randomized, double-blind, placebo-controlled, parallel-group, unbalanced (2:1, Alirocumab: placebo), multi-center, multi-national study to assess the efficacy and the safety of Alirocumab in patients with heterozygous familial hypercholesterolemia (heFH) and LDL-C higher or equal to 160 mg/dL with or without their LMT (i.e., stable maximally tolerated daily statin therapy+/−other LMT). Randomization was stratified according to prior history of myocardial infarction (MI) or ischemic stroke [Yes/No], and statin treatment (atorvastatin 40 to 80 mg daily or rosuvastatin 20 to 40 mg daily vs. simvastatin whatever the daily dose, atorvastatin below 40 mg daily or rosuvastatin below 20 mg daily). After randomization, patients received double-blind study treatment (either Alirocumab or placebo) every 2 weeks over a period of 78 weeks on top of stable maximally tolerated daily statin therapy+/−other LMT.

After completion of the 18-month double-blind treatment period, all patients who successfully completed the ODYSSEY High FH study had the opportunity to participate in an open-label extension study. Consequently all patients will receive Alirocumab at entry in the open-label extension study regardless the study treatment they received during the 18-month double-blind treatment period.

The study consisted of 3 periods: screening, double-blind treatment, and follow up.

The screening period was up to 3 weeks in duration including an intermediate visit during which the patient (or another designated person such as spouse, relative, etc.) was trained to self-inject/inject with placebo for Alirocumab. Eligibility assessments were performed to permit the randomization of the patients into the study.

The double-blind treatment period was a randomized, double-blind study treatment period of 18 months. The first injection during the double-blind period was done at the site on the day of randomization (Week 0 [D1]-V3) and as soon as possible after the call to IVRS/IWRS for randomization into the study. The subsequent injections were done by the patient (self-injection) or another designated person (such as spouse, relative, etc.) at a patient-preferred location (home . . . ). Patients randomized to Alirocumab received a dose of 150 mg of the IMP from randomization (V3) up to Week 76 (i.e., Weeks 0, 2, 4, 6, 8 . . . to 76).

The follow-up period (if applicable) was a period of 8 weeks after the end of the DBTP for patients not consenting to participate in the open-label extension study or if prematurely discontinuing study treatment.

The laboratory measurement of lipid parameters were performed by a central laboratory (central lab) during the study.

Patients who achieved 2 consecutive calculated LDL-C levels <25 mg/dL (0.65 mmol/L) during the study were monitored and managed.

Statin and other LMT (if applicable) should have been stable (including dose) during the first 24 weeks of the DBTP barring exceptional circumstances whereby overriding concerns (including but not limited to TG alert posted by the central lab) warrant such changes, as per the Investigator's judgment. From Week 24 onwards, background LMT was modified only under certain conditions as described below.

Patients should have been on a stable diet (NCEP-ATPIII TLC diet or equivalent) throughout the entire study duration from screening, as described above in Example 2 (see Table 1). The dietician or site staff with appropriate training reviewed the patient's diet at the screening visit and periodically throughout the study.

The study duration included a screening period of up to 3 weeks, a 78-week DBTP for efficacy and safety assessment, and an 8-week post-treatment follow-up period after the last visit of the DBTP for patients not consenting to participate in the open-label extension study or if prematurely discontinuing study treatment. Thus, the maximum study duration per patient was about 89 weeks (i.e., 20 months) (up to 3 weeks screening+78 weeks double-blind treatment+8 weeks follow-up). The end of the study per patient was the last protocol planned visit or the resolution/stabilization of all SAEs, and AESI, whichever came last.

### Selection of Patients

The inclusion criteria were: 1) patients with heterozygous familial hypercholesterolemia (heFH)* who were not adequately controlled with a maximally tolerated daily dose of statin,** with or without other lipid-modifying therapy (LMT) at stable dose prior to the screening visit (Week-3). *Diagnosis of heFH must have been made either by genotyping or by clinical criteria. For those patients not genotyped, the clinical diagnosis may have been based on either the Simon Broome criteria with a criteria for definite FH or the WHO/Dutch Lipid Network criteria with a score >8 points. See criteria described above in Example 2.

Definition of maximally tolerated dose: any of the following were acceptable): 1) rosuvastatin 20 mg or 40 mg daily; 2) atorvastatin 40 mg or 80 mg daily; 3) simvastatin 80 mg daily (if already on this dose for >1 year—see exclusion criterion E 06); or 4) patients not able to be on any of the above statin doses, should have been treated with the dose of daily atorvastatin, rosuvastain or simvastatin that was considered appropriate for the patient as per the investigator's judgment or concerns. Some examples of acceptable reasons for a patient taking a lower statin dose included, but were not limited to: adverse effects on higher doses, advanced age, low body mass index, regional practices, local prescribing information, concomitant meds, co-morbid conditions such as impaired glucose tolerance/impaired fasting glucose.

Patients who met all the above inclusion criteria were screened for the following exclusion criteria, which are sorted and numbered in the following 3 subsections: exclusion criteria related to study methodology, exclusion criteria related to the background therapies, and exclusion criteria related to Alirocumab.

Exclusion criteria related to study methodology were: 1) patient without diagnosis of heFH made either by genotyping or by clinical criteria; 2) LDL-C<160 mg/dL (<4.14 mmol/L) at the screening visit (Week-3); 3) not on a stable dose of LMT (including statin) for at least 4 weeks and/or fenofibrate for at least 6 weeks as applicable, prior to the screening visit (Week-3) or from screening to randomization; 4) currently taking a statin other than simvastatin, atorvastatin, or rosuvastatin; 5) simvastatin, atorvastatin, or rosuvastatin is not taken daily or not taken at a registered dose; 6) daily doses above atorvastatin 80 mg, rosuvastatin 40 mg or simvastatin 40 mg, (except for patients on simvastatin 80 mg for more than one year, who are eligible); 7) use of fibrates, other than fenofibrate within 6 weeks of the screening visit (Week-3) or between screening and randomization visits; 8) use of nutraceutical products or over-the-counter therapies that may affect lipids which have not been at a stable dose/amount for at least 4 weeks prior to the screening visit (Week-3) or between screening and randomization visits; 9) use of red yeast rice products within 4 weeks of the screening visit (Week-3) or between screening and randomization visits; 10) patient who has received plasmapheresis treatment within 2 months prior to the screening visit (Week-3), or has plans to receive it during the study; 11) recent (within 3 months prior to the screening visit [Week-3] or between screening and randomization visits) MI, unstable angina leading to hospitalization, percutaneous coronary intervention (PCI), coronary artery bypass graft surgery (CABG), uncontrolled cardiac arrhythmia, stroke, transient ischemic attack (TIA), carotid revascularization, endovascular procedure or surgical intervention for peripheral vascular disease; 12) planned to undergo scheduled PCI, CABG, carotid, or peripheral revascularization during the study; 13) systolic blood pressure >160 mmHg or diastolic blood pressure >100 mmHg at screening visit or randomization visit; 14) history of New York Heart Association (NYHA) Class III or IV heart failure within the past 12 months; 15) known history of a hemorrhagic stroke; 16) age <18 years or legal age of majority at the screening visit (Week-3), whichever is greater; 17) patients not previously instructed on a cholesterol-lowering diet prior to the screening visit (Week-3); 18) newly diagnosed (within 3 calendar months prior to randomization visit [Week 0]) or poorly controlled (HbA1c>9% at the screening visit [Week-3]) diabetes; 19) presence of any clinically significant uncontrolled endocrine disease known to influence serum lipids or lipoproteins. Note: patients on thyroid replacement therapy can be included if the dosage has been stable for at least 12 weeks prior to screening and between screening and randomization visits, and TSH level is within the normal range of the Central Laboratory at the screening visit; 20) history of bariatric surgery within 12 months prior to the screening visit (Week-3); 21) unstable weight defined by a variation >5 kg within 2 months prior to the screening visit (Week-3); 22) known history of homozygous FH; 23) known history of loss of function of PCSK9 (i.e., genetic mutation or sequence variation); 24) use of systemic corticosteroids, unless used as replacement therapy for pituitary/adrenal disease with a stable regimen for at least 6 weeks prior to randomization visit (Week 0). Note: topical, intra-articular, nasal, inhaled and ophthalmic steroid therapies are not considered as “systemic” and are allowed; 25) use of continuous estrogen or testosterone hormone replacement therapy unless the regimen has been stable in the past 6 weeks prior to the Screening visit (Week-2) and no plans to change the regimen during the study; 26) history of cancer within the past 5 years, except for adequately treated basal cell skin cancer, squamous cell skin cancer or in situ cervical cancer; 27) known history of positive HIV test; 28) patient who has taken any investigational drugs other than the Alirocumab training placebo kits within 1 month or 5 half lives, whichever is longer; 29) patient who has been previously treated with at least one dose of Alirocumab or any other anti-PCSK9 monoclonal antibody in other clinical trials; 30) patient who withdraws consent during the screening period (patient who is not willing to continue or fails to return); 31) conditions/situations such as any clinically significant abnormality identified at the time of screening that, in the judgment of the Investigator or any sub-Investigator, would preclude safe completion of the study or constrain endpoints assessment; eg, major systemic diseases, patients with short life expectancy considered by the Investigator or any sub-Investigator as inappropriate for this study for any reason, e.g.,: a) deemed unable to meet specific protocol requirements, such as scheduled visits; b) deemed unable to administer or tolerate long-term injections as per the patient or the Investigator; c) investigator or any sub-Investigator, pharmacist, study coordinator, other study staff or relative thereof directly involved in the conduct of the protocol, etc.; d) presence of any other conditions (e.g., geographic or social . . . ) actual or anticipated, that the Investigator feels would restrict or limit the patient's participation for the duration of the study; 32) laboratory findings during screening period (not including randomization Week 0 labs): a) positive test for Hepatitis B surface antigen or Hepatitis C antibody; b) positive serum beta-hCG or urine pregnancy (including Week 0) in women of childbearing potential; c) triglycerides >400 mg/dL (>4.52 mmol/L) (1 repeat lab is allowed); d) eGFR<30 mL/min/1.73 m2 according to 4-variable MDRD Study equation (calculated by central lab); e) ALT or AST>3×ULN (1 repeat lab is allowed); f) CPK>3×ULN (1 repeat lab is allowed); g) TSH<lower limit of normal (LLN) or >upper limit of normal (ULN) (1 repeat lab is allowed).

Exclusion criteria related to the background therapies were: 1) all contraindications to the background therapies or warnings/precautions of use (when appropriate) as displayed in the respective National Product Labeling.

Exclusion criteria related to Alirocumab were: 1) known hypersensitivity to monoclonal antibody or any component of the drug product; 2) pregnant or breast-feeding women; and 3) women of childbearing potential not protected by highly-effective method(s) of birth control (as defined in the informed consent form and/or in a local protocol addendum) and/or who are unwilling or unable to be tested for pregnancy. Note: Women of childbearing potential must have had a confirmed negative pregnancy test at screening and randomization visits. They must have used an effective contraceptive method throughout the entire duration of the study treatment, and for 10 weeks after the last intake of IMP, and agreed to repeat urine pregnancy test at designated visits. The applied methods of contraception had to meet the criteria for a highly effective method of birth control according to the “Note for guidance on non-clinical safety studies for the conduct of human clinical trials and marketing authorization for pharmaceuticals (CPMP/ICH/286/95)”. Postmenopausal women must have been amenorrheic for at least 12 months.

### Study Treatments

Sterile Alirocumab drug product was supplied at a concentration of 150 mg/mL in histidine, pH 6.0, polysorbate 20, and sucrose. Drug product was supplied as 1 mL volume in an auto-injector.

Sterile placebo for Alirocumab was prepared in the same formulation as Alirocumab without the addition of protein as 1 mL volume in an auto-injector.

During the double-blind treatment period, Alirocumab or placebo was administered subcutaneously every 2 weeks, starting at Week 0 continuing up to the last injection (Week 76) 2 weeks before the end of the double blind treatment period. If the injection was scheduled to take place on the same date as the site visit, then the IMP should have been administered after the blood sampling had been completed.

IMP should ideally have been administered every 2 weeks subcutaneously at approximately the same time of the day; however it was acceptable to have a window period of ±3 days. The time of the day was based on patient's preference.

The following classes of drugs were identified as non-investigational medicinal products (NIMP) because the medication was either a background therapy or a potential rescue medication: statins (rosuvastatin, atorvastatin, simvastatin); cholesterol absorption inhibitors (ezetimibe); bile acid-binding sequestrants (such as cholestyramine, colestipol, colesevelam); nicotinic acid; fenofibrate; omega-3 fatty acids (≥1000 mg daily).

Patients who achieved 2 consecutive calculated LDL-C<25 mg/dL (0.65 mmol/L) were monitored.

Patients who had titers at or above 240 for anti-Alirocumab antibodies at follow-up visit had additional antibody sample(s) at 6 to 12 months after the last dose, and thereafter about every 3 to 6 months until titer returned below 240.

Patients were randomized to receive either placebo or Alirocumab during the double-blind study treatment period using a ratio 1:2, with permuted-block randomization. Randomization was stratified according to prior history of myocardial infarction (MI) or ischemic stroke [Yes/No], and statin treatment (atorvastatin 40 to 80 mg daily or rosuvastatin 20 to 40 mg daily vs. simvastatin whatever the daily dose, atorvastatin below 40 mg daily or rosuvastatin below 20 mg daily).

A concomitant medication was any treatment received by the patient concomitantly to the study (until follow-up visit). Concomitant medications were to be kept to a minimum during the study. However, if these were considered necessary for the patient's welfare and were unlikely to interfere with the IMP, they couuld be given at the discretion of the Investigator, with a stable dose (when possible). Besides the specific information related to concomitant medications provided in this section, any other concomitant medication(s) will be allowed. If the patient had an LDL-C > or equal 160 mg/dL (4.14 mmol/L) at the screening visit (Week-3) and was treated with a statin only, i.e. without additional LMT, the investigator was to report the reason for the patient not being on a second LMT. For background LMT, including statins, sites must have followed the national product label for the safety monitoring and management of patients.

Nutraceutical products or over-the-counter therapies that may affect lipids were allowed only if they had been used at a stable dose for at least 4 weeks prior to screening visit, during the screening period and maintained during the first 24 weeks of the double-blind treatment period. After the Week 24 visit, modification to these nutraceutical products or over-the-counter therapies was allowed but in general should have been avoided. Examples of such nutraceutical products or over-the-counter therapies included omega-3 fatty acids at doses <1000 mg, plant stanols such as found in Benecol, flax seed oil, and psyllium.

Patients must have been on stable maximally tolerated daily registered doses of statins with other LMT for at least 4 weeks (6 weeks for fenofibrate) before screening visit. During the study, the patients should have stayed on these stable maximally tolerated registered daily doses of statins with other LMT. Lipid profile values from samples obtained after randomization were blinded. Nevertheless, sites were made aware of triglyceride alert, as well as rescue threshold of LDL-C value in order to make decisions on the patient's background LMT. From the screening visit (Week-3) until Week 24 of the double-blind treatment period, the background LMT should not have been changed. No dose adjustment, discontinuation or initiation of other statins or other LMT should have taken place during this time, barring exceptional circumstances whereby overriding concerns (including but not limited to triglyceride alert posted by the central lab) warranted such changes, as per the investigator's judgment.

For a triglyceride alert (TG≥500 mg/dL (5.65 mmol/L)) that was confirmed by repeat testing, the investigator was to perform investigations, manage the patient, and modify the background LMT as per his/her medical judgment.

For a rescue notification of LDL-C at the Week 24 visit and later, i.e., LDL-C increase >25% as compared to randomization visit LDL-C on two consecutive occasions, the investigator should have ensured that no reasonable explanation existed for insufficient LDL-C control (such as an alternative medical cause like corticosteroid use, etc.) and in particular that: compliance with diet was appropriate, compliance with background LMT was appropriate, and study treatment was given as planned. If any of the above could have reasonably explained the insufficient LDL-C control, the investigator should have undertaken appropriate action, i.e. stressed the absolute need to be compliant with treatment, if needed organized a specific interview with a qualified nutrition professional and stressed the absolute need to be compliant with diet, and performed a blinded LDL-C assessment within 1 to 2 months. If none of the above mentioned reasons could be found, or if appropriate action failed to decrease LDL-C under the alert value, rescue medication may have been introduced. The effectiveness of any such changes were made based on the absence of rescue notification of LDL-C sent on the blinded lipid testing at the next scheduled lab draw.

If no reason for LDL-C above the threshold value could be found, or if appropriate action failed to decrease LDL-C below the threshold value, rescue medication may have been introduced. The effectiveness of any such changes would be made based on lack of rescue threshold from blinded lipid testing at the next routinely scheduled lab draw. Patients per protocol already received a maximum tolerated dose of statin, so statin up-titration or switch was not an option. For further LDL-C lowering, the investigator may have considered: a cholesterol absorption inhibitor (ezetimibe), or a bile acid-binding sequestrant (the resins cholestyramine and colestipol, or colesevelam, a nonabsorbable polymer). The following lipid modifying agents may have also been considered: fibrate (Note: Caution should be exercised when combining fibrates with other cholesterol-lowering medications such as statins because of the risk of myopathy. When a fibrate is combined with a statin, fenofibrate is the fibrate of choice because it does not affect statin glucuronidation.); the only fibrate allowed per protocol was fenofibrate; nicotinic acid (niacin) (Note: Niacin raises blood glucose but has been shown to be effective in modifying lipid disorders in people with diabetes if glucose control is maintained).

In summary, background LMT should not have been modified from screening to the follow up visit. However, up to Week 24, if a confirmed TG alert was reached or if there was an overwhelming clinical concern (at the discretion of the Investigator) then modification of the background LMT was allowed. From Week 24 onwards, if a confirmed TG alert was reached, or if a rescue threshold for LDL-C was attained (and no other reasonable explanation exists), or if there was an overwhelming clinical concern (at the discretion of the Investigator) then modification of the background LMT was allowed.

Women of childbearing potential were required to take an effective contraceptive method throughout the study treatment and for 10 weeks after the last IMP injection (i.e., Follow-up visit).

Forbidden concomitant medications from the initial screening visit until the follow-up visit included the following: statins other than simvastatin, atorvastatin and rosuvastatin; fibrates, other than fenofibrate; and red yeast rice products.

### Study Endpoints

The primary efficacy endpoint was the percent change in calculated LDL-C from baseline to Week 24, which was defined as: 100× (calculated LDL-C value at Week 24−calculated LDL-C value at baseline)/calculated LDL-C value at baseline. The baseline calculated LDL-C value was the last LDL-C level obtained before the first double-blind IMP injection. The calculated LDL-C at Week 24 was the LDL-C level obtained within the Week 24 analysis window and during the main efficacy period. The main efficacy period was defined as the time from the first double-blind IMP injection up to 21 days after the last double-blind IMP injection or up to the upper limit of the Week 24 analysis window, whichever came first. All calculated LDL-C values (scheduled or unscheduled, fasting or not fasting) may be used to provide a value for the primary efficacy endpoint if appropriate according to above definition.

The key secondary efficacy endpoints were: 1) The percent change in calculated LDL-C from baseline to Week 12: similar definition and rules as for primary efficacy endpoint, except that the calculated LDL-C at Week 12 was the LDL-C level obtained within the Week 12 analysis window and during the 12-week efficacy period. The 12-week efficacy period was defined as the time from the first double-blind IMP injection up to the Visit 6 re-supply IVRS contact or up to 21 days after the last double-blind IMP injection, whichever came first. Blood sampling collected the day of the Visit 6 re-supply IVRS contact was considered as before titration; 2) the percent change in Apo B from baseline to Week 24. Same definition and rules as for the primary endpoint; 3) the percent change in non-HDL-C from baseline to Week 24. Same definition and rules as for the primary endpoint; 4) the percent change in total-C from baseline to Week 24. Same definition and rules as for the primary endpoint; 5) the percent change in Apo B from baseline to Week 12. Same definition and rules as for the percent change in calculated LDL-C from baseline to Week 12; 6) the percent change in non-HDL-C from baseline to Week 12. Same definition and rules as for the percent change in calculated LDL-C from baseline to Week 12; 7) the percent change in total-C from baseline to Week 12. Same definition and rules as for the percent change in calculated LDL-C from baseline to Week 12; 8) the percent change in calculated LDL-C from baseline to Week 52. Definitions and rules were similar to the ones used for the primary endpoint replacing Week 24 by Week 52. The 52-week efficacy period was defined as the time from the first double-blind IMP up to 21 days after the last double-blind IMP injection, or up to the upper limit of the Week 52 analysis window whichever came first; 9) the proportion of patients reaching LDL-C goal at Week 24, i.e. LDL-C<70 mg/dL (1.81 mmol/L) in case of prior CVD or <100 mg/dL (2.59 mmol/L) for patients without CVD, defined as: (number of patients whose calculated LDL-C value at Week 24 reach LDL-C goal/number of patients in the mITT population)*100, using definition and rules used for the primary endpoint; 10) the percent change in Lp(a) from baseline to Week 24. Same definition and rules as for the primary endpoint; 11) the percent change in HDL-C from baseline to Week 24. Same definition and rules as for the primary endpoint; 12) the percent change in HDL-C from baseline to Week 12. Same definition and rules as for the percent change in calculated LDL-C from baseline to Week 12; 13) the percent change in Lp(a) from baseline to Week 12. Same definition and rules as for the percent change in calculated LDL-C from baseline to Week 12; 14) the percent change in fasting TG from baseline to Week 24. Same definition and rules as for the primary endpoint; 15) the percent change in fasting TG from baseline to Week 12. Same definition and rules as for the percent change in calculated LDL-C from baseline to Week 12; 16) the percent change in Apo A-1 from baseline to Week 24. Same definition and rules as for the primary endpoint; 17) the percent change in Apo A-1 from baseline to Week 12. Same definition and rules as for the percent change in calculated LDL-C from baseline to Week 12.

Other secondary efficacy endpoints were: 1) the percent change in calculated LDL-C from baseline to Week 78: Definitions and rules were similar to the ones used for the primary endpoint replacing Week 24 by Week 78; 2) the proportion of patients reaching LDL-C goal at Weeks 12, 52 and 78, i.e., LDL-C<70 mg/dL (1.81 mmol/L) in case of prior CVD or <100 mg/dL (2.59 mmol/L) for patients without prior CVD; 3) the proportion of patients reaching LDL C<100 mg/dL (2.59 mmol/L) at Week 24; 4) the proportion of patients reaching LDL-C<100 mg/dL (2.59 mmol/L) at Week 12; 5) the proportion of patients reaching LDL-C<70 mg/dL (1.81 mmol/L) at Week 24; 6) the proportion of patients reaching LDL-C<70 mg/dL (1.81 mmol/L) at Week 12; 7) the absolute change in calculated LDL-C (mg/dL and mmol/L) from baseline to Weeks 12, 24, 52 and 78; 8) the percent change in Apo B, non-HDL-C, total-C, Lp (a), HDL-C, fasting TG, and Apo A-1 from baseline to Week 52 and 78; 9) the change in ratio Apo B/Apo A-1 from baseline to Weeks 12, 24, 52 and 78; 10) the proportion of patients with Apo B<80 mg/dL (0.8 g/L) at Weeks 12, 24, 52 and 78; 11) the proportion of patients with non-HDL-C<100 mg/dL at Weeks 12, 24, 52 and 78; 12) the proportion of patients with calculated LDL-C<70 mg/dL (1.81 mmol/L) and/or ≥50% reduction in calculated LDL-C (if calculated LDL-C≥70 mg/dL [1.81 mmol/L]) at Weeks 12, 24, 52 and 78.

Total-C, HDL-C, TG, Apo B, Apo A-1, and Lp (a) were directly measured by the Central Laboratory. LDL-C was calculated using the Friedewald formula at all visits (except Week −1 and Follow Up visit). If TG values exceeded 400 mg/dL (4.52 mmol/L) then the central lab reflexively measured (via the beta quantification method) the LDL-C rather than calculating it. Non-HDL-C was calculated by subtracting HDL-C from the total-C. Ratio Apo B/Apo A-1 was calculated.

The clinical laboratory data consisted of urinalysis, hematology (red blood cell count, hemoglobin, red blood cell distribution width (RDW), reticulocyte count, hematocrit, platelets, white blood cell count with differential blood count), standard chemistry (glucose, sodium, potassium, chloride, bicarbonate, calcium, phosphorous, urea nitrogen, creatinine, uric acid, total protein, LDH, albumin, γ Glutamyl Transferase [γGT]), Hepatitis C antibody, liver panel (ALT, AST, ALP, and total bilirubin), and CPK.

Vital signs included: heart rate, systolic and diastolic blood pressure in sitting position.

Other endpoints included: anti-Alirocumab antibody assessments, hs-CRP, HbA_{1c}, EQ-5D Questionnaire, and pharmacogenetic samples.

Anti-Alirocumab antibodies included the antibody status (positive/negative) and antibody titers. Serum samples for anti-Alirocumab antibody determination were drawn periodically throughout the study. The first scheduled sample at randomization visit was obtained before IMP injection (predose). Patients who had titers at or above 240 for anti-Alirocumab antibodies at the follow-up visit had additional antibody sample(s) at 6 to 12 months after the last dose, and thereafter about every 3 to 6 months until titer returned below 240. Anti-Alirocumab antibody samples were analyzed using a validated non-quantitative, titer-based bridging immunoassay. It involved an initial screen, a confirmation assay based on drug specificity, and a measurement of the titer of anti-Alirocumab antibodies in the sample. The lower limit of detection was approximately 1.5 ng/mL. Samples that were positive in the ADA assay were assessed for neutralizing antibodies using a validated, non-quantitative, competitive ligand binding assay. The lower limit of detection based on a monoclonal positive control neutralizing antibody was 390 ng/mL.

The percent change in hs-CRP from baseline to Week 24, 52 and 78.

The absolute change in HbA1c (%) from baseline to Week 24, 52 and 78.

EQ-5D is a standardized measure of health status developed by the EuroQol Group in order to provide a simple, generic measure of health for clinical and economic appraisal. The EQ-5D as a measure of health-related quality of life defines health in terms of 5 dimensions: mobility, self-care, usual activities, pain/discomfort, anxiety/depression. Each dimension can take one of three responses (3 ordinal levels of severity): ‘no problem’ (1), “some problems” (2), “severe problems” (3). Overall health state was defined as a 5-digit number. Health states defined by the 5-dimensional classification can be converted into corresponding index scores that quantify health status, where 0 represents ‘death’ and 1 represents “perfect health”.

### Study Procedures

For all visits after Day 1/Week 0 (randomization visit), a timeframe of a certain number of days was allowed. The window period for visits at Weeks 12 and 24 was ±3 days, at Week 52 and 78 was ±5 days, and for all other site visits it was ±7 days during the double-blind treatment period, and follow-up period. A window period of +3 days was allowed for the randomization visit (Day1/Week 0) and ±7 days for the injection training visit at screening (Week-1).

The blood sampling for determination of lipid parameters (i.e. total-C, LDL-C, HDL-C, TG, non-HDL-C, Apo B, Apo A-1, ratio Apo B/Apo A-1, Lp [a]) was to be performed in the morning, in fasting condition (i.e. overnight, at least 10-12 hours fast and refrain from smoking) for all site visits throughout the study. Alcohol consumption within 48 hours and intense physical exercise within 24 hours preceding the blood sampling were discouraged. Note: if the patient was not in fasting conditions, the blood sample was not be collected and a new appointment was given the day after (or as close as possible to this date) to the patient with instruction to be fasted (see above conditions).

Only patients who met the inclusion criteria were screened. The screening period took place up to 3 weeks or 21 days (and as short as possible, upon receipt of laboratory eligibility criteria) prior to randomization/Day 1 visit. The first screening visit (Week-3) took place from 21 to 8 days before the randomization visit. If it was planned to have another designated person administer the injections to the patient during the study, then this person should have been present at the injection training visit (Week −1).

The following visits were scheduled: Screening Visit (Visit1/Week −3/Day −21 up to −8); Screening (Visit2/Week −1/Day −7±7); Randomization visit (Visit 3/Week 0/Day 1+3); Visit 4/Week 4/Day 29±7); Visit 5/Week 8/Day 57±7); Visit 6/Week 12/Day 85±3; Visit 7/Week 16/Day 113±7): Visit 8/Week 24/Day 169±3; Visit 9/Week 36/Day 253±7; Visit 10/Week 52/Month 12/Day 365±5; Visit 11/Week 64/Day 449±7; Visit 12/Week 78/Month 18/Day 547±5 (end of treatment visit); and Visit 13/Week 86/Day 603±7 (follow up visit).

### Safety

Monitored safety events were the occurrence of treatment emergent adverse events (TEAEs) reported by the patient or noted by the investigator, serious adverse events (SAEs), TEAEs leading to treatment discontinuation, AEs of special interest (local Injection site reactions, allergic events, selected neurological events and cardiovascular events with adjudication result), occurrence of PCSA (potentially clinically significant abnormalities) in laboratory parameters, specific analysis for diabetes or impaired glucose control and patients with 2 consecutives LDL-C<25 mg/dL.

### Statistical Methods

### Sample Size Determination:

A total sample size of 45 patients (30 in alirocumab and 15 in placebo) had 95% power to detect a difference in mean percent change in LDL-C of 30% with a 0.05 two-sided significance level and assuming a common standard deviation of 25%, and all these 45 patients having an evaluable primary endpoint. A final total sample size of 105 patients with a randomization ratio 2:1 (alirocumab 70: placebo 35) has been selected in order to provide at least 50 patients exposed to alirocumab for 12 months at the first step analysis and assuming a drop-out rate of 10% over the first 3-month period and a drop-out rate of 20% over the 3-12-month period.

### Timing of Analyses:

The first step analysis included efficacy endpoints up to Week 52 (final efficacy analysis) and interim safety analysis, which was performed on all safety data up to the common study cut-off date (last patient Week 52 visit). Analysis of lipid data beyond Week 52 was descriptive. Results of the first step analysis are presented herein.

Second step (final) analysis will be conducted at the end of the study and will consist in the final analysis of efficacy endpoints up to Week 78 and final safety analysis.

### Analysis Populations:

The primary efficacy analysis population was the intent-to-treat (ITT) population, defined as all randomized patients who had an evaluable primary efficacy endpoint, that is, those with an available baseline calculated LDL-C value, and at least one available calculated LDL-C value within one of the analysis windows up to Week 24 (including all calculated LDL-C on-treatment and off-treatment).

The secondary efficacy analysis population was the modified intent-to-treat (mITT) population, defined as all randomized patients who took at least one dose or part of a dose of the double-blind investigational medicinal product (IMP) and who had an available calculated LDL-C value at baseline and at least one within one of the analysis windows up to Week 24 during the efficacy treatment period. The efficacy treatment period was defined as the time from the first double-blind IMP administration up to 21 days after the last double-blind injection.

The safety population included all randomized patients who received at least one dose or part of a dose of the double-blind IMP.

### Efficacy Analyses:

Primary analyses of efficacy endpoints were conducted using an ITT approach (based on the ITT population defined above), including all lipid data, regardless of whether the patient was continuing therapy or not. This corresponds to ITT estimands, defined for primary and key secondary endpoints. In addition, analyses were also conducted using an on-treatment approach (based on the mITT population defined above), including lipid data collected during the efficacy treatment period. This corresponds to on-treatment estimands of key secondary endpoints.

The ITT approach analyzed all patients, irrespective of their adherence to the treatment; it assessed the benefit of the treatment strategy and reflected as much as possible the effect in a population of patients. The on-treatment approach analyzed the effect of treatment, restricted to the period during which patients actually received the treatment. It assessed the benefit that a treatment would achieve in patients adherent to treatment up to the considered time point.

Efficacy analyses were performed according to treatment as-randomized.

All measurements, scheduled or unscheduled, fasting or not fasting, were assigned to analysis windows in order to provide an assessment for Week 4 to Week 78 time points.

With regards to the primary efficacy analysis (ITT approach), the percent change in calculated LDL-C from baseline to Week 24 was analyzed using a mixed-effect model with repeated measures (MMRM) approach. All post-baseline data available from Week 4 to Week 52 analysis windows were used and missing data were accounted for by the MMRM. The model included the fixed categorical effects of treatment group (placebo versus alirocumab), randomization strata (as per IVRS), time point (Week 4 to Week 52), treatment-by-time point interaction and strata-by-time point interaction, as well as, the continuous fixed covariates of baseline LDL-C value and baseline value-by-time-point interaction. This model provided baseline adjusted least-squares means (LSmeans) estimates at Week 24 for both treatment groups with their corresponding 95% confidence interval. To compare the alirocumab to the placebo group, an appropriate statement was used to test the differences of these estimates at the 5% alpha level.

A hierarchical procedure has been defined to test key secondary endpoints while controlling for multiplicity (using above order of key secondary endpoints). The first key secondary endpoint was the percent change in calculated LDL-C from baseline to Week 24 using an on-treatment approach.

Continuous secondary variables anticipated to have a normal distribution (i.e., lipids other than TG and Lp(a)) were analyzed using the same MMRM model as for the primary endpoint. Continuous endpoints anticipated to have a non-normal distribution (i.e., TG and Lp(a)) were analyzed using multiple imputation approach for handling of missing values followed by robust regression model with endpoint of interest as response variable using M-estimation (using SAS ROBUSTREG procedure) with treatment group, randomization strata (as per IVRS) and corresponding baseline value(s) as effects to compare treatment effects. Combined estimate for mean in both treatment groups, as well as the differences of these estimates, with their corresponding SEs, 95% CIs and p-value were provided (through SAS MIANALYZE procedure).

Binary secondary efficacy endpoints were analyzed using multiple imputation approach for handling of missing values followed by stratified logistic regression with treatment group as main effect and corresponding baseline value(s) as covariate, stratified by randomization factors (as per IVRS). Combined estimates of odds ratio versus placebo, 95% CI, and p-value were provided (through SAS MIANALYZE procedure). Safety analyses:

Safety analyses were descriptive, performed on the safety population according to treatment actually received. The safety analysis focused on the TEAE period defined as the time from the first dose of double-blind up to 70 days after the last double-blind injection. TEAE which developed, worsened or became serious or PCSA occurring after the patient inclusion in the open-label extension study (LTS13643) were not considered in the TEAE period. TEAE period was truncated at the common study cut-off date.

### Results

### Study Patients

### Patient Accountability

Of the 107 randomized patients (72 and 35 patients in the alirocumab and the placebo groups, respectively), one patient in the alirocumab group did not have any baseline calculated LDL-C value and was therefore not included in the ITT and mITT populations.

TABLE 26
Analysis populations

| | | Alirocumab | |
|---|---|---|---|
| | Placebo | 150 Q2W | All |
| Randomized population | 35 (100%) | 72 (100%) | 107 (100%) |
| Efficacy populations | | | |
| Intent-to-Treat (ITT) | 35 (100%) | 71 (98.6%) | 106 (99.1%) |
| Modified Intent-to-Treat (mITT) | 35 (100%) | 71 (98.6%) | 106 (99.1%) |
| Safety population | 35 | 72 | 107 |

Note:
The safety population patients are tabulated according to treatment actually received (as treated).
For the other populations, patients are tabulated according to their randomized treatment.

### Study Disposition

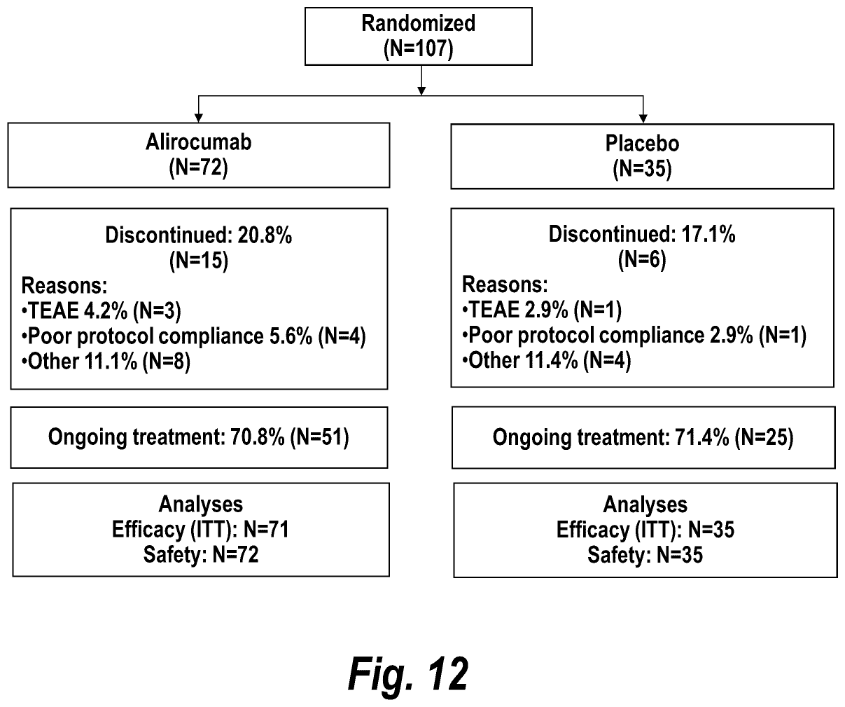

Study disposition, exposure, efficacy and safety analyses were assessed using all data up to the common cut-off date of the study (defined as the date of last patient's Week 52 visit). Therefore, this first step analysis includes efficacy data up to Week 52 and safety data beyond Week 52 and up to Week 78 or Follow-up visit for some patients. Patient disposition is shown in FIG. 12.

In this study, one site with 7 patients randomized and a second site with 6 patients randomized were identified with serious GCP non-compliance, and the sites were closed. For the first closed site, one of the key findings was related to IMP injections reported as having been received by some patients whereas corresponding kits were discovered in the fridge. The reporting of these injections was corrected in the database but other issues on injections could not be excluded. For the second site, persistent concerns with the conduct of the study and associated documentation related to the study were observed during routine monitoring.

Among these 13 patients, one was still ongoing at the cut-off date, one discontinued for adverse event, one patient moved, 3 patients discontinued for poor compliance to protocol and 7 patients discontinued due to decision of site closure.

There were in total 10 (9.3%) randomized patients who completed the 78 weeks double-blind study treatment period and 76 (71.0%) randomized patients with treatment ongoing at the time of the first-step analysis cut-off date. The double-blind IMP was prematurely discontinued before Week 78 for 6 (17.1%) patients in the placebo group and 15 (20.8%) patients in the alirocumab group. All these patients actually prematurely discontinued before Week 52. The main reasons for study treatment discontinuation were “other reasons”, poor compliance and adverse events. These “other reasons” included the 7 patients who discontinued due to the decision of site closure as mentioned above, 1 patient withdrawal not otherwise specified, 1 patient withdrew due to cholesterol results obtained independently and 1 patient moved.

In this first step analysis, final results are available for primary efficacy endpoint at Week 24 and key secondary efficacy endpoints assessed at Week 12, Week 24 and Week 52. The following table provides the availability of LDL-C over time. At Week 24, the primary efficacy endpoint was available for 33 (94.3%) in the placebo and 63 (88.7%) in the alirocumab group.

TABLE 27
Calculated LDL-C availability over time - ITT population

| | Placebo | | | | Alirocumab 150 Q2W | | | |
|---|---|---|---|---|---|---|---|---|
| | (N = 35) | | | | (N = 71) | | | |
| | On- | Post- | | | | Post- | | |
| Calculated | treatment | treatment | Missing | | On-treatment | treatment | Missing | |
| LDL-C | value | value | value | | value | value | value | |
| Week 4 | 31 (88.6%) | 0 | 4 | (11.4%) | 67 (94.4%) | 0 | 4 | (5.6%) |
| Week 8 | 34 (97.1%) | 0 | 1 | (2.9%) | 66 (93.0%) | 0 | 5 | (7.0%) |
| Week 12 | 33 (94.3%) | 0 | 2 | (5.7%) | 68 (95.8%) | 0 | 3 | (4.2%) |
| Week 16 | 28 (80.0%) | 0 | 7 | (20.0%) | 66 (93.0%) | 0 | 5 | (7.0%) |
| Week 24 | 33 (94.3%) | 0 | 2 | (5.7%) | 62 (87.3%) | 1 (1.4%) | 8 | (11.3%) |
| Week 36 | 30 (85.7%) | 1 (2.9%) | 4 | (11.4%) | 60 (84.5%) | 3 (4.2%) | 8 | (11.3%) |
| Week 52 | 27 (77.1%) | 0 | 8 | (22.9%) | 52 (73.2%) | 2 (2.8%) | 17 | (23.9%) |

The primary endpoint was missing for 10 patients at Week 24 (2 and 8 patients in placebo and alirocumab groups, respectively). At the Week 24 visit (as per CRF monitoring), the reasons for missingness were as follows: 3 samples not done due to earlier study discontinuation; 3 samples done outside analysis time window; 2 samples not done due to Week 24 visit not done; and 2 samples available but measurement could not be done (lipemia, insufficient quantity, TGs >400 mg/dL[>4.52 mmol/L], sample lost, etc.).

The higher number of missing data at Week 52 is mainly due to the decision to close the two sites due to serious GCP non-compliance.

The LDL-C endpoint at Week 52 was missing for 25 out of 106 patients. The reasons for missing results were as follows: 17 samples not done due to earlier study discontinuation including 11 patients from the two closed sites; 3 samples done outside analysis time window; 1 sample not done due to Week 52 not done; 1 missing sample while visit Week 52 was done; and 3 samples available but measurement could not be done (TGs >400 mg/dL[>4.52 mmol/L] and hemolysis).

### Demographics and Baseline Characteristics

### Summary Population Characteristics:

107 HeFH patients diagnosed by genotyping (17.8%) and WHO/Dutch Lipid Network criteria (score of >8 points) or Simon Broome criteria for definite FH (82.2%) were randomized 2:1 to alirocumab (150 mg Q2W) or placebo.

Demographics characteristics, disease characteristics and lipid parameters at baseline were generally similar in the alirocumab group as compared to the placebo group: diagnosis of HeFH through genotyping in the alirocumab (19.4%) vs the placebo group (14.3%); diagnosis of HeFH through clinical criteria in the alirocumab (80.6%) vs the placebo group (85.7%); a mean age (SD) in the alirocumab group of 49.8 years (14.2) vs a mean age of the placebo group of 52.1 years (11.2); percentage of white race in the alirocumab (88.9%) vs the placebo (85.7%) group; and a mean BMI (SD) in the alirocumab group of 28.8 kg/m² (5.2) vs a mean BMI in the placebo group of 28.9 kg/m² (4.2). Some imbalances were noted due to the small sample size of the study: a higher proportion of female patients in the alirocumab group (51.4%) vs the placebo group (37.1%); more recent hypercholesterolemia diagnosis in the alirocumab group (median of 9.8 years) vs the placebo group (median of 17.4 years); a lower proportion of patients considered as very high CV risk in the alirocumab group (52.8%) than in the placebo group (65.7%) mainly driven by a medical history of coronary revascularization procedure; and a lower proportion of patients received ezetimibe at randomization in the alirocumab group (19.4%) than in the placebo group (34.3%). The cardiovascular history and risk factors of patients in both the alirocumab and placebo groups are shown in Table 28.

TABLE 28
Cardiovascular history and risk factors

| | Alirocumab | |
|---|---|---|
| Placebo | 150 Q2W | All |
| (N = 35) | (N = 72) | (N = 107) |

All patients on background of maximally tolerated statin ± other LLT

| CHD history, % (n) | 43.1% | (31) | 62.9% | (22) | 49.5% (53) |
|---|---|---|---|---|---|
| Acute MI, % (n) | 22.1% | (16) | 22.9% | (8) | 22.4% (24) |
| Silent MI, % (n) | 1.4% | (1) | 0 | | 0.9% (1) |
| Unstable angina, % (n) | 9.7% | (7) | 17.1% | (6) | 12.1% (13) |
| Coronary revascularization procedures, % (n) | 15.3% | (11) | 40.0% | (14) | 23.4% (25) |
| Other clinically significant CHD, % (n) | 27.8% | (20) | 28.6% | (10) | 28.0% (30) |
| Current smoker, % (n) | 16.7% | (12) | 25.7% | (9) | 19.6% (21) |
| Hypertension, % (n) | 55.6% | (40) | 60.0% | (21) | 57.0% (61) |
| Type 2 diabetes, % (n) | 12.5% | (9) | 17.1% | (6) | 14.0% (15) |

At randomization, all patients were treated with a statin, 72.9% receiving high intensity statin (atorvastatin 40 to 80 mg daily or rosuvastatin 20 to 40 mg daily) and 6.5% receiving simvastatin 80 mg. In addition to the statin, 19.4% and 34.3% of patients were receiving ezetimibe in the alirocumab and placebo groups respectively. Table 30 shows the background lipid modifiying therapies (LMTs) of the alirocumab and placebo treated populations at randomization as well as those of the total randomized population.

Table 31 shows the lipid efficacy parameters at baseline of the alirocumab and placebo treated populations as well as the total randomized population. Mean (SD) calculated LDL-C at baseline was 197.8 (53.4) mg/dL (5.123 (1.38) mmol/L). Mean (SD) non-HDL-C at baseline was 226.4 (55.3) mg/dL. Mean (SD) Total-C at baseline was 274.4 (54.0) mg/dL. Mean (SD) HDL-C at baseline was 48.1 (13.3) mg/dL. The mean (SD) Total-C/HDL-C ratio at baseline was 6.135 (2.119). Mean (SD) fasting triglycerides (TGs) at baseline was 149.8 (86.6) mg/dL. Mean (SD) Lipoprotein-(a) at baseline was 41.2 (46.6) mg/dL. Mean (SD) Apo-B at baseline was 140.9 (31.0) mg/dL. Mean (SD) Apo-A1 at baseline was 137.5 (23.3) mg/dL. The mean (SD) Apo-B/Apo-A1 ratio at baseline was 1.061 (0.323) mg/dL.

Exposure to injections was similar across treatment groups with a mean exposure of 60.7 weeks in placebo group and 58.3 weeks in alirocumab group.

TABLE 29
Disease characteristics and other relevant baseline data - Randomized population

| | | |
|---|---|---|
| | Alirocumab | |
| Placebo | 150 Q2W | All |
| (N = 35) | (N = 72) | (N = 107) |

Type of hypercholesterolemia

| | | | | | | |
|---|---|---|---|---|---|---|
| Heterozygous Familial | 35 | (100%) | 72 | (100%) | 107 | (100%) |
| Hypercholesterolemia (heFH) | | | | | | |

| | | | |
|---|---|---|---|
| Non-Familial | 0 | 0 | 0 |
| Hypercholesterolemia (non-FH) | | | |

Time from hypercholesterolemia diagnosis (years)

| Number | 35 | | 72 | | 107 | |
|---|---|---|---|---|---|---|
| Mean (SD) | 16.41 | (12.62) | 11.48 | (10.48) | 13.09 | (11.41) |

| | | | |
|---|---|---|---|
| Median | 17.42 | 9.76 | 11.70 |
| Min:Max | 0.0:42.8 | 0.0:39.9 | 0.0:42.8 |

Confirmation of diagnosis

| | | | | | | |
|---|---|---|---|---|---|---|
| By genotyping | 5 | (14.3%) | 14 | (19.4%) | 19 | (17.8%) |
| By WHO/Simon Broome^{a} | 30 | (85.7%) | 58 | (80.6%) | 88 | (82.2%) |

^{a}for heFH diagnosis not confirmed by genotyping.

TABLE 30
Background LMT at randomization - Randomized population

| | | | Alirocumab | | | |
|---|---|---|---|---|---|---|
| | Placebo | | 150 Q2W | | All | |
| | (N = 35) | | (N = 72) | | (N = 107) | |
| Any statin | 35 | (100%) | 72 | (100%) | 107 | (100%) |
| Taking high dose statin | 28 | (80.0%) | 57 | (79.2%) | 85 | (79.4%) |
| Taking high intensity statin | 25 | (71.4%) | 53 | (73.6%) | 78 | (72.9%) |
| Atorvastatin daily dose (mg) | 10 | (28.6%) | 22 | (30.6%) | 32 | (29.9%) |
| 10 | 0 | | 0 | | 0 | |
| 20 | 0 | | 0 | | 0 | |
| 40 | 3 | (8.6%) | 10 | (13.9%) | 13 | (12.1%) |
| 80 | 7 | (20.0%) | 11 | (15.3%) | 18 | (16.8%) |
| Other doses | 0 | | 1 | (1.4%) | 1 | (0.9%) |
| Rosuvastatin daily dose (mg) | 16 | (45.7%) | 33 | (45.8%) | 49 | (45.8%) |
| 5 | 1 | (2.9%) | 2 | (2.8%) | 3 | (2.8%) |
| 10 | 0 | | 0 | | 0 | |
| 20 | 3 | (8.6%) | 8 | (11.1%) | 11 | (10.3%) |
| 40 | 12 | (34.3%) | 23 | (31.9%) | 35 | (32.7%) |
| Other doses | 0 | | 0 | | 0 | |
| Simvastatin daily dose (mg) | 10 | (28.6%) | 19 | (26.4%) | 29 | (27.1%) |
| 10 | 1 | (2.9%) | 4 | (5.6%) | 5 | (4.7%) |
| 20 | 1 | (2.9%) | 2 | (2.8%) | 3 | (2.8%) |
| 40 | 5 | (14.3%) | 9 | (12.5%) | 14 | (13.1%) |
| 80 | 3 | (8.6%) | 4 | (5.6%) | 7 | (6.5%) |
| Other doses | 0 | | 0 | | 0 | |
| Any LMT other than statins^{a} | 13 | (37.1%) | 16 | (22.2%) | 29 | (27.1%) |
| Any LMT other than nutraceuticals | 12 | (34.3%) | 16 | (22.2%) | 28 | (26.2%) |
| Ezetimibe | 12 | (34.3%) | 14 | (19.4%) | 26 | (24.3%) |

| | | | | | |
|---|---|---|---|---|---|
| Nutraceuticals | 1 | (2.9%) | 0 | 1 | (0.9%) |

^{a}in combination with statins or not.
High intensity statin corresponds to atorvastatin 40 to 80 mg daily or rosuvastatin 20 to 40 mg daily.
High dose statin corresponds to atorvastatin 40 to 80 mg daily, rosuvastatin 20 to 40 mg daily, or simvastatin 80 mg daily.

TABLE 31
Lipid efficacy parameters at baseline - Quantitative
summary in conventional units - Randomized population

| | | | Alirocumab | | | |
|---|---|---|---|---|---|---|
| | Placebo | | 150 Q2W | | All | |
| | (N = 35) | | (N = 72) | | (N = 107) | |
| Calculated LDL-C (mg/dL) | | | | | | |
| Number | 35 | | 71 | | 106 | |
| Mean (SD) | 201.0 | (43.4) | 196.3 | (57.9) | 197.8 | (53.4) |
| Median | 201.0 | | 180.0 | | 181.0 | |
| Q1:Q3 | 166.0:240.0 | | 165.0:224.0 | | 165.0:224.0 | |
| Min:Max | 137:279 | | 89:402 | | 89:402 | |
| Non-HDL-C (mg/dL) | | | | | | |
| Number | 35 | | 72 | | 107 | |
| Mean (SD) | 231.5 | (47.6) | 223.9 | (58.8) | 226.4 | (55.3) |
| Median | 226.0 | | 204.0 | | 209.0 | |
| Q1:Q3 | 194.0:274.0 | | 189.5:251.0 | | 191.0:260.0 | |
| Min:Max | 153:326 | | 117:419 | | 117:419 | |
| Total-C (mg/dL) | | | | | | |
| Number | 35 | | 72 | | 107 | |
| Mean (SD) | 276.4 | (46.8) | 273.5 | (57.5) | 274.4 | (54.0) |
| Median | 272.0 | | 256.0 | | 259.0 | |
| Q1:Q3 | 237.0:313.0 | | 242.5:300.5 | | 241.0:310.0 | |
| Min:Max | 202:364 | | 171:458 | | 171:458 | |
| HDL-C (mg/dL) | | | | | | |
| Number | 35 | | 72 | | 107 | |
| Mean (SD) | 44.9 | (11.3) | 49.6 | (14.0) | 48.1 | (13.3) |
| Median | 42.0 | | 45.5 | | 45.0 | |
| Q1:Q3 | 39.0:51.0 | | 39.5:57.5 | | 39.0:55.0 | |
| Min:Max | 24:72 | | 28:84 | | 24:84 | |
| Fasting TGs (mg/dL) | | | | | | |
| Number | 35 | | 72 | | 107 | |
| Mean (SD) | 156.3 | (89.3) | 146.6 | (85.6) | 149.8 | (86.6) |
| Median | 122.0 | | 131.5 | | 129.0 | |
| Q1:Q3 | 95.0:193.0 | | 87.5:160.5 | | 94.0:171.0 | |
| Min:Max | 62:455 | | 40:512 | | 40:512 | |
| Lipoprotein- | | | | | | |
| (a)(mg/dL) | | | | | | |
| Number | 34 | | 71 | | 105 | |
| Mean (SD) | 46.2 | (50.3) | 38.8 | (44.9) | 41.2 | (46.6) |
| Median | 30.0 | | 22.0 | | 26.0 | |
| Q1:Q3 | 11.0:42.0 | | 8.0:50.0 | | 10.0:48.0 | |
| Min:Max | 2:201 | | 2:189 | | 2:201 | |
| Apo-B (mg/dL) | | | | | | |
| Number | 34 | | 71 | | 105 | |
| Mean (SD) | 146.6 | (28.3) | 138.2 | (32.0) | 140.9 | (31.0) |
| Median | 143.0 | | 130.0 | | 134.0 | |
| Q1:Q3 | 121.0:173.0 | | 118.0:154.0 | | 119.0:158.0 | |
| Min:Max | 99:208 | | 81:255 | | 81:255 | |
| Apo-A1 (mg/dL) | | | | | | |
| Number | 34 | | 71 | | 105 | |
| Mean (SD) | 131.5 | (19.2) | 140.3 | (24.6) | 137.5 | (23.3) |
| Median | 127.5 | | 137.0 | | 134.0 | |
| Q1:Q3 | 120.0:142.0 | | 122.0:155.0 | | 122.0:151.0 | |
| Min:Max | 97:181 | | 97:211 | | 97:211 | |
| Apo-B/Apo-A1 (ratio) | | | | | | |
| Number | 34 | | 71 | | 105 | |
| Mean (SD) | 1.141 | (0.287) | 1.023 | (0.334) | 1.061 | (0.323) |
| Median | 1.170 | | 0.950 | | 1.020 | |
| Q1:Q3 | 0.900:1.300 | | 0.800:1.170 | | 0.850:1.230 | |
| Min:Max | 0.58:1.86 | | 0.49:2.32 | | 0.49:2.32 | |
| Total-C/HDL-C (ratio) | | | | | | |
| Number | 35 | | 72 | | 107 | |
| Mean (SD) | 6.540 | (1.986) | 5.938 | (2.167) | 6.135 | (2.119) |

| | | | |
|---|---|---|---|
| Median | 6.417 | 5.647 | 5.863 |
| Q1:Q3 | 4.936:7.600 | 4.399:6.878 | 4.545:7.370 |
| Min:Max | 3.29:11.19 | 2.92:13.48 | 2.92:13.48 |

### Dosage and Duration

Exposure to injections was similar across treatment groups with a mean exposure of 60.7 weeks in the placebo group and 58.3 weeks in the alirocumab group. Duration of exposure for injection could not be calculated for 1 patient in alirocumab group due to unknown last injection date.

### Efficacy

### Primary Efficacy Endpoint

The ITT analysis includes all calculated LDL-C values collected on-treatment and off-treatment up to Week 52. The primary endpoint (percent change in calculated LDL-C from baseline to Week 24) analysis is provided based on a MMRM model on the ITT population, using LS means estimates at Week 24. Nine (11.3%) patients in the alirocumab group and 2 (5.7%) patients in the placebo group did not have a calculated LDL-C value at Week 24. These missing values were accounted for by the MMRM model.

Results of the primary endpoint analysis are presented in Table 32, in mmol/L and mg/dL.

### Primary Efficacy Analysis

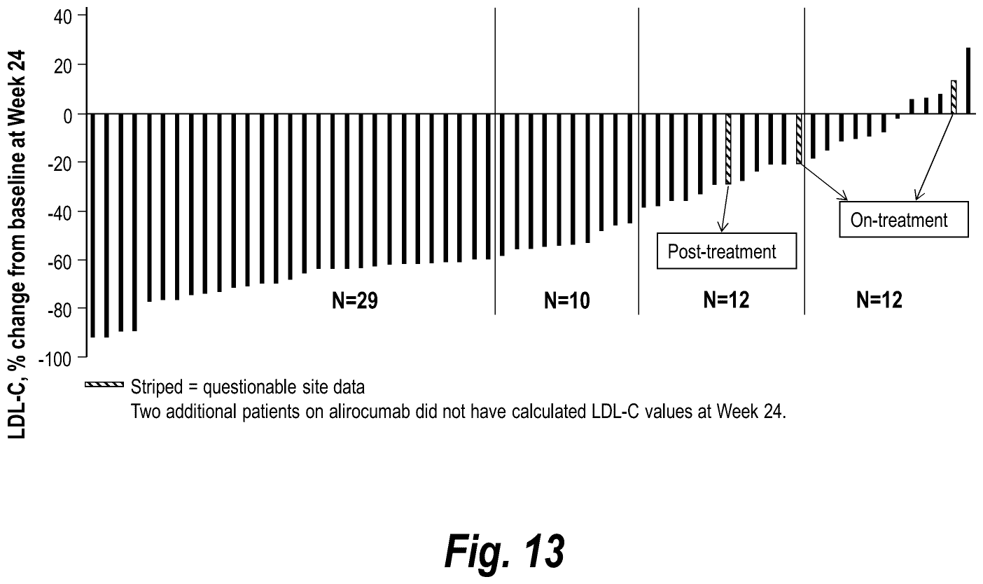

A statistically significant decrease in percent change in LDL-C from baseline to Week 24 was observed in the alirocumab group (LS mean versus baseline −45.7%) compared to the placebo group (LS mean versus baseline −6.6%) (LS mean difference vs. placebo (SE) of −39.1% (6.0%), p<0.0001) (see Table 31). This represents an absolute reduction (SD) of −90.8 (6.7) mg/dL in the alirocumab group and −15.5 (9.5) mg/dL in the placebo group (see Table 33). Percent change from baseline to Week 24 in LDL-C by individual patients are set forth in FIG. 13. All patients were on a background statin (at the maximum tolerated level). A subset of patients also received a further lipid lowering therapy.

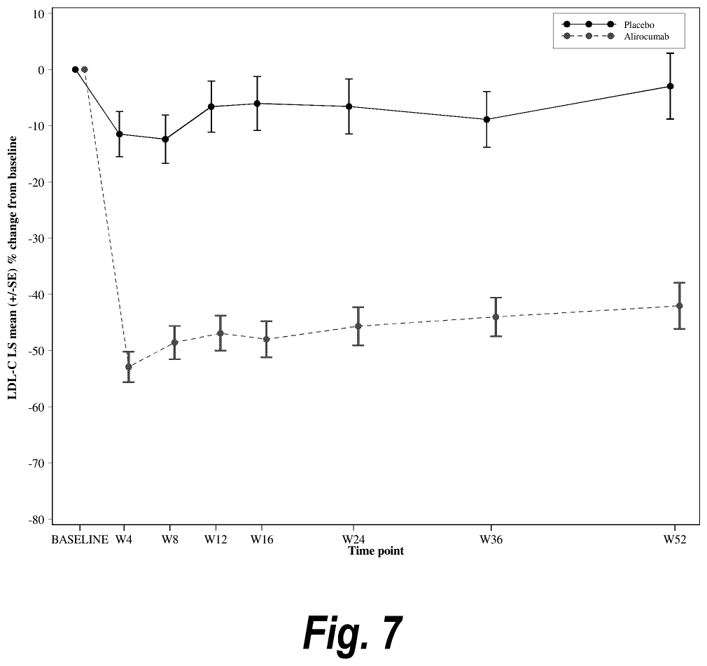

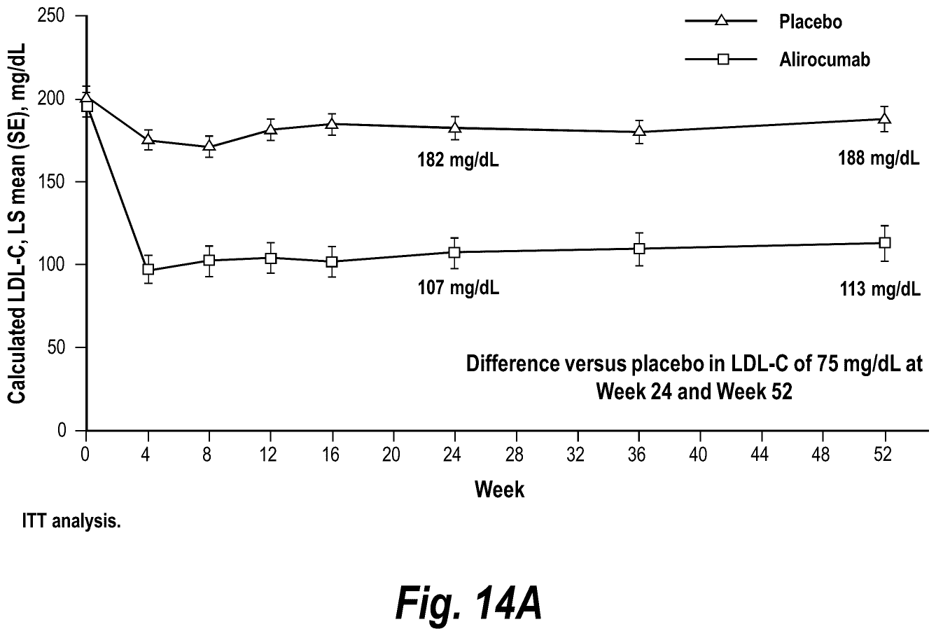

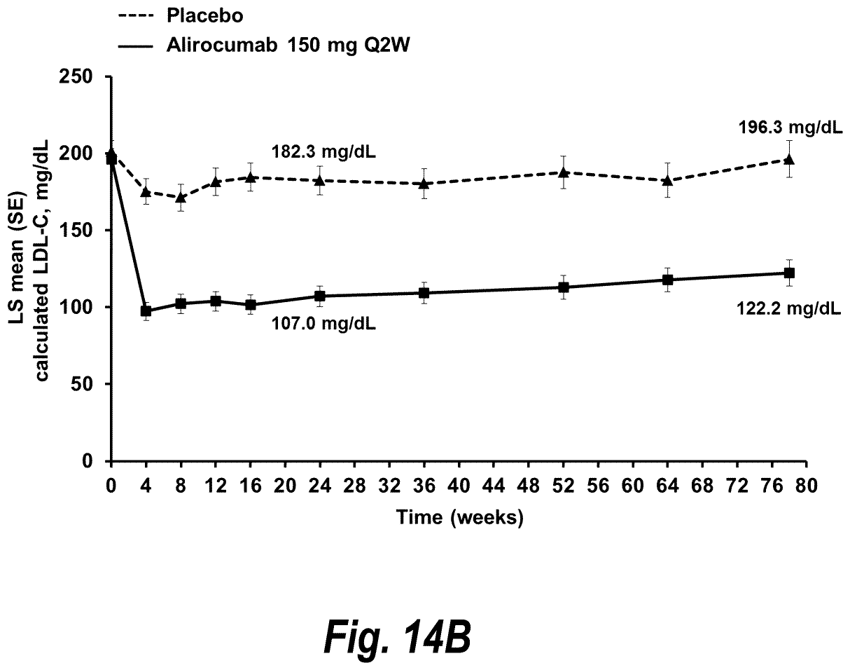

In the alirocumab group, LDL-C reduction from baseline was observed from Week 4 to Week 52 (see FIG. 7, FIGS. 14A-14B and Table 33). A slight decrease in LDL-C reduction over time was observed in the alirocumab group (LS mean versus baseline at Week 52 of −42.1 versus −45.7 at Week 24), although the overall amount of the decrease stayed the same (75 mg/dL; see FIGS. 14A-14B). Furthermore, significant numbers of patients on alirocumab achieved LDL-C levels of <100 mg/dL (57% vs 11% of placebo patients) and <70 mg/dL (<1.81 mmol/L; 32% vs 3% of placebo patients) at Week 24 despite baseline LDL-C levels of >190 mg/dL (mean (SD) baseline calculated LDL-C 196.3 (57.9) mg/dL for alirocumab group; 201 (43.4) mg/dL for placebo group). At week 12, 31.0% of alirocumab group patients (vs. 0.0% of placebo group; ITT analysis) reached calculated LDL-C levels of <70 mg/dL (<1.81 mmol/L). Similarly, at Week 52, 31.0% of alirocumab group patients (vs 5.7% of placebo group; ITT analysis) reached calculated LDL-C levels of <70 mg/dL (<1.81 mmol/L).

A sensitivity analysis of the primary efficacy endpoint was performed excluding 13 patients from 2 sites with serious GCP non compliance. The decrease in percent change in LDL-C from baseline to Week 24 was still statistically significant in the alirocumab group (LS mean versus baseline −50.3%) compared to the placebo group (LS mean versus baseline −2.3%) (LS mean difference vs. placebo (SE) of −48.0% (5.8%), p<0.0001) (see Table 34).

TABLE 32
Percent change from baseline in calculated LDL-C
at Week 24: MMRM - ITT analysis - ITT population

| | | |
|---|---|---|
| | | Alirocumab |
| | Placebo | 150 Q2W |
| Calculated LDL Cholesterol | (N = 35) | (N = 71) |

Baseline (mmol/L)

| Number | 35 | | 71 | |
|---|---|---|---|---|
| Mean (SD) | 5.205 | (1.125) | 5.083 | (1.499) |

| | | |
|---|---|---|
| Median | 5.206 | 4.662 |
| Min:Max | 3.55:7.23 | 2.31:10.41 |

Baseline (mg/dL)

| Number | 35 | | 71 | |
|---|---|---|---|---|
| Mean (SD) | 201.0 | (43.4) | 196.3 | (57.9) |

| | | |
|---|---|---|
| Median | 201.0 | 180.0 |
| Min:Max | 137:279 | 89:402 |

Week 24 percent change from baseline (%)

| | | | | |
|---|---|---|---|---|
| LS Mean (SE) | −6.6 | (4.9) | −45.7 | (3.5) |
| LS mean difference (SE) vs placebo | | | −39.1 | (6.0) |

| | |
|---|---|
| 95% CI | (−51.1 to −27.1) |
| p-value vs placebo | <0.0001* |

Note:
Least-squares (LS) means, standard errors (SE) and p-value taken from MMRM (mixed-effect model with repeated measures) analysis. The model includes the fixed categorical effects of treatment group, randomization strata as per IVRS, time point, treatment-by-time point and strata-by-time point interaction, as well as the continuous fixed covariates of baseline calculated LDL-C value and baseline calculated LDL-C value-by-time point interaction
MMRM model and baseline description run on patients with a baseline value and a post-baseline value in at least one of the analysis windows used in the model.
The p-value is followed by a ‘*’ if statistically significant according to the fixed hierarchical approach used to ensure a strong control of the overall type-I error rate at the 0.05 level

TABLE 33
Calculated LDL-C overtime - ITT analysis - ITT population

| | Placebo | | | | | | Alirocumab 150 Q2W | | | | |
|---|---|---|---|---|---|---|---|---|---|---|---|
| | (N = 35) | | | | | | (N = 71) | | | | |
| | | | | | Percent | | | | | | Percent |
| | | | Change | | change | | | | Change | | change |
| Calculated | | | from | | from | | | | from | | from |
| LDL-C | Value | | baseline | | baseline | | Value | | baseline | | baseline |
| LS Mean (SE) (mmol/L) | | | | | | | | | | | |
| Baseline ^{a} | 5.205 | (0.190) | NA | | NA | | 5.083 | (0.178) | NA | | NA |
| Week 4 | 4.537 | (0.221) | −0.586 | (0.221) | −11.5 | (4.1) | 2.522 | (0.154) | −2.601 | (0.154) | −52.9 (2.8) |
| Week 8 | 4.435 | (0.229) | −0.688 | (0.229) | −12.4 | (4.3) | 2.647 | (0.161) | −2.477 | (0.161) | −48.6 (3.1) |
| Week 12 | 4.702 | (0.234) | −0.422 | (0.234) | −6.6 | (4.6) | 2.692 | (0.164) | −2.432 | (0.164) | −46.9 (3.2) |
| Week 16 | 4.779 | (0.235) | −0.344 | (0.235) | −6.1 | (4.8) | 2.633 | (0.161) | −2.490 | (0.161) | −48.0 (3.3) |
| Week 24 | 4.722 | (0.246) | −0.401 | (0.246) | −6.6 | (4.9) | 2.771 | (0.174) | −2.352 | (0.174) | −45.7 (3.5) |
| Week 36 | 4.666 | (0.251) | −0.457 | (0.251) | −8.9 | (5.0) | 2.832 | (0.176) | −2.292 | (0.176) | −44.0 (3.5) |
| Week 52 | 4.862 | (0.275) | −0.262 | (0.275) | −3.0 | (5.9) | 2.921 | (0.197) | −2.202 | (0.197) | −42.1 (4.2) |
| Week 78 | | | | | 1.2 | (6.4) | | | | | −37.9 (4.5) |
| LS Mean (SE) (mg/dL) | | | | | | | | | | | |
| Baseline ^{a} | 201.0 | (7.3) | NA | | NA | | 196.3 | (6.9) | NA | | NA |
| Week 4 | 175.2 | (8.5) | −22.6 | (8.5) | −11.5 | (4.1) | 97.4 | (5.9) | −100.4 | (5.9) | −52.9 (2.8) |
| Week 8 | 171.2 | (8.8) | −26.6 | (8.8) | −12.4 | (4.3) | 102.2 | (6.2) | −95.6 | (6.2) | −48.6 (3.1) |
| Week 12 | 181.5 | (9.0) | −16.3 | (9.0) | −6.6 | (4.6) | 103.9 | (6.3) | −93.9 | (6.3) | −46.9 (3.2) |
| Week 16 | 184.5 | (9.1) | −13.3 | (9.1) | −6.1 | (4.8) | 101.7 | (6.2) | −96.1 | (6.2) | −48.0 (3.3) |
| Week 24 | 182.3 | (9.5) | −15.5 | (9.5) | −6.6 | (4.9) | 107.0 | (6.7) | −90.8 | (6.7) | −45.7 (3.5) |
| Week 36 | 180.2 | (9.7) | −17.7 | (9.7) | −8.9 | (5.0) | 109.3 | (6.8) | −88.5 | (6.8) | −44.0 (3.5) |
| Week 52 | 187.7 | (10.6) | −10.1 | (10.6) | −3.0 | (5.9) | 112.8 | (7.6) | −85.0 | (7.6) | −42.1 (4.2) |

^{a} Baseline is described using means and standard errors.
Note:
Least-squares (LS) means, standard errors (SE) and p-value taken from MMRM (mixed-effect model with repeated measures) analysis. The model includes the fixed categorical effects of treatment group, randomization strata as per IVRS, time point, treatment-by-time point interaction, strata-by-time point interaction, as well as the continuous fixed covariates of baseline LDL-C value and baseline LDL-C value-by-time point interaction
MMRM model and baseline description run on patients with a baseline value and a post-baseline value in at least one of the analysis windows used in the model.

### Sensitivity Analysis of Primary Endpoint

TABLE 34
Percent change from baseline in calculated LDL-C
at Week 24: MMRM - ITT analysis - ITT population
excluding sites with serious GCP non compliance

| | | | Alirocumab | |
|---|---|---|---|---|
| Calculated LDL | Placebo | | 150 Q2W | |
| Cholesterol | (N = 31) | | (N = 62) | |
| Baseline (mmol/L) | | | | |
| Number | 31 | | 62 | |
| Mean (SD) | 5.310 | (1.146) | 5.101 | (1.460) |
| Median | 5.258 | | 4.675 | |
| Min:Max | 3.55:7.23 | | 2.31:10.41 | |
| Baseline (mg/dL) | | | | |
| Number | 31 | | 62 | |
| Mean (SD) | 205.0 | (44.2) | 197.0 | (56.4) |
| Median | 203.0 | | 180.5 | |
| Min:Max | 137:279 | | 89:402 | |
| Week 24 percent change from | | | | |
| baseline (%) | | | | |
| LS Mean (SE) | −2.3 | (4.7) | −50.3 | (3.3) |
| LS mean difference (SE) vs placebo | | | −48.0 | (5.8) |

| | |
|---|---|
| 95% CI | (−59.4 to −36.6) |
| p-value vs placebo | <0.0001 |

Note:
Least-squares (LS) means, standard errors (SE) and p-value taken from MMRM (mixed-effect model with repeated measures) analysis. The model includes the fixed categorical effects of treatment group, randomization strata as per IVRS, time point, treatment-by-time point and strata-by-time point interaction, as well as the continuous fixed covariates of baseline calculated LDL-C value and baseline calculated LDL-C value-by-time point interaction
MMRM model and baseline description run on patients with a baseline value and a post-baseline value in at least one of the analysis windows used in the model.
The p-value is not adjusted for multiplicity and provided for descriptive purpose only
Note:
Sites No. 643-710 and No. 840-743 were excluded from analysis

### Key Secondary Efficacy Endpoints

The following table summarizes analysis results on key secondary endpoints in the hierarchical order. All key secondary endpoints are statistically significant according to the hierarchical testing procedure up to Lp(a) endpoint at Week 24 (ITT estimand) included.

Statistically significance was not reached for HDL-C at Week 24 (ITT estimand) and therefore the testing procedure was stopped, p-values are provided from this endpoint for descriptive purpose only.

TABLE 35

| Endpoint | Analysis | Results | P-value |
|---|---|---|---|
| Calculated LDL-C - Percent | On-treatment | LS mean difference vs. | <0.0001 |
| change from baseline to Week | | placebo of −38.9% | |
| 24 | | | |
| Calculated LDL-C - Percent | ITT | LS mean difference vs. | <0.0001 |
| change from baseline to Week | | placebo of −40.3% | |
| 12 | | | |
| Calculated LDL-C - Percent | On-treatment | LS mean difference vs. | <0.0001 |
| change from baseline to Week | | placebo of −40.3% | |
| 12 | | | |
| Apo-B - Percent change from | ITT | LS mean difference vs. | <0.0001 |
| baseline to Week 24 | | placebo of −30.3% | |
| Apo-B - Percent change from | On-treatment | LS mean difference vs. | <0.0001 |
| baseline to Week 24 | | placebo of −30.2% | |
| Non-HDL-C - Percent change | ITT | LS mean difference vs. | <0.0001 |
| from baseline to Week 24 | | placebo of −35.8% | |
| Non-HDL-C - Percent change | On-treatment | LS mean difference vs. | <0.0001 |
| from baseline to Week 24 | | placebo of −35.5% | |
| Total-C - Percent change from | ITT | LS mean difference vs. | <0.0001 |
| baseline to Week 24 | | placebo of −28.4% | |
| Apo-B - Percent change from | ITT | LS mean difference vs. | <0.0001 |
| baseline to Week 12 | | placebo of −30.2% | |
| Non-HDL-C - Percent change | ITT | LS mean difference vs. | <0.0001 |
| from baseline to Week 12 | | placebo of −34.5% | |
| Total-C - Percent change from | ITT | LS mean difference vs. | <0.0001 |
| baseline to Week 12 | | placebo of −27.8% | |
| Calculated LDL-C - Percent | ITT | LS mean difference vs. | <0.0001 |
| change from baseline to Week | | placebo of −39.1% | |
| 52 | | | |
| Proportion of very high CV | ITT | combined estimate for odds- | 0.0016 |
| risk patients reaching | | ratio vs. placebo of 11.7 | |
| calculated LDL-C <70 | | | |
| mg/dL (1.81 mmol/L) or high | | | |
| CV risk patients reaching | | | |
| calculated LDL-C <100 | | | |
| mg/dL (2.59 mmol/L) at | | | |
| Week 24 | | | |
| Proportion of very high CV | On-treatment | combined estimate for odds- | 0.0014 |
| risk patients reaching | | ratio vs. placebo of 11.9 | |
| calculated LDL-C <70 | | | |
| mg/dL (1.81 mmol/L) or high | | | |
| CV risk patients reaching | | | |
| calculated LDL-C <100 | | | |
| mg/dL (2.59 mmol/L) at | | | |
| Week 24 | | | |
| Lp(a) - Percent change from | ITT | combined estimate for adjusted | 0.0164 |
| baseline to Week 24 | | mean difference vs. placebo | |
| | | of −14.8% | |
| HDL-C - Percent change from | ITT | LS mean difference vs. placebo | 0.2745 |
| baseline to Week 24 | | of 3.7% | |
| Fasting TGs - Percent change | ITT | combined estimate for adjusted | 0.1386 |
| from baseline to Week 24 | | mean difference vs. placebo | |
| | | of −8.7% | |

The on-treatment analysis of the LDL-C percent change from baseline to Week 24 shows very consistent results with the ITT analysis (LS mean difference vs. placebo of −38.9% in the on-treatment analysis versus −39.1% in the ITT analysis). Indeed, only 3 patients (2 in placebo and 1 in alirocumab) had LDL-C values collected post-treatment (ie more than 21 days after last injection) at Week 24.

The key secondary endpoints including Apo B, non-HDL-C, Total-C, Lp(a) at various time points as well as the proportion of patients reaching their LDL-C goals at Week 24 were statistically significant according to the hierarchical testing procedure. Significant reductions were seen in non-HDL-C, Apo B, and Lp(a) levels at Week 24. The alirocumab vs placebo LS mean percent change from baseline to week 24 was −41.9 vs −6.2 for non-HDL-C (p value <0.0001), −39.0 vs −8.7 for Apo B (p value <0.0001), and −23.5 vs −8.7 for Lp(a) (p value=0.0164).

The proportion of very high cardiovascular (CV) risk patients reaching calculated LDL-C<70 mg/dL (1.81 mmol/L) or high CV risk patients reaching calculated LDL-C<100 mg/dL (2.59 mmol/L) at Week 24 was significantly higher in the alirocumab than in the placebo group (combined estimate for proportion of 41.0% in the alirocumab group vs 5.7% in the placebo group, p=0.0016).

Analyses performed with on-treatment approach were constitent with these analyses.

The differences in percent change in HDL-C and fasting TGs from baseline to Week 24 in the ITT analysis were non-statistically significant: HDL-C at Week 24: LS mean versus baseline was +7.5% in the alirocumab group and +3.9% in the placebo group (LS mean difference vs. placebo of +3.7%, p=0.2745); and Fasting TGs at Week 24: LS mean versus baseline was −10.5% in the alirocumab group and −1.1% in the placebo group (LS mean difference vs. placebo of −9.4%, p=0.1299).

Four (5.6%) patients experienced two consecutive calculated LDL-C values <25 mg/dL. No particular safety concern has been observed in these patients.

### Summary Safety Results:

The proportion of patients who experienced a treatment emergent adverse event (TEAE) was lower in the alirocumab group (61.1%) compared to placebo group (71.4%) in the present study. The proportion of patients who experienced a serious TEAE was similar between treatment groups. A similar proportion of patients experienced TEAEs leading to treatment discontinuation (1 patient (2.9%) and 3 patients (4.2%) in the placebo and alirocumab groups, respectively). These results are consistent with the proportion of patients who have experienced TEAEs in previous alirocumab Phase 2/3 placebo-controlled studies (results from 2476 and 1276 patients in the alirocumab and placebo groups, respectively). Specifically, in this study TEAEs were 75.8% vs 76.4%, treatment-emergent SAEs were 13.7% vs 14.3%, TEAEs leading to death were 0.5% vs 0.9%, and TEAEs leading to discontinuation were 5.3% vs 5.1%, for alirocumab vs. placebo groups, respectively.

The most frequently reported SOC (and PT) in both treatment groups of the present study were: “infections and infestations”: 40.3% in the alirocumab group vs 34.3% in the placebo group (with influenza reported in 11.1% vs 2.9% and urinary tract infection in 6.9% vs 0 in alirocumab vs placebo group respectively); “cardiac disorders”: 12.5% in the alirocumab group vs no case in the placebo group. Among the events sent to adjudication, events were confirmed for 6 patients presenting: 4 MI, 1 heart failure requiring hospitalization and 5 ischemia driven coronary revascularization procedures; “nervous system disorders”: 11.1% in the alirocumab group vs 8.6% in the placebo group (with headache reported in 5.6% vs 0 and dizziness 4.2% vs 0 in alirocumab vs placebo group respectively); and “musculoskeletal and connective tissue disorders”: 16.7% in the alirocumab group vs 28.6% in the placebo group. No death was reported during the study in either group.

SAEs were reported by 11.1% patients in the alirocumab group and 11.4% in the placebo group. There is no particular clinical pattern among SAEs preferred terms which were individually reported. The most frequently reported SOC (system organ class) for SAEs is “cardiac disorders”.

Seven patients, 6 (8.3%) in the alirocumab group and 1 (2.9%) in the placebo group experienced a treatment-emergent local injection site reaction. These events were of mild intensity except one of moderate intensity. Two patients, one (1.4%) in the alirocumab group and one (2.9%) in the placebo group experienced neurocognitive disorders. Four patients, three (4.2%) in the alirocumab group and one (2.9%) in the placebo group experienced ALT >3×ULN. Two patients out of 71 analysed (2.8%, in comparison to 0 patients in the placebo group) experienced a creatine kinase level >3×ULN. None of the events were serious or led to treatment discontinuation. TEAEs occurring in alirocumab and placebo patient groups were collected until the last patient visit at Week 52 and are categorized in Table 36.

TABLE 36
TEAE safety analysis through week 52.

| | |
|---|---|
| | Alirocumab |
| Placebo | 150 Q2W |
| (N = 35) | (N = 72) |

% (n) of patients
All patients on background of maximally
tolerated statin ± other LLT

| | | |
|---|---|---|
| Nasopharyngitis | 11.4% (4) | 11.1% (8) |
| Influenza | 2.9% (1) | 11.1% (8) |
| Injection-site reaction | 2.9% (1) | 8.3% (6) |
| Urinary tract infection | 0 | 6.9% (5) |
| Diarrhea | 8.6% (3) | 5.6% (4) |
| Sinusitis | 5.7% (2) | 5.6% (4) |
| Bronchitis | 2.9% (1) | 5.6% (4) |
| Headache | 0 | 5.6% (4) |
| Fatigue | 0 | 5.6% (4) |
| Myalgia | 8.6% (3) | 4.2% (3) |
| Nausea | 5.7% (2) | 1.4% (1) |
| Vertigo | 5.7% (2) | 1.4% (1) |
| Dyspepsia | 5.7% (2) | 0 |
| Increased Blood Uric Acid | 5.7% (2) | 0 |
| Rheumatoid arthritis | 5.7% (2) | 0 |

Among the events of interest no particular signal was detected for TEAE related to neurological events, general allergic events and diabetes.

No relevant abnormality for PCSA was observed.

The present invention is not limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### Conclusion

The following conclusions regarding patients with HeFH and high baseline levels of LDL-C despite maximally tolerated statin with or without another LLT can be drawn from the ODYSSEY HIGH FH study: 1) self-administered alirocumab produced significantly greater LDL-C reductions vs. placebo after 24 weeks, with absolute mean decreasing from baseline in LDL-C was −90.8 mg/dL at Week 24 with alirocumab versus −15.5 mg/dL with placebo, and resultant LDL-C levels of 107 mg/dL with alirocumab at Week 24 versus 182 mg/dL with placebo; 2) 32% of alirocumab patients reached LDL-C<70 mg/dL despite baseline LDL-C>190 mg/dL; 3) 57% of alirocumab patients achieved LDL-C<100 mg/dL at Week 24; 4) alirocumab was generally well tolerated and TEAEs occurred in a similar frequency in the alirocumab and placebo arms.

### Example 5: Efficacy and Safety of the PCSK9 Monoclonal Antibody Alirocumab Vs Placebo in 1254 Patients with Heterozygous Familial Hypercholesterolemia (HeFH): Analyses Up to 78 Weeks from Four ODYSSEY Trials

### Background

Previous studies have shown that only ˜20% of heterozygous familial hypercholesterolemia (HeFH) patients treated with lipid-lowering therapies (LLTs) achieved pre-defined LDL-C target levels of ≤2.5 mmol/L [97 mg/dL]. The efficacy and safety of alirocumab vs placebo was studied in 1254 HeFH pts on maximally-tolerated statin±other LLT from four, 18-month, placebo-controlled ODYSSEY trials (FHI, FHII, HIGH FH, LONG TERM). This represents the single largest collection of patients with HeFH studied in a Phase 3 clinical trials program. A description of the LONG TERM study is set forth in Robinson et al., (2015) NEJM 372:16 pg 1489-99, which is incorporated by reference herein in its entirety.

### Methods

Data were pooled by initial alirocumab dose. In FH I/II, patients with LDL-C levels ≥1.81/2.59 mmol/L [70/100 mg/dL], depending on CV risk, received placebo (N=244) or alirocumab 75 mg Q2W (N=488); the alirocumab dose was increased to 150 mg Q2W at week 12 if LDL-C at week 8≥1.81 mmol/L [70 mg/dL](41.8% of patients). Separately, data was pooled from HIGH FH (LDL-C≥4.14 mmol/L [160 mg/dL]) and the subset of patients with HeFH from LONG TERM (LDL-C≥1.81 mmol/L [70 mg/dL]), where patients received placebo (N=180) or alirocumab 150 mg Q2W (N=342). All doses were 1-mL subcutaneous (SC) injections. Data for change in LDL-C from baseline was pooled through week 52.

### Results

Baseline LDL-C levels and changes from baseline with treatment are shown in Table 37. Compared to placebo, alirocumab reduced LDL-C by 49% and 61% (p<0.0001) at week 12 for the 75 and 150 mg Q2W doses, respectively. At week 24, LDL-C reductions with alirocumab vs placebo were 56% (alirocumab 75 mg Q2W with a possible week 12 dose increase) and 59% (alirocumab 150 mg Q2W), respectively (p<0.0001). For both dose regimens, despite high baseline LDL-C levels, LS mean LDL-C levels of ˜2 mmol/L [77 mg/dL] were achieved by week 12 (Table 37), with reductions maintained through Week 52. Additional beneficial effects were observed in other parameters including non-HDL-C and Apo B.

In the individual studies to date, generally similar rates of treatment-emergent adverse events (TEAEs) were observed in alirocumab and placebo-treated patients. Across placebo-controlled studies in the ODYSSEY Program (patients both with and without HeFH), TEAEs (preferred terms) reported in ≥5% of alirocumab or placebo patients include nasopharyngitis (11.3% and 11.1% of alirocumab and placebo-treated patients, respectively), upper respiratory tract infection (URI) (6.1% vs 7.0%), injection site reaction (6.7% vs 4.8%), influenza (5.7% vs 4.6%), headache (4.8% vs 5.2%) and arthralgia (4.0% vs 5.5%).

TABLE 37
Least-squares (LS) mean (SE) calculated LDL-C at week 12 (W 12),
week 24 (W 24) and week 52 (W 52) (intent-to-treat analyses)

| | | | | Alirocumab 75/150 mg | | | |
|---|---|---|---|---|---|---|---|
| | Placebo (N = 244) | | | Q2W (N = 488) | | | |
| | | Change | % | | Change | % | % |
| Pool of FHI | Calculated | from | change | Calculated | from | change | difference |
| and FHII | LDL-C, | baseline, | from | LDL-C, | baseline, | from | versus |
| studies† | mmol/L | mmol/L | baseline | mmol/L | mmol/L | baseline | placebo |
| Baseline, | 3.65 (0.08) | — | — | 3.66 (0.06) | — | — | — |
| mean (SE) | | | | | | | |
| W 12 | 3.80 (0.06) | 0.14 (0.06) | 5.4 (1.6) | 2.04 (0.04) | −1.62 (0.04) | −43.6 (1.1)* | −49.0 (1.9)* |
| W 24 | 3.86 (0.07) | 0.21 (0.07) | 7.1 (1.7) | 1.82 (0.05) | −1.84 (0.05) | −48.8 (1.2)* | −55.8 (2.1)* |
| W 52 | 3.90 (0.07) | 0.25 (0 07) | 8.8 (2.0) | 1.85 (0.05) | −1.81 (0.05) | −48.2 (1.5)* | −57.0 (2.5)* |
| Pool of | | | | | | | |
| LONG TERM | | | | | | | |
| (HeFH | | | | | | | |
| patients | | | | | | | |
| only) and | | | | Alirocumab 150 mg | | | |
| HIGH FH‡ | Placebo (N = 180) | | | Q2W (N = 342) | | | |
| Baseline, | 3.99 (0.11) | — | — | 4.16 (0.09) | — | — | — |
| mean (SE) | | | | | | | |
| W 12 | 4.03 (0.08) | −0.07 (0.08) | 1.9 (1.7) | 1.75 (0.06) | −2.35 (0.06) | −58.8 (1.3)* | −60.7 (2.1)* |
| W 24 | 4.03 (0.08) | −0.07 (0.08) | 2.6 (1.9) | 1.86 (0.06) | −2.24 (0.06) | −56.3 (1.4)* | −58.9 (2.4)* |
| W 52 | 4.19 (0.10) | 0.09 (0.10) | 6.2 (2.5) | 1.94 (0.07) | −2.16 (0.07) | −53.4 (1.8)* | −59.6 (3.1)* |

†alirocumab dose 75 mg Q2W, increasing to 150 mg Q2W at W 12 if LDL-C at W 8 ≥1.81 mmol/L;
‡alirocumab dose 150 mg Q2W;
*p < 0.0001 vs placebo

### Conclusions

In this large cohort of 1254 pts with HeFH, alirocumab reduced mean LDL-C levels to <2 mmol/L [77 mg/dL] at week 24-52 of treatment, levels hitherto unobtainable with current LLTs.

1-92. (canceled)

93. A method for treating hypercholesterolemia in a patient in need thereof having heterozygous familial hypercholesterolemia and having a serum LDL-C concentration of greater than or equal to 70 mg/dL comprising initially administering one or more times, to the patient, 75 mg of an antibody or antigen-binding fragment thereof which specifically binds human PCSK9 and comprises HCDR1, HCDR2 and HCDR3 of a heavy chain variable region comprising the amino acid sequence set forth in SEQ ID NO: 1 and LCDR1, LCDR2 and LCDR3 of a light chain variable region comprising the amino acid sequence set forth in SEQ ID NO: 6, every two weeks; and, then, starting two weeks after the last dose of 75 mg, administering one or more times, to the patient, 150 mg of said antibody or antigen-binding fragment every two weeks.

94. The method of claim 93, wherein the antibody or antigen-binding fragment thereof wherein the
HCDR1 comprises the amino acid sequence set forth in SEQ ID NO: 2;
HCDR2 comprises the amino acid sequence set forth in SEQ ID NO: 3;
HCDR3 comprises the amino acid sequence set forth in SEQ ID NO: 4;
LCDR1 comprises the amino acid sequence set forth in SEQ ID NO: 7;
LCDR2 comprises the amino acid sequence set forth in SEQ ID NO: 8; and
LCDR3 comprises the amino acid sequence set forth in SEQ ID NO: 10.

95. The method of claim 93, wherein the an antibody or antigen-binding fragment thereof comprises a heavy chain variable region comprising the amino acid sequence set forth in SEQ ID NO: 1 and a light chain variable region comprising the amino acid sequence set forth in SEQ ID NO: 6.

96. The method of claim 93, wherein the antibody or antigen-binding fragment thereof is an antibody.

97. The method of claim 93, wherein the antibody or antigen-binding fragment thereof is alirocumab.

98. The method of claim 97, wherein the patient has a serum LDL-C of greater than or equal to 100 mg/dl.

99. The method of claim 97, wherein the patient has a serum LDL-C of greater than or equal to 160 mg/dl.

100. The method of claim 97, wherein the patient has heterozygous familial hypercholesterolemia that is not adequately controlled by a maximally tolerated statin therapy.

101. The method of claim 100, wherein the maximally tolerated statin therapy comprises a daily dose of about 40 mg to about 80 mg of atorvastatin, a daily dose of about 20 mg to about 40 mg of rosuvastatin, or a daily dose of about 80 mg of simvastatin.

102. The method of claim 100, wherein the antibody or fragment is administered to the patient in combination with the maximally tolerated statin therapy.

103. The method of claim 102, wherein the maximally tolerated statin therapy comprises a daily dose of about 40 mg to about 80 mg of atorvastatin, a daily dose of about 20 mg to about 40 mg of rosuvastatin, or a daily dose of about 80 mg of simvastatin.

104. The method of claim 102, wherein the patient has one or more of the indications selected from the group consisting of diabetes mellitus, hypertension, myocardial infarction, and ischemic stroke.

105. The method of claim 93, wherein, prior to receiving a first dose of 150 mg, the patient has an LDL-C level of greater than or equal to 70 mg/dl.

106. The method of claim 93, for treating hypercholesterolemia in a patient in need thereof having heterozygous familial hypercholesterolemia and having a serum LDL-C concentration of greater than or equal to 70 mg/dL comprising initially administering, to the patient, 75 mg of an antibody or antigen-binding fragment thereof which specifically binds human PCSK9 and comprises HCDR1, HCDR2 and HCDR3 of a heavy chain variable region comprising the amino acid sequence set forth in SEQ ID NO: 1 and LCDR1, LCDR2 and LCDR3 of a light chain variable region comprising the amino acid sequence set forth in SEQ ID NO: 6, every two weeks;
and, then, after 6 doses, starting two weeks after the last dose of 75 mg, administering, one or more times, to the patient, 150 mg of said antibody or antigen-binding fragment every two weeks.

107. The method of claim 93, for treating hypercholesterolemia in a patient in need thereof having heterozygous familial hypercholesterolemia that is not adequately controlled by a maximally tolerated statin therapy and having a serum LDL-C concentration of greater than or equal to 70 mg/dL comprising initially administering one or more times, to the patient, 75 mg of alirocumab, by subcutaneous injection, every two weeks;
and, then, starting two weeks after the last dose of 75 mg, administering, one or more times, to the patient, 150 mg of alirocumab, by subcutaneous injection, every two weeks.


SEQUENCE LISTING (198 sequences)

SEQ ID NO: 1 - protein, 118 aa, synthetic construct [sequence omitted]

SEQ ID NO: 2 - protein, 8 aa, synthetic construct
  REGION 1..8: note = Description of Artificial Sequence: Synthetic peptide
GFTFNNYA

SEQ ID NO: 3 - protein, 8 aa, synthetic construct
  REGION 1..8: note = Description of Artificial Sequence: Synthetic peptide
ISGSGGTT

SEQ ID NO: 4 - protein, 11 aa, synthetic construct
  REGION 1..11: note = Description of Artificial Sequence: Synthetic peptide
AKDSNWGNFDL

SEQ ID NO: 5 - protein, 447 aa, synthetic construct
  REGION 1..447: note = Description of Artificial Sequence: Synthetic REGN727 heavy chain polypeptide
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 113 aa, synthetic construct [sequence omitted]

SEQ ID NO: 7 - protein, 12 aa, synthetic construct
  REGION 1..12: note = Description of Artificial Sequence: Synthetic peptide
QSVLYRSNNRNF

SEQ ID NO: 8 - skipped

SEQ ID NO: 9 - protein, 220 aa, synthetic construct
  REGION 1..220: note = Description of Artificial Sequence: Synthetic REGN727 light chain polypeptide
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 9 aa, synthetic construct
  REGION 1..9: note = Description of Artificial Sequence: Synthetic peptide
QQYYTTPYT

SEQ ID NO: 11 - protein, 127 aa, synthetic construct
  REGION 1..127: note = Description of Artificial Sequence: Synthetic polypeptide
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 12 - protein, 8 aa, synthetic construct
  REGION 1..8: note = Description of Artificial Sequence: Synthetic peptide
GFTFSSHW

SEQ ID NO: 13 - protein, 8 aa, synthetic construct
  REGION 1..8: note = Description of Artificial Sequence: Synthetic peptide
INQDGSEK

SEQ ID NO: 14 - protein, 20 aa, synthetic construct
  REGION 1..20: note = Description of Artificial Sequence: Synthetic peptide
ARDIVLMVYDMDYYYYGMDV

SEQ ID NO: 15 - protein, 112 aa, synthetic construct
  REGION 1..112: note = Description of Artificial Sequence: Synthetic polypeptide
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 16 - protein, 11 aa, synthetic construct
  REGION 1..11: note = Description of Artificial Sequence: Synthetic peptide
QSLLHSNGNNY

SEQ ID NO: 17 - skipped

SEQ ID NO: 18 - protein, 9 aa, synthetic construct
  REGION 1..9: note = Description of Artificial Sequence: Synthetic peptide
MQTLQTPLT

SEQ ID NO: 19 - protein, 8 aa, synthetic construct
  REGION 1..8: note = Description of Artificial Sequence: Synthetic peptide
GFTFSSHW

SEQ ID NO: 20 - protein, 8 aa, synthetic construct
  REGION 1..8: note = Description of Artificial Sequence: Synthetic peptide
INQDGSEK

SEQ ID NO: 21 - protein, 20 aa, synthetic construct
  REGION 1..20: note = Description of Artificial Sequence: Synthetic peptide
ARDIVLMVYDMDYYYYGMDV

SEQ ID NO: 22 - protein, 11 aa, synthetic construct
  REGION 1..11: note = Description of Artificial Sequence: Synthetic peptide
QSLHHSNGNNY

SEQ ID NO: 23 - skipped

SEQ ID NO: 24 - protein, 9 aa, synthetic construct
  REGION 1..9: note = Description of Artificial Sequence: Synthetic peptide
MQTLQTPLT

SEQ ID NO: 25 - protein, 8 aa, synthetic construct
  REGION 1..8: note = Description of Artificial Sequence: Synthetic peptide
GFTFSSHW

SEQ ID NO: 26 - protein, 8 aa, synthetic construct
  REGION 1..8: note = Description of Artificial Sequence: Synthetic peptide
INQDGSEK

SEQ ID NO: 27 - protein, 20 aa, synthetic construct
  REGION 1..20: note = Description of Artificial Sequence: Synthetic peptide
ARDIVLMVYHMDYYYYGMDV

SEQ ID NO: 28 - protein, 11 aa, synthetic construct
  REGION 1..11: note = Description of Artificial Sequence: Synthetic peptide
QSLLHSNGNNY

SEQ ID NO: 29 - skipped

SEQ ID NO: 30 - protein, 9 aa, synthetic construct
  REGION 1..9: note = Description of Artificial Sequence: Synthetic peptide
MQTLQTPLT

SEQ ID NO: 31 - protein, 8 aa, synthetic construct
  REGION 1..8: note = Description of Artificial Sequence: Synthetic peptide
GFTFSSHW

SEQ ID NO: 32 - protein, 8 aa, synthetic construct
  REGION 1..8: note = Description of Artificial Sequence: Synthetic peptide
INQDGSEK

SEQ ID NO: 33 - protein, 20 aa, synthetic construct
  REGION 1..20: note = Description of Artificial Sequence: Synthetic peptide
ARDIVLMVYHMDYYYYGMDV

SEQ ID NO: 34 - protein, 11 aa, synthetic construct
  REGION 1..11: note = Description of Artificial Sequence: Synthetic peptide
QSLHHSNGNNY

SEQ ID NO: 35 - skipped

SEQ ID NO: 36 - protein, 9 aa, synthetic construct
  REGION 1..9: note = Description of Artificial Sequence: Synthetic peptide
MQTLQTPLT

SEQ ID NO: 37 - protein, 131 aa, synthetic construct
  REGION 1..131: note = Description of Artificial Sequence: Synthetic VH; m2CX1D05 polypeptide
QVQLVQSGAEVKKPGSSVKVSCKASGGTFNSHAISWVRQAPGQGLEWMGGINPILGIANYAQKFQGRVTITADESTSTAYMELSSLRSEDTAVYYCARHYEIQIGRYGMNVYYLMYRFASWGQGTLVTVSS

SEQ ID NO: 38 - protein, 10 aa, synthetic construct
  REGION 1..10: note = Description of Artificial Sequence: Synthetic VH CDR1; m2CX1D05 peptide
GGTFNSHAIS

SEQ ID NO: 39 - protein, 20 aa, synthetic construct
  REGION 1..20: note = Description of Artificial Sequence: Synthetic VH CDR2; m2CX1D05 peptide
WMGGINPILGIANYAQKFQG

SEQ ID NO: 40 - protein, 22 aa, synthetic construct
  REGION 1..22: note = Description of Artificial Sequence: Synthetic VH CDR3; m2CX1D05 peptide
HYEIQIGRYGMNVYYLMYRFAS

SEQ ID NO: 41 - protein, 213 aa, synthetic construct
  REGION 1..213: note = Description of Artificial Sequence: Synthetic LC; m2CX1D05 polypeptide
DIQMTQSPSSLSASVGDRVTITCRASQGIRSALNWYQQKPGKAPKLLIYNGSTLQSGVPSRFSGSGSGTDFTLTISSLQPEDFAVYYCQQFDGDPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 42 - protein, 11 aa, synthetic construct
  REGION 1..11: note = Description of Artificial Sequence: Synthetic VL CDR 1; m2CX1D05 peptide
RASQGIRSALN

SEQ ID NO: 43 - protein, 11 aa, synthetic construct
  REGION 1..11: note = Description of Artificial Sequence: Synthetic VL CDR2; m2CX1D05 peptide
LLIYNGSTLQS

SEQ ID NO: 44 - protein, 7 aa, synthetic construct
  REGION 1..7: note = Description of Artificial Sequence: Synthetic VL CDR3; m2CX1D05 peptide
QQFDGDP

SEQ ID NO: 45 - protein, 119 aa, synthetic construct
  REGION 1..119: note = Description of Artificial Sequence: Synthetic VH; 1B20 polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYWYKPLFDIWGQGTLVTVSS

SEQ ID NO: 46 - protein, 10 aa, synthetic construct
  REGION 1..10: note = Description of Artificial Sequence: Synthetic VH CDR1; 1B20 peptide
GYSFTNYWIS

SEQ ID NO: 47 - protein, 20 aa, synthetic construct
  REGION 1..20: note = Description of Artificial Sequence: Synthetic VH CDR2; 1B20 peptide
WMGIIYPGDSYTNYSPSFQG

SEQ ID NO: 48 - protein, 10 aa, synthetic construct
  REGION 1..10: note = Description of Artificial Sequence: Synthetic VH CDR3; 1B20 peptide
DYWYKPLFDI

SEQ ID NO: 49 - protein, 220 aa, synthetic construct
  REGION 1..220: note = Description of Artificial Sequence: Synthetic LC; 1B20 polypeptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYSSFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 50 - protein, 17 aa, synthetic construct
  REGION 1..17: note = Description of Artificial Sequence: Synthetic VL CDR1; 1B20 peptide
RSSQSVLYSSNNKNYLA

SEQ ID NO: 51 - protein, 11 aa, synthetic construct
  REGION 1..11: note = Description of Artificial Sequence: Synthetic VL CDR2; 1B20 peptide
LLIYWASTRES

SEQ ID NO: 52 - protein, 8 aa, synthetic construct
  REGION 1..8: note = Description of Artificial Sequence: Synthetic VL CDR3; 1B20 peptide
QQYSSFPI

SEQ ID NO: 53 - protein, 120 aa, synthetic construct
  REGION 1..120: note = Description of Artificial Sequence: Synthetic variable heavy antibody region polypeptide
EVQLLESGGGLVQPGGSLRLSCKASGYTFSSYGMYWVRQAPGKGLEWIGWIDPGSGGTKYNEKFKGKATISRDNSKNTLYLQMNSLRAEDTAVYYCARERYGYYFDYWGQGTLVTVSSAS

SEQ ID NO: 54 - protein, 16 aa, synthetic construct
  REGION 1..16: note = Description of Artificial Sequence: Synthetic AX132 heavy chain CDR1 antibody region peptide
KASGYTFSSYGMYWVR

SEQ ID NO: 55 - protein, 23 aa, synthetic construct
  REGION 1..23: note = Description of Artificial Sequence: Synthetic AX132 heavy chain CDR2 antibody region peptide
WIGWIDPGSGGTKYNEKFKGKAT

SEQ ID NO: 56 - protein, 15 aa, synthetic construct
  REGION 1..15: note = Description of Artificial Sequence: Synthetic AX132 heavy chain CDR3 antibody region peptide
CARERYGYYFDYWGQ

SEQ ID NO: 57 - protein, 108 aa, synthetic construct
  REGION 1..108: note = Description of Artificial Sequence: Synthetic variable light antibody region polypeptide
EIVLTQSPATLSLSPGERATITCRASQYVGSYLNWYQQKPGQAPRLLIYDASNRATGIPARFSGSGSGTDFTLTISSLEPEDFAVYYCQVWDSSPPVVFGGGTKVEIK

SEQ ID NO: 58 - protein, 17 aa, synthetic construct
  REGION 1..17: note = Description of Artificial Sequence: Synthetic AX213 and AX132 light chain CDR1 antibody region peptide
ITCRASQYVGSYLNWYQ

SEQ ID NO: 59 - protein, 13 aa, synthetic construct
  REGION 1..13: note = Description of Artificial Sequence: Synthetic AX213 and AX132 light chain CDR2 antibody region peptide
LIYDASNRATGIP

SEQ ID NO: 60 - protein, 16 aa, synthetic construct
  REGION 1..16: note = Description of Artificial Sequence: Synthetic AX132 & AX213 light chain CDR3 antibody region peptide
YYCQVWDSSPPVVFGG

SEQ ID NO: 61 - protein, 120 aa, synthetic construct
  REGION 1..120: note = Description of Artificial Sequence: Synthetic variable heavy antibody region polypeptide
EVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSSAS

SEQ ID NO: 62 - protein, 16 aa, synthetic construct
  REGION 1..16: note = Description of Artificial Sequence: Synthetic AX213 heavy chain CDR1 antibody region peptide
KASGYTFSRYGINWVR

SEQ ID NO: 63 - protein, 23 aa, synthetic construct
  REGION 1..23: note = Description of Artificial Sequence: Synthetic AX213 heavy chain CDR2 antibody region peptide
WIGRIDPGNGGTRYNEKFKGKAT

SEQ ID NO: 64 - protein, 15 aa, synthetic construct
  REGION 1..15: note = Description of Artificial Sequence: Synthetic AX213 heavy chain CDR3 antibody region peptide
CARANDGYSFDYWGQ

SEQ ID NO: 65 - protein, 108 aa, synthetic construct; identical to SEQ ID NO: 57
  REGION 1..108: note = Description of Artificial Sequence: Synthetic variable light antibody region polypeptide

SEQ ID NO: 66 - protein, 17 aa, synthetic construct
  REGION 1..17: note = Description of Artificial Sequence: Synthetic AX213 and AX132 light chain CDR1 antibody region peptide
ITCRASQYVGSYLNWYQ

SEQ ID NO: 67 - protein, 13 aa, synthetic construct
  REGION 1..13: note = Description of Artificial Sequence: Synthetic AX213 and AX132 light chain CDR2 antibody region peptide
LIYDASNRATGIP

SEQ ID NO: 68 - protein, 16 aa, synthetic construct
  REGION 1..16: note = Description of Artificial Sequence: Synthetic AX132 & AX213 light chain CDR3 antibody region peptide
YYCQVWDSSPPVVFGG

SEQ ID NO: 69 - protein, 119 aa, synthetic construct
  REGION 1..119: note = Description of Artificial Sequence: Synthetic AX1 VH antibody sequence polypeptide
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSS

SEQ ID NO: 70 - protein, 16 aa, synthetic construct
  REGION 1..16: note = Description of Artificial Sequence: Synthetic AX1 VH CDR1 antibody sequence peptide
KASGFTFTSYYMHWVR

SEQ ID NO: 71 - protein, 23 aa, synthetic construct
  REGION 1..23: note = Description of Artificial Sequence: Synthetic AX1 VH CDR2 antibody sequence peptide
WIGRINPDSGSTKYNEKFKGRAT

SEQ ID NO: 72 - protein, 16 aa, synthetic construct
  REGION 1..16: note = Description of Artificial Sequence: Synthetic AX1 VH CDR3 antibody sequence peptide
CARGGRLSWDFDVWGQ

SEQ ID NO: 73 - protein, 109 aa, synthetic construct
  REGION 1..109: note = Description of Artificial Sequence: Synthetic AX1 VL antibody sequence polypeptide
DIQMTQSPSSLSASVGDRVTITCRASQDISRYLAWYQQKPGKAPKLLIYAASSLQSGVPSRFSGSGSGTDFTLTISSLQPEDFATYYCAAYDYSLGGYVFGDGTKVEIK

SEQ ID NO: 74 - protein, 11 aa, synthetic construct
  REGION 1..11: note = Description of Artificial Sequence: Synthetic AX1 VL CDR1 antibody sequence peptide
RASQDISRYLA

SEQ ID NO: 75 - protein, 7 aa, synthetic construct
  REGION 1..7: note = Description of Artificial Sequence: Synthetic AX1 AX9 AX189 VL CDR2 antibody sequence peptide
AASSLQS

SEQ ID NO: 76 - protein, 11 aa, synthetic construct
  REGION 1..11: note = Description of Artificial Sequence: Synthetic AX1 VL CDR3 antibody sequence peptide
AAYDYSLGGYV

SEQ ID NO: 77 - protein, 121 aa, synthetic construct
  REGION 1..121: note = Description of Artificial Sequence: Synthetic AX9 AX189 VH antibody sequence polypeptide
EVQLLESGGGLVQPGGSLRLSCKASGYTFSSYWMHWVRQAPGKGLEWIGRIDPYNGGTKYNEKFKGKATISRDNSKNTLYLQMNSLRAEDTAVYYCARYGYYLGSYAMDYWGQGTLVTVSS

SEQ ID NO: 78 - protein, 16 aa, synthetic construct
  REGION 1..16: note = Description of Artificial Sequence: Synthetic AX9 AX189 VH CDR1 antibody sequence peptide
KASGYTFSSYWMHWVR

SEQ ID NO: 79 - protein, 23 aa, synthetic construct
  REGION 1..23: note = Description of Artificial Sequence: Synthetic AX9 AX189 VH CDR2 antibody sequence peptide
WIGRIDPYNGGTKYNEKFKGKAT

SEQ ID NO: 80 - protein, 18 aa, synthetic construct
  REGION 1..18: note = Description of Artificial Sequence: Synthetic AX9 AX189 VH CDR3 antibody sequence peptide
CARYGYYLGSYAMDYWGQ

SEQ ID NO: 81 - protein, 109 aa, synthetic construct
  REGION 1..109: note = Description of Artificial Sequence: Synthetic AX189 VL antibody sequence polypeptide
DIQMTQSPSSLSASVGDRVTITCRASQDVSRYLTWYQQKPGKAPKLLIYAASSLQSGVPSRFSGSGSGTDFTLTISSLQPEDFATYYCQAYDYSLSGYVFGGGTKVEIK

SEQ ID NO: 82 - protein, 11 aa, synthetic construct
  REGION 1..11: note = Description of Artificial Sequence: Synthetic AX189 VL CDR1 antibody sequence peptide
RASQDVSRYLT

SEQ ID NO: 83 - protein, 7 aa, synthetic construct
  REGION 1..7: note = Description of Artificial Sequence: Synthetic AX1 AX9 AX189 VL CDR2 antibody sequence peptide
AASSLQS

SEQ ID NO: 84 - protein, 11 aa, synthetic construct
  REGION 1..11: note = Description of Artificial Sequence: Synthetic AX189 VL CDR3 antibody sequence peptide
QAYDYSLSGYV

SEQ ID NO: 85 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 86 - protein, 5 aa, Homo sapiens
SYGIS

SEQ ID NO: 87 - protein, 17 aa, Homo sapiens
WVSFYNGNTNYAQKLQG

SEQ ID NO: 88 - protein, 6 aa, Homo sapiens
GYGMDV

SEQ ID NO: 89 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 90 - protein, 14 aa, Homo sapiens
TGTSSDVGGYNSVS

SEQ ID NO: 91 - protein, 7 aa, Homo sapiens
EVSNRPS

SEQ ID NO: 92 - protein, 9 aa, Homo sapiens
NSYTSTSMV

SEQ ID NO: 93 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 94 - protein, 10 aa, Homo sapiens
GFTFSSYSMN

SEQ ID NO: 95 - protein, 17 aa, Homo sapiens
SISSSSSYISYADSVKG

SEQ ID NO: 96 - protein, 14 aa, Homo sapiens
DYDFWSAYYDAFDV

SEQ ID NO: 97 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 98 - protein, 14 aa, Homo sapiens
TGSSSNIGAGYDVH

SEQ ID NO: 99 - protein, 7 aa, Homo sapiens
GNSNRPS

SEQ ID NO: 100 - protein, 11 aa, Homo sapiens
QSYDSSLSGSV

SEQ ID NO: 101 - protein, 114 aa, Homo sapiens
QVQLVESGGGVAQPGRSLRLSCAASGFTFSSYGMHWVRQAPGKGLEWVAVIYYDGINKHYADSVKGRFTISRDNSKNTLYLQMNSLRAEDTAVYYCARDRGLDWGQGTLVTVSS

SEQ ID NO: 102 - protein, 10 aa, Homo sapiens
GFTFSSYGMH

SEQ ID NO: 103 - protein, 17 aa, Homo sapiens
VIYYDGINKHYADSVKG

SEQ ID NO: 104 - protein, 5 aa, Homo sapiens
DRGLD

SEQ ID NO: 105 - protein, 113 aa, Homo sapiens
DIVMTQSPDSLAVSLGERATINCKSSQSVLYSSNSKNYLVWYQQKPGQPPKLLIYWASTRESGVPDRFSGSGSGTDFTLTISSLQAEDVAVYYCQQYYSTPWTFGQGTKVEIK

SEQ ID NO: 106 - protein, 17 aa, Homo sapiens
KSSQSVLYSSNSKNYLV

SEQ ID NO: 107 - protein, 7 aa, Homo sapiens
WASTRES

SEQ ID NO: 108 - protein, 9 aa, Homo sapiens
QQYYSTPWT

SEQ ID NO: 109 - protein, 118 aa, synthetic construct
  REGION 1..118: note = Description of Artificial Sequence: Synthetic anti-PCSK9 monoclonal antibody pJG04 (clones LGT-209 and LGT-210) Vh heavy chain variable region (FR1-FR4) polypeptide
QVQLVQSGAEVKKPGASVKVSCKASGYTFSTMYMSWVRQAPGQGLEWMGRIDPANEHTNYAQKFQGRVTMTRDTSISTAYMELSRLTSDDTAVYYCARSYYYYNMDYWGQGTLVTVSS

SEQ ID NO: 110 - protein, 5 aa, synthetic construct
  REGION 1..5: note = Description of Artificial Sequence: Synthetic anti-PCSK9 monoclonal antibody clones LGT-209, LGT-210 and LGT-211 heavy chain CDR1 peptide
TMYMS

SEQ ID NO: 111 - protein, 17 aa, synthetic construct
  REGION 1..17: note = Description of Artificial Sequence: Synthetic anti-PCSK9 monoclonal antibody clones LGT-209, LGT-210 and LGT-211 heavy chain CDR2 peptide
RIDPANEHTNYAQKFQG

SEQ ID NO: 112 - protein, 9 aa, synthetic construct
  REGION 1..9: note = Description of Artificial Sequence: Synthetic anti-PCSK9 monoclonal antibody pJG04(clones LGT-209 and LGT-210) Vh heavy chain complementarity determining region 3 (CDR3) peptide
SYYYYNMDY

SEQ ID NO: 113 - protein, 106 aa, synthetic construct
  REGION 1..106: note = Description of Artificial Sequence: Synthetic anti-PCSK9 monoclonal antibody pJG10(clones LGT-209 and LGT-211) Vk light chain variable region (FR1-FR4) polypeptide
QIVLTQSPATLSVSPGERATLSCRASQSVSYMHWYQQKPGQAPRLLIYGVFRRATGIPDRFSGSGSGTDFTLTIGRLEPEDFAVYYCLQWSSDPPTFGQGTKLEIK

SEQ ID NO: 114 - protein, 10 aa, synthetic construct
  REGION 1..10: note = Description of Artificial Sequence: Synthetic anti-PCSK9 monoclonal antibody clones LGT-209, LGT-210 and LGT-211 light chain CDR1 peptide
RASQSVSYMH

SEQ ID NO: 115 - protein, 7 aa, synthetic construct
  REGION 1..7: note = Description of Artificial Sequence: Synthetic anti-PCSK9 monoclonal antibody clones LGT-209, LGT-210 and LGT-211 light chain CDR1 peptide
GVFRRAT

SEQ ID NO: 116 - protein, 9 aa, synthetic construct
  REGION 1..9: note = Description of Artificial Sequence: Synthetic mouse anti-PCSK9 monoclonal antibody LFU720 and anti-PCSK9 monoclonal antibody clones LGT-209, LGT-210 and LGT-211 light chain CDR3 peptide
LQWSSDPPT

SEQ ID NO: 117 - protein, 118 aa, Homo sapiens
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 118 - protein, 7 aa, synthetic construct
  REGION 1..7: note = Description of Artificial Sequence: Synthetic peptide
GYTFTSY

SEQ ID NO: 119 - protein, 6 aa, synthetic construct
  REGION 1..6: note = Description of Artificial Sequence: Synthetic peptide
SPFGGR

SEQ ID NO: 120 - protein, 9 aa, synthetic construct
  REGION 1..9: note = Description of Artificial Sequence: Synthetic variable heavy chain CDR peptide
ERPLYASDL

SEQ ID NO: 121 - protein, 107 aa, Homo sapiens
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 122 - protein, 11 aa, synthetic construct
  REGION 1..11: note = Description of Artificial Sequence: Synthetic variable light chain CDR peptide
RASQGISSALA

SEQ ID NO: 123 - protein, 7 aa, synthetic construct
  REGION 1..7: note = Description of Artificial Sequence: Synthetic variable light chain CDR peptide
SASYRYT

SEQ ID NO: 124 - protein, 9 aa, synthetic construct
  REGION 1..9: note = Description of Artificial Sequence: Synthetic variable light chain CDR peptide
QQRYSLWRT

SEQ ID NO: 125 - protein, 118 aa, Homo sapiens
QVQLVQSGAEVKKPGASVKVSCKASGYTFTSYYMHWVRQAPGQGLEWMGEIHPSGGRTNYNEKFKSRVTMTRDTSTSTVYMELSSLRSEDTAVYYCARERPLYAMDYWGQGTTVTVSS

SEQ ID NO: 126 - protein, 10 aa, Homo sapiens
GYTFTSYYMH

SEQ ID NO: 127 - protein, 17 aa, Homo sapiens
EIHPSGGRTNYNEKFKS

SEQ ID NO: 128 - protein, 9 aa, Homo sapiens
ERPLYAMDY

SEQ ID NO: 129 - protein, 107 aa, Homo sapiens [sequence omitted]

SEQ ID NO: 130 - protein, 11 aa, Homo sapiens
KASQDVHTAVA

SEQ ID NO: 131 - protein, 7 aa, Homo sapiens
HASYRYT

SEQ ID NO: 132 - protein, 9 aa, Homo sapiens
QQRYSLWRT

SEQ ID NO: 133 - protein, 118 aa, Homo sapiens [sequence omitted]

SEQ ID NO: 134 - protein, 10 aa, Homo sapiens
GYTFTSYYMH

SEQ ID NO: 135 - protein, 17 aa, Homo sapiens
EIHPSGGRTNYNEKFKS

SEQ ID NO: 136 - protein, 9 aa, Homo sapiens
ERPLYASDL

SEQ ID NO: 137 - protein, 107 aa, Homo sapiens [sequence omitted]

SEQ ID NO: 138 - protein, 11 aa, Homo sapiens
KASQDVHTAVA

SEQ ID NO: 139 - protein, 7 aa, Homo sapiens
HASYRYT

SEQ ID NO: 140 - protein, 9 aa, Homo sapiens
QQRYSLWRT

SEQ ID NO: 141 - protein, 118 aa, Mus musculus
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCARERPLYAMDYWGQGTSVTVSS

SEQ ID NO: 142 - protein, 7 aa, Mus musculus
GYTFTSY

SEQ ID NO: 143 - protein, 6 aa, Mus musculus
NPSNGR

SEQ ID NO: 144 - protein, 9 aa, Mus musculus
ERPLYAMDY

SEQ ID NO: 145 - protein, 108 aa, Mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQRYSTPRTFGGGTKLEIKR

SEQ ID NO: 146 - protein, 11 aa, Mus musculus
KASQDVSTAVA

SEQ ID NO: 147 - protein, 7 aa, synthetic construct
  REGION 1..7: note = Description of Artificial Sequence: Synthetic variable light chain CDR peptide
SASYRYT

SEQ ID NO: 148 - protein, 9 aa, Mus musculus
QQRYSTPRT

SEQ ID NO: 149 - protein, 115 aa, Mus musculus
EVQLQQSGPELVKPGASVKISCKASGYTFTDYYMNWVKQSHGKSLEWIGDINPNNGGTTYNQKFKGKATLTVDKSYSTAYMELRSLTSEDSAVYYCARWLLFAYWGQGTLVTVSA

SEQ ID NO: 150 - protein, 7 aa, Mus musculus
GYTFTDY

SEQ ID NO: 151 - protein, 6 aa, Mus musculus
NPNNGG

SEQ ID NO: 152 - protein, 6 aa, Mus musculus
WLLFAY

SEQ ID NO: 153 - protein, 108 aa, Mus musculus
DIVMTQSQKFMSTSVGDRVSVTCKASQNVGTNVAWYQQKPGQSPKALIYSASYRYSGVPDRFTGSGSGTDFTLTISNVLSEDLAEYFCQQFYSYPYTFGGGTKLEIKR

SEQ ID NO: 154 - protein, 11 aa, Mus musculus
KASQNVGTNVA

SEQ ID NO: 155 - protein, 7 aa, Mus musculus
SASYRYS

SEQ ID NO: 156 - protein, 9 aa, Mus musculus
QQFYSYPYT

SEQ ID NO: 157 - protein, 123 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAGGGIYYRYDRNYFDYWGQGTTLTVSS

SEQ ID NO: 158 - protein, 7 aa, Mus musculus
GYTFTDY

SEQ ID NO: 159 - protein, 6 aa, Mus musculus
NPNNGG

SEQ ID NO: 160 - protein, 14 aa, Mus musculus
GGIYYRYDRNYFDY

SEQ ID NO: 161 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 162 - protein, 11 aa, Mus musculus
SASQGISNYLN

SEQ ID NO: 163 - protein, 7 aa, Mus musculus
YTSSLHS

SEQ ID NO: 164 - protein, 9 aa, synthetic construct
  REGION 1..9: note = Description of Artificial Sequence: Synthetic peptide
QQYSKLPFT

SEQ ID NO: 165 - protein, 117 aa, Mus musculus
EVKLVESEGGLVQPGSSMKLSCTASGFTFSDYYMAWVRQVPEKGLEWVANINYDGSNTSYLDSLKSRFIISRDNAKNILYLQMSSLKSEDTATYYCAREKFAAMDYWGQGTSVTVSS

SEQ ID NO: 166 - protein, 7 aa, Mus musculus
GFTFSDY

SEQ ID NO: 167 - protein, 6 aa, Mus musculus
NYDGSN

SEQ ID NO: 168 - protein, 8 aa, Mus musculus
EKFAAMDY

SEQ ID NO: 169 - protein, 108 aa, Mus musculus
DIVMTQSHKFMSTSFGDRVSITCKASQDVSNALAWYQQKPGHSPKLLIFSASYRYTGVPDRFTGSGSGTDFTFTISSVQAEDLAVYYCQQHYSTPWTFGGGTKLEIKR

SEQ ID NO: 170 - protein, 11 aa, Mus musculus
KASQDVSNALA

SEQ ID NO: 171 - protein, 7 aa, synthetic construct
  REGION 1..7: note = Description of Artificial Sequence: Synthetic variable light chain CDR peptide
SASYRYT

SEQ ID NO: 172 - protein, 9 aa, Mus musculus
QQHYSTPWT

SEQ ID NO: 173 - protein, 121 aa, synthetic construct
  REGION 1..121: note = Description of Artificial Sequence: Synthetic polypeptide
EVQLVESGGGLVQPGGSLRLSCAASGFTFTRHTIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 174 - protein, 10 aa, synthetic construct
  REGION 1..10: note = Description of Artificial Sequence: Synthetic peptide
GFTFTRHTIH

SEQ ID NO: 175 - protein, 17 aa, synthetic construct
  REGION 1..17: note = Description of Artificial Sequence: Synthetic peptide
RISPANGNTNYADSVKG

SEQ ID NO: 176 - protein, 12 aa, synthetic construct
  REGION 1..12: note = Description of Artificial Sequence: Synthetic peptide
WIGSRELYIMDY

SEQ ID NO: 177 - protein, 108 aa, synthetic construct
  REGION 1..108: note = Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYRIQPTFGQGTKVEIKR

SEQ ID NO: 178 - protein, 11 aa, synthetic construct
  REGION 1..11: note = Description of Artificial Sequence: Synthetic peptide
RASQDVSTAVA

SEQ ID NO: 179 - protein, 7 aa, synthetic construct
  REGION 1..7: note = Description of Artificial Sequence: Synthetic peptide
SASFLYS

SEQ ID NO: 180 - protein, 9 aa, synthetic construct
  REGION 1..9: note = Description of Artificial Sequence: Synthetic peptide
QQSYRIQPT

SEQ ID NO: 181 - protein, 121 aa, synthetic construct
  REGION 1..121: note = Description of Artificial Sequence: Synthetic polypeptide
EVQLVESGGGLVQPGGSLRLSCAASGFTFSSTAIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 182 - protein, 10 aa, synthetic construct
  REGION 1..10: note = Description of Artificial Sequence: Synthetic peptide
GFTFSSTAIH

SEQ ID NO: 183 - protein, 17 aa, synthetic construct
  REGION 1..17: note = Description of Artificial Sequence: Synthetic peptide
RISPANGNTNYADSVKG

SEQ ID NO: 184 - protein, 12 aa, synthetic construct
  REGION 1..12: note = Description of Artificial Sequence: Synthetic peptide
WIGSRELYIMDY

SEQ ID NO: 185 - protein, 108 aa, synthetic construct
  REGION 1..108: note = Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPALHTFGQGTKVEIKR

SEQ ID NO: 186 - protein, 11 aa, synthetic construct
  REGION 1..11: note = Description of Artificial Sequence: Synthetic peptide
RASQDVSTAVA

SEQ ID NO: 187 - protein, 7 aa, synthetic construct
  REGION 1..7: note = Description of Artificial Sequence: Synthetic peptide
SASFLYS

SEQ ID NO: 188 - protein, 9 aa, synthetic construct
  REGION 1..9: note = Description of Artificial Sequence: Synthetic peptide
QQSYPALHT

SEQ ID NO: 189 - protein, 125 aa, synthetic construct
  REGION 1..125: note = Description of Artificial Sequence: Synthetic polypeptide
EVQLVESGGGLVKPGGSLRLSCAASGFPFSKLGMVWVRQAPGKGLEWVSTISSGGGYTYYPDSVKGRFTISRDNAKNSLYLQMNSLRAEDTAVYYCAREGISFQGGTYTYVMDYWGQGTLVTVSS

SEQ ID NO: 190 - protein, 10 aa, synthetic construct
  REGION 1..10: note = Description of Artificial Sequence: Synthetic peptide
GFPFSKLGMV

SEQ ID NO: 191 - protein, 17 aa, synthetic construct
  REGION 1..17: note = Description of Artificial Sequence: Synthetic peptide
TISSGGGYTYYPDSVKG

SEQ ID NO: 192 - protein, 16 aa, synthetic construct
  REGION 1..16: note = Description of Artificial Sequence: Synthetic peptide
EGISFQGGTYTYVMDY

SEQ ID NO: 193 - protein, 112 aa, synthetic construct
  REGION 1..112: note = Description of Artificial Sequence: Synthetic polypeptide
DIVMTQSPLSLPVTPGEPASISCRSSKSLLHRNGITYSYWYLQKPGQSPQLLIYQLSNLASGVPDRFSGSGSGTDFTLKISRVEAEDVGVYYCYQNLELPLTFGQGTKVEIK

SEQ ID NO: 194 - protein, 16 aa, synthetic construct
  REGION 1..16: note = Description of Artificial Sequence: Synthetic peptide
RSSKSLLHRNGITYSY

SEQ ID NO: 195 - protein, 7 aa, synthetic construct
  REGION 1..7: note = Description of Artificial Sequence: Synthetic peptide
QLSNLAS

SEQ ID NO: 196 - protein, 9 aa, synthetic construct
  REGION 1..9: note = Description of Artificial Sequence: Synthetic peptide
YQNLELPLT

SEQ ID NO: 197 - DNA, 2076 bp, Homo sapiens
  source 1..2076: mol_type = genomic DNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccag

SEQ ID NO: 198 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ